SEQUENCE LISTING (19 sequences)

SEQ ID NO: 1 - protein, 142 aa, Homo sapiens
MGAPTLPPAWQPFLKDHRISTFKNWPFLEGCACTPERMAEAGFIHCPTENEPDLAQCFFCFKELEGWEPDDDPIEEHKKHSSGCAFLSVKKQFEELTLGEFLKLDRERAKNKIAKETNNKKKEFEETAKKVRRAIEQLAAMD

SEQ ID NO: 2 - protein, 9 aa, Homo sapiens
FEELTLGEF

SEQ ID NO: 3 - protein, 9 aa, Artificial Sequence
  Description of Artificial Sequence Synthetic peptide
FTELTLGEF

SEQ ID NO: 4 - protein, 9 aa, Homo sapiens
LTLGEFLKL

SEQ ID NO: 5 - protein, 9 aa, Artificial Sequence
  Description of Artificial Sequence Synthetic peptide
LMLGEFLKL

SEQ ID NO: 6 - protein, 10 aa, Homo sapiens
RISTFKNWPF

SEQ ID NO: 7 - protein, 10 aa, Artificial Sequence
  Description of Artificial Sequence Synthetic peptide
RISTFKNWPK

SEQ ID NO: 8 - protein, 9 aa, Artificial Sequence
  Description of Artificial Sequence Synthetic peptide
STFKNWPFL

SEQ ID NO: 9 - protein, 9 aa, Artificial Sequence
  Description of Artificial Sequence Synthetic peptide
LPPAWQPFL

SEQ ID NO: 10 - protein, 16 aa, Artificial Sequence
  Description of Artificial Sequence Synthetic peptide
AQYIKANSKFIGITEL

SEQ ID NO: 11 - DNA, 429 bp, Homo sapiens
atgggtgccccgacgttgccccctgcctggcagccctttctcaaggaccaccgcatctctacattcaagaactggcccttcttggagggctgcgcctgcaccccggagcggatggccgaggctggcttcatccactgccccactgagaacgagccagacttggcccagtgtttcttctgcttcaaggagctggaaggctgggagccagatgacgaccccatagaggaacataaaaagcattcgtccggttgcgctttcctttctgtcaagaagcagtttgaagaattaacccttggtgaatttttgaaactggacagagaaagagccaagaacaaaattgcaaaggaaaccaacaataagaagaaagaatttgaggaaactgcgaagaaagtgcgccgtgccatcgagcagctggctgccatggattga

SEQ ID NO: 12 - protein, 13 aa, Artificial Sequence
  Description of Artificial Sequence Synthetic peptide
  MOD_RES (3)..(3): Cyclohexylalanyl
AKXVAAWTLKAAA

SEQ ID NO: 13 - protein, 21 aa, Clostridium tetani
FNNFTVSFWLRVPKVSASHLE

SEQ ID NO: 14 - DNA, 20 bp, Artificial Sequence
  Description of Artificial Sequence Synthetic oligonucleotide
tccatgacgttcctgacgtt

SEQ ID NO: 15 - protein, 4 aa, Artificial Sequence
  Description of Artificial Sequence Synthetic peptide
CGPC

SEQ ID NO: 16 - protein, 5 aa, Artificial Sequence
  Description of Artificial Sequence Synthetic peptide
  MOD_RES (1)..(1): Pam3Cys
XSKKK

SEQ ID NO: 17 - DNA, 26 bp, Artificial Sequence
  Description of Artificial Sequence Synthetic oligonucleotide
  modified_base (1)..(1): Inosine
  modified_base (3)..(3): Inosine
  modified_base (5)..(5): Inosine
  modified_base (7)..(7): Inosine
  modified_base (9)..(9): Inosine
  modified_base (11)..(11): Inosine
  modified_base (13)..(13): Inosine
  modified_base (15)..(15): Inosine
  modified_base (17)..(17): Inosine
  modified_base (19)..(19): Inosine
  modified_base (21)..(21): Inosine
  modified_base (23)..(23): Inosine
  modified_base (25)..(25): Inosine
ncncncncncncncncncncncncnc

SEQ ID NO: 18 - protein, 6 aa, Artificial Sequence
  Description of Artificial Sequence Synthetic peptide
  MOD_RES (1)..(1): Pam2Cys
XSKKKK

SEQ ID NO: 19 - protein, 6 aa, Artificial Sequence
  Description of Artificial Sequence Synthetic peptide
  MOD_RES (1)..(1): Pam3Cys
XSKKKK

(19) United States
(12) Patent Application Publication
(10) Pub. No.: US 2023/0381309 A1
(43) Pub. Date: Nov. 30, 2023

(54) METHODS OF TREATING DIFFUSE LARGE B-CELL LYMPHOMA

(71) Applicant: IMMUNOVACCINE TECHNOLOGIES INC., Dartmouth (CA)

(72) Inventors: Stephan FISET, Levis (CA); Yogesh BRAMHECHA, Halifax (CA); Rebekah CONLON, Dartmouth (CA); Neil BERINSTEIN, Toronto (CA)

(21) Appl. No.: 18/031,479

(22) PCT Filed: Oct. 13, 2021
(86) PCT No.: PCT/IB2021/000688

§ 371 Date: Apr. 12, 2023

Related U.S. Application Data
(60) Provisional application No. 63/091,061, filed on Oct. 13, 2020.
(60) Provisional application No. 63/110,743, filed on Nov. 6, 2020.
(60) Provisional application No. 63/121,486, filed on Dec. 4, 2020.

(51) Int. Cl. A61K 39/395; A61P 35/00; G01N 33/68; C07K 16/28; A61K 39/00; A61K 31/675; A61K 47/44

(52) U.S. Cl. CPC A61K 39/3955; A61P 35/00; G01N 33/6872; C07K 16/2818; A61K 39/00115; A61K 31/675; A61K 47/44; C07K 2317/76; A61K 2039/545; A61K 2039/572; A61K 2039/55561; G01N 2333/70521; A61K 2039/505; C07K 2317/24

(57) ABSTRACT

The present application relates generally to methods for treating hematologic malignancy such as diffuse large B cell lymphoma (DLBCL), and in particular to methods involving detecting the expression of Programmed Death-Ligand 1 (PD-L1) in a biological sample of the subject and administering a T cell activation therapeutic with an inhibitor of PD-L1 or Programmed Death 1 (PD-1). It was surprisingly found that the level of PD-L1 expression correlates with the clinical responses with the T cell activation therapeutic together with an inhibitor of PD-L1 or PD-1 in the treatment of hematologic malignancies, thus, making PD-L1 an unexpected biomarker for the treatment of DLBCL.

### CROSS-REFERENCE TO RELATED APPLICATIONS

This application claims priority to U.S. Provisional Application No. 63/091,061, filed Oct. 13, 2020, U.S. Provisional Application No. 63/110,743, filed Nov. 6, 2020, and U.S. Provisional Application No. 63/121,486, filed Dec. 4, 2020, the disclosure of each of which is herein incorporated by reference in its entirety.

### SEQUENCE LISTING

The instant application contains a Sequence Listing which has been submitted electronically in ASCII format and is hereby incorporated by reference in its entirety. Said ASCII copy, created on Oct. 13, 2021, is named 249979_000098_SL.TXT and is 6,990 bytes in size.

### FIELD OF THE INVENTION

The present application relates generally to methods for treating hematologic malignancy such as diffuse large B cell lymphoma (DLBCL).

### BACKGROUND

Treatment for diffuse large B cell lymphoma (DLBCL), especially relapsed/refractory DLBCL, presents an unmet medical need. Most patients with this diagnosis are chemotherapy resistant or refractory and have failed first line chemotherapy and possibly autologous stem cell transplantation¹. Unfortunately, the efficacy of anti-PD-1 of anti-PD-L1 as monotherapy in the treatment of r/r DLBCL has been very limited. Novel treatment approaches that target other pathways that may be driving these resistant lymphomas are important. Although chimeric antigen receptor (CAR)-T cells are a promising new treatment approach, many patients will not be able to receive this therapy either because of the toxicity profile and comorbid illnesses or availability of this complex treatment approach². Accordingly, there exists a need for a predictive biomarker and alternative therapies for treating DLBCL.

### SUMMARY OF THE INVENTION

As specified in the Background section above, there is a great need in the art for a predictive biomarker and alternative therapies for treating DLBCL. The present application addresses these and other needs.

In one aspect, the invention provides a method of treating a hematologic malignancy in a subject in need thereof, said method comprising (a) detecting the expression of biomarker Programmed Death-Ligand 1 (PD-L1) in a biological sample of the subject and (b) administering to the subject a therapeutically effective amount of an inhibitor of PD-L1 or Programmed Death 1 (PD-1), and a therapeutically effective amount of a T cell activation therapeutic, wherein PD-L1 expression is detected in the biological sample. In some embodiments, the method further comprises obtaining a biological sample from the subject prior to the detecting step.

In some embodiments, the biological sample is a tumor sample. In some embodiments, PD-L1 expression is detected in, for example, at least 1% of the cells in the biological sample, in at least 5% of the cells in the biological sample, or in at least 10% of the cells in the biological sample. In some embodiments, the cells are tumor cells, lymphocytes and/or macrophages. In some embodiments, the cells are tumor cells. In some embodiments, the cells are CD20+ cells.

In some embodiments, the detection of PD-L1 expression is performed using a multiplex immunofluorescence (mIF) assay, an immunohistochemistry (IHC) assay, a fluorescence in situ hybridization (FISH) assay, RNAscope, or flow cytometry.

In some embodiments, the inhibitor of PD-1 or PD-L1 is an antibody. In some embodiments, the inhibitor of PD-1 or PD-L1 is pembrolizumab, nivolumab, pidilizumab, AMP-224, RMP1-4, J43, atezolizumab, avelumab, BMS-936559, durvalumab, tislelizumab, cemiplimab, or a combination thereof. In some embodiments, the inhibitor of PD-1 is pembrolizumab.

In some embodiments, the inhibitor of PD-1 or PD-L1 is administered about every 1 to 9 weeks or about every 1 to 6 weeks. In some embodiments, the inhibitor of PD-1 or PD-L1 is administered, for example, every 2 weeks, every 3 weeks, every 4 weeks, or every 6 weeks. In one embodiment, the inhibitor of PD-1 or PD-L1 is administered every 3 weeks. In some embodiments, the inhibitor of PD-1 or PD-L1 is administered at about 50 mg per dose to about 1500 mg per dose. In some embodiments, the inhibitor of PD-1 or PD-L1 is administered at about 100 mg per dose, about 200 mg per dose, about 400 mg per dose, about 480 mg per dose, or about 1200 mg per dose. In some embodiments, the inhibitor of PD-1 or PD-L1 is administered at an amount less than 300 mg per dose. In some embodiments, the inhibitor of PD-1 or PD-L1 is administered at about 200 mg/day.

In some embodiments, a first dose of the inhibitor of PD-1 or PD-L1 is administered to the subject followed by one or more maintenance doses of the inhibitor of PD-1 or PD-L1. In some embodiments, the inhibitor of PD-1 or PD-L1 is administered before, after, or concurrently with the T cell activation therapeutic. In some embodiments, the first dose of the inhibitor of PD-1 or PD-L1 is administered to the subject on the same day as the first dose of the T cell activation therapeutic. In some embodiments, the first dose of the inhibitor of PD-1 or PD-L1 is administered to the subject after the first dose of the T cell activation therapeutic. In some embodiments, the first dose of the inhibitor of PD-1 or PD-L1 is administered to the subject the day after the first dose of the T cell activation therapeutic. In some embodiments, the first dose of the inhibitor of PD-1 or PD-L1 is administered to the subject before the first dose of the T cell activation therapeutic. In some embodiments, the first dose of the inhibitor of PD-1 or PD-L1 is administered to the subject the day before the first dose of the T cell activation therapeutic. In some embodiments, administration of the inhibitor of PD-1 or PD-L1 continues during the course of administering the T cell activation therapeutic.

In some embodiments, the inhibitor of PD-1 or PD-L1 is administered by injection to the subject. In some embodiments, the injection is an intravenous, subcutaneous, intertumoral, or intramuscular injection.

In some embodiments, the T cell activation therapeutic comprises at least one survivin antigen. In some embodiments, the survivin antigen is a peptide antigen or a nucleic acid encoding the peptide antigen. In some embodiments, the survivin antigen is a peptide antigen comprising an amino acid sequence from the survivin protein (SEQ ID NO: 1) that is capable of eliciting a cytotoxic T-lymphocyte (CTL) response in the subject, or a nucleic acid molecule encoding said peptide antigen. In some embodiments, the survivin antigen is a peptide antigen comprising at least one of amino acid sequence FEELTLGEF (SEQ ID NO: 2); FTELTLGEF (SEQ ID NO: 3); LTLGEFLKL (SEQ ID NO: 4); LMLGEFLKL (SEQ ID NO: 5); RISTFKNWPF (SEQ ID NO: 6); RISTFKNWPK (SEQ ID NO: 7); STFKNWPFL (SEQ ID NO: 8); or LPPAWQPFL (SEQ ID NO: 9), or a nucleic acid molecule encoding said peptide antigen. In some embodiments, the at least one survivin antigen comprises a mixture of five peptide antigens comprising the amino acid sequence FTELTLGEF (SEQ ID NO: 3); LMLGEFLKL (SEQ ID NO: 5); RISTFKNWPK (SEQ ID NO: 7); STFKNWPFL (SEQ ID NO: 8) or LPPAWQPFL (SEQ ID NO: 9).

In some embodiments, the at least one survivin antigen is administered at a concentration of about 0.1 mg/ml to about 5 mg/ml for each peptide antigen. In some embodiments, the least one survivin antigen is administered at a concentration of about 1 mg/ml for each peptide antigen. In some embodiments, the T cell activation therapeutic is administered at a dose of about 0.01 ml to about 1 ml or at a dose of about 0.25 ml or about 0.5 ml. In some embodiments, the T cell activation therapeutic antigen is administered a priming dose of about 0.01 ml to about 1 ml or at a priming dose of about 0.25 ml or about 0.5 ml. In some embodiments, the T cell activation therapeutic is administered a booster dose of about 0.01 ml to about 1 ml. In some embodiments, the T cell activation therapeutic is administered at a booster dose of about 0.1 ml.

In some embodiments, the T cell activation therapeutic is a composition comprising the at least one survivin antigen, lipid vesicle particles, and a carrier comprising a continuous phase of a hydrophobic substance.

In some embodiments, the composition further comprises a T-helper epitope. In some embodiments, the T-helper epitope is a peptide comprising the amino acid sequence AQYIKANSKFIGITEL (SEQ ID NO: 10).

In some embodiments, the composition further comprises an adjuvant. In some embodiments, the adjuvant is a polynucleotide, wherein the polynucleotide is DNA or RNA based. In some embodiments, the adjuvant is a polyI.C polynucleotide and wherein the polyI.C polynucleotide is DNA or RNA based.

In some embodiments, the hydrophobic substance is a vegetable oil, nut oil, or mineral oil. In some embodiments, the carrier is mineral oil or is a mannide oleate in a mineral oil solution. In some embodiments, the carrier is Montanide® ISA 51.

In some embodiments, the T cell activation therapeutic is administered by injection to the subject. In some embodiments, injection is a subcutaneous injection.

In some embodiments, wherein step b) further comprises administering an effective amount of one or more active agent to the subject. In some embodiments, the active agent is an agent that interferes with DNA replication. In some embodiments, the active agent is capable of selectively targeting rapidly dividing cells of the immune system and causing programmed cell death. In some embodiments, the active agent is an alkylating agent. In some embodiments, the alkylating agent is a nitrogen mustard alkylating agent. In some embodiments, the nitrogen mustard alkylating agent is cyclophosphamide.

In some embodiments, the active agent is at least one of gemcitabine, 5-FU, cisplatin, oxaliplatin, temozolomide, paclitaxel, capecitabine, methotrexate, epirubicin, idarubicin, mitoxantrone, bleomycin, decitabine, or docetaxel. In some embodiments, the active agent is at least one of thalidomide, bortezomib, IL-2, IL-12, IL-15, IFN-gamma, IFN-alpha, TNF-alpha, metformin, or lenalidomide. In some embodiments, the active agent is an inhibitor of at least one of VEGF, a VEGFR, or CD40. In some embodiments, the active agent improves the efficacy of the T cell activation therapeutic by directly enhancing the immune response against the antigen, such as by increasing the activity or number of antigen-specific CD8+ T cells. In some embodiments, increasing the activity or number of antigen-specific CD8+ T cells involves an enrichment of antigen-specific CD8+ T cells due to a relative decrease in total CD8+ T cells.

In some embodiments, the active agent improves the efficacy of the T cell activation therapeutic by reducing the number or activity of suppressive immune cells, for example CD4+FoxP3+ regulatory T cells (Tregs), myeloid-derived suppressor cells (MDSCs), and/or CD19+CD1d+CD5+ B cells (Bregs).

In some embodiments, the active agent is administered orally to the subject. In some embodiments, the active agent is administered by injection to the subject. In some embodiments, the injection is an intravenous, subcutaneous, intertumoral, or intramuscular injection.

In some embodiments, the effective amount of the active agent is an amount sufficient to provide an immune-modulating effect. In some embodiments, the active agent is administered at about 25-300 mg/day, about 50-100 mg/day, or about 100 mg/day. In some embodiments, the active agent is administered at about 50 mg per dose. In some embodiments, the active agent is administered to the subject at least 1, 2, 3, or 4 times daily. In some embodiments, the active agent is administered twice a day. In some embodiments, the active agent is administered before, after, or concurrently with the T cell activation therapeutic.

In some embodiments, step b) comprises administering a first dose of the active agent to the subject at least two days, three days, four days, five days, or six days prior to administering the T cell activation therapeutic. In some embodiments, step b) comprises administering a first dose of the active agent to the subject about one week prior to administering the T cell activation therapeutic. In some embodiments, step b) comprises administering to the subject a first dose of the active agent, followed by one or more maintenance doses of the active agent. In some embodiments, step b) comprises administering the active agent to the subject twice daily for a period of about one week. In some embodiments, step b) comprises administering the active agent to the subject in a low dose metronomic regimen. In some embodiments, the metronomic regimen comprises administering the active agent to the subject daily for a period of about one week every second week. In certain embodiments, the active agent is administered twice daily. In some embodiments, the metronomic regimen comprises administering the active agent for a two-week cycle, wherein the active agent is administered to the subject during the first week of the cycle, wherein the active agent is not administered to the subject during the second week of the cycle, and wherein the metronomic regimen comprises at least two cycles. In some embodiments, step b) comprises administering the T cell activation therapeutic to the subject about once every three weeks. In some embodiments, step b) comprises administering the T cell activation therapeutic to the subject about once every eight weeks. In some embodiments, step b) comprises administering first two doses of the T cell activation therapeutic to the subject about three weeks apart, and then administering the T cell activation therapeutic to the subject about once every eight weeks. In some embodiments, the method comprises administering the active agent to the subject beginning about one week before administering a first dose of the T cell activation therapeutic, and administering the T cell activation therapeutic to the subject about once every three weeks. In some embodiments, the method comprises administering the active agent to the subject beginning about one week before administering a first dose of the T cell activation therapeutic, administering a second dose of the T cell activation therapeutic to the subject about three weeks after the first dose, and then administering the T cell activation therapeutic to the subject about once every eight weeks.

In some embodiments, step b) further comprises administering at least one additional therapeutic agent. In some embodiments, the at least one additional therapeutic agent is one or more of Rituximab, obinutuzumab, Cyclophosphamide, Mitoxantrone, Vincristine, Prednisone, etoposide, Methotrexate/Ifosfamide and Cytarabine, Dexamethasone, Cisplatin, Gemcitabine, Brentuximab vedotin, Bendamustine, liposomal Doxorubicin, Lenalidomide, ibrutinib, selinixor, Polatuzumab vedotin, Pixantrone, CAR-T cells (e.g. Yescarta, Kymriah), mozunetuzumab, bispecific antibodies, thalidomide, Autologous Stem Cell Transplant, or tafasitamab. In some embodiments, the at least one additional therapeutic agent is administered concurrently or sequentially with the inhibitor of PD-1 or PD-L1, T cell activation therapeutic and/or active agent.

In some embodiments, the hematologic malignancy is non-Hodgkin lymphoma (NHL). In some embodiments, the hematologic malignancy is diffuse large B cell lymphoma (DLBCL). In some embodiments, the DLBCL is a relapsed/refractory DLBCL. In some embodiments, the subject is a human.

### BRIEF DESCRIPTION OF THE DRAWINGS

FIG. 1 depicts the treatment regimen for the “SPiReL” Phase 2 clinical study.

FIG. 2 shows the baseline demographic information for enrolled participants (N=24).

FIG. 3 shows the baseline immune infiltrates, ELISpot and clinical responses. Nineteen subjects were included in the intent to treat (ITT) population and eleven subjects were evaluable in the per protocol (PP) population. Three participants had not reached evaluable time point. Immune infiltrates is shown for evaluable subjects with sample analysis completed. Positive for immune infiltrates was defined as >200 cells/mm². DPX-Survivac-induced T cell responses were observed in 8/15 (53.3%) subjects who had baseline sample and >2 different on-treatment samples available for analysis.

FIG. 4 shows survivin expression data from 33 screened participants analyzed centrally by immunohistochemistry (Rabbit Polyclonal antibody, Novus NB500-201). All 33 participants (100%) screened for enrollment were positive for survivin expression using a cutoff of expression at ≥10%. All enrolled subjects (n=22) demonstrated survivin expression at ≥70%. No trend between survivin expression and response was observed in this cohort.

FIG. 5 shows multiplex Immunohistochemistry (mIHC) analyses of the baseline (cells/mm²) of CD8+, CD4+, FoxP3+ and CD20+/PD-L1 in pre-treatment tumor samples (n=19) categorized sorted by subjects who achieved a clinical response (complete response (CR), partial response (PR), or stable disease (SD)) per Modified Cheson criteria (2007). One participant was evaluable but mIHC results were not obtained due to insufficient sample and are not included in this figure. For a participant to be identified as an ELISpot Responder, they will have had to exhibit a response to the DPX-Survivac peptide pool at least once on-treatment. For this analysis, subjects with a baseline sample and ≥2 different on-treatment samples are included. Analyses were performed at Akoya Biosciences using Opal Panel.

FIG. 6 shows the significance levels of the associations between different pairs of variables. The significances of the associations were obtained from univariate regression models between the variable pairs. The color scale reflects the significance of the association between variables. Only significant associations were plotted. The sample size was n=18 and the objective response categories included three “complete responses” (CR, nCR=3), three “partial responses” (PR, nPR=3), three “stable disease” (SD, nSD=3) and nine “progression disease” (PD, nPD=9). The “progression disease” (PD) category was comprised of seven “PD-clinical” and two “PD-radiology” responses. All immune infiltrates represents the combination of the following infiltrates: CD4+, CD8+, FoxP3+, CD4+ and FoxP3+, PD-L1.

FIGS. 7A-7B show the baseline immune cell infiltrates in the pre-treatment tumor biopsies from representative subjects with varying clinical responses. The upper panels show the spectral imaging for the designated antigen. The lower row of images show simulated immune histochemistry images for CD20-PD-L1. Analyses were performed at Akoya Biosciences using Opal Panel.

FIGS. 8A-8D show the PD-L1 expression for assessed subjects and statistical significance of the correlation of % PD-L1 positive cells with clinical response. FIGS. 8A and 8B represent percentage of PD-L1+CD20+ cells scored in the tumor region and categorized by observed clinical responses. FIGS. 8C and 8D represent percentage of total PD-L1+ cells scored in the tumor region and categorized by observed clinical responses. PD-L1 expression was assessed using mIHC analyses (Akoya Biosciences, Opal Panel) using CST #E1L3N antibody. PD-L1 expression across different clinical response groups were compared using two-tailed Mann-Whitney test. These figures show statistically significant correlation between presence of PD-L1+CD20+ cells in the tumor region or PD-L1+ cells in tumor region (any cells) and the clinical responses observed.

FIGS. 9A-9B show treatment induced Survivin T cell responses and that there is a correlation between the survivin-specific T cell response on treatment and the clinical responses observed. FIG. 9A demonstrates IFN-γ ELISpot responses represented as Spot Forming Units (SFU) per 10⁶ cells for longitudinally collected (baseline and on-treatment). The ELISpot responses are presented separately for subjects with CR, PR, SD and PD (per Modified Cheson Criteria 2007). FIG. 9B depicts pie-charts demonstrating the percentage of subjects with positive ELISpot responses within each of the clinical responders sub-groups. Subjects with a baseline sample and ≥2 different on-treatment samples are included for analysis (N=15). Positive response was defined as subjects with ≥1 on-treatment response (SFU per million-Background) greater than the cut-off. The cut-off was calculated as the mean of the peptide pool response at baseline+2SDEV.

FIGS. 10A-10B show treatment induced Survivin T cell responses. FIG. 10A depicts the time on treatment for all enrolled study participants (N=24) showing best overall response per Modified Cheson Criteria (2007) and separated as PD-L1+(defined as PD-L1 expression ≥10% by central mIHC, n=8), PD-L1 negative and subjects with PD-L1 status unknown. The ORR and DCR are described in the table (inset) for the FAS (N=23, 1 subject pending response). FIG. 10B depicts the best overall response, using the Modified Cheson Criteria, for evaluable Per Protocol (PP) subjects (N=14). PD-L1 positive subjects are shown by diagonal shading, defined as PD-L1 expression of ≥10% as assessed by central mIHC. The table (inset) demonstrates the ORR and DCR of the PP and in PD-L1+ subjects. One subject with a PR (11) did not have sufficient tissue to assess PD-L1 expression.

FIG. 11 shows the disease characteristics of subjects enrolled in the trial (N=19).

FIG. 12A provides a non-limiting example of an amino acid sequence encoding human survivin (SEQ ID NO: 1). FIG. 12B provides a coding sequence for a non-limiting example of survivin (Homo sapiens) (SEQ ID NO: 11) including stop codons.

FIGS. 13A-13B shows graphical representations of progression free survival (PFS) data. FIG. 13A shows a Kaplan Meier curve demonstrating PFS in the FAS (N=24). FIG. 13B shows a Kaplan Meier curve demonstrating PFS in subjects with positive baseline PD-L1 expression versus negative PD-L1 expression. PD-L1 positive is defined as expression ≥10% by central mIHC.

### DETAILED DESCRIPTION

The present application provides, among other things, methods of treating a hematologic malignancy (e.g., diffuse large B cell lymphoma (DLBCL)) in a subject in need thereof based on the detection of the expression of Programmed Death-Ligand 1 (PD-L1) in a biological sample of the subject. The methods also involve administering a combination treatment comprising a T cell activation therapeutic with an inhibitor of PD-L1 or Programmed Death 1 (PD-1). The methods may also involve the detection of the expression of PD-L1 in a biological sample of the subject in order to identify patients who are likely to benefit from the combination treatment comprising a T cell activation therapeutic with an inhibitor of PD-L1 or PD-1.

Despite the great potential of PD-1/PD-L1 blockade therapy in patients with select solid tumors and lymphomas, currently PD-1/PD-L1 blockade monotherapy has limited efficacy against DLBCL, particularly relapsed/refractory (r/r) DLBCL. For example, PD-1 blockade by nivolumab in patients with relapsed/refractory DLBCL in a phase II trial testing resulted in a low overall response rate. The objective response rates were 10% and 3%, respectively, in patients having failed or were ineligible for autologous transplantation (J Clin Oncol. 2019 Feb. 20; 37(6):481-489).

PD-L1 expression is reportedly highly diverse in DLBCL patients and can range from 25-70% (Young et al., Blood 2018, 131, 68-83). Such heterogeneity in the prevalence of PD-L1 expression might be the reason for the current disappointing low rate of clinical responses to PD-1/PD-L1 blockade therapy in DLBCL patients, as well as for the controversial prognostic significance of PD-L1 expression (Garcia-Lacarte et al., Cancers 2021, 13, 4683).

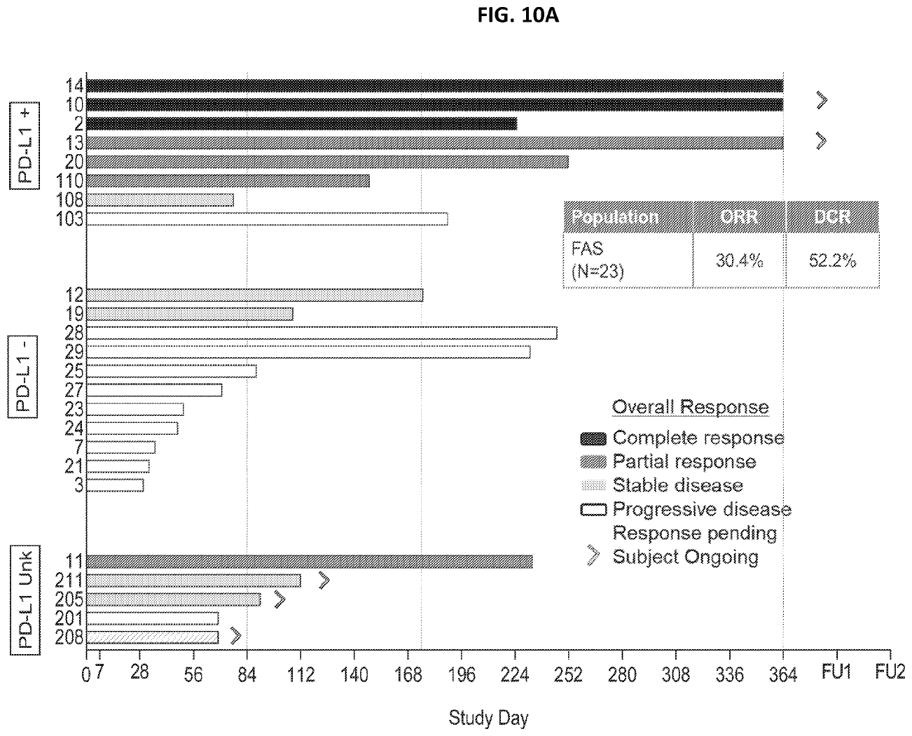

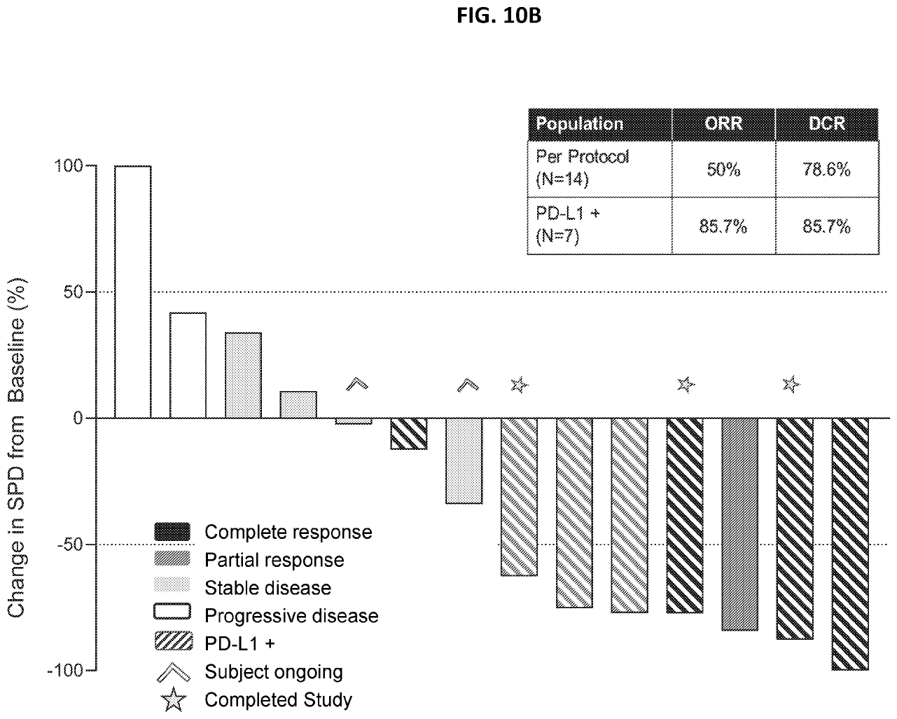

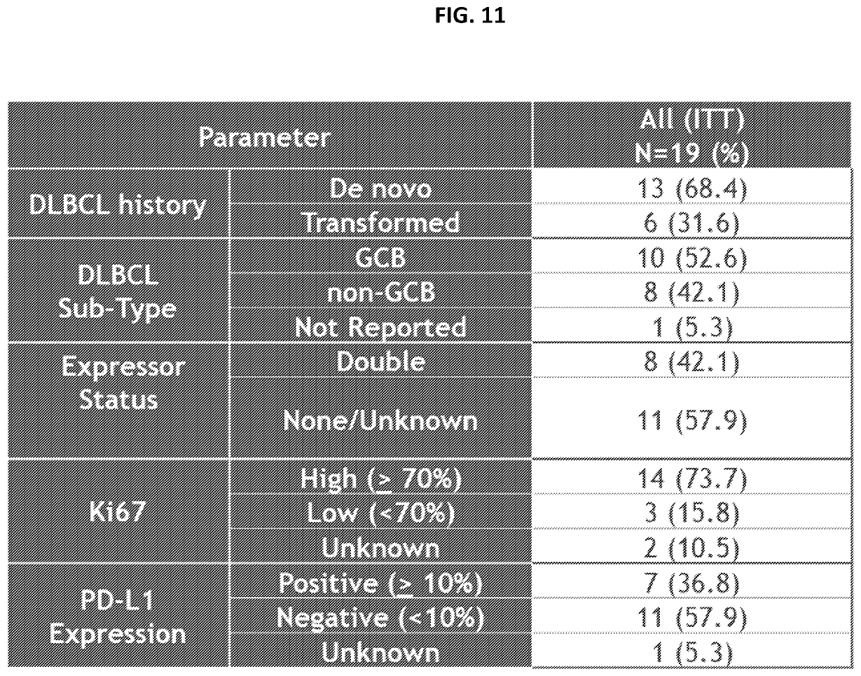

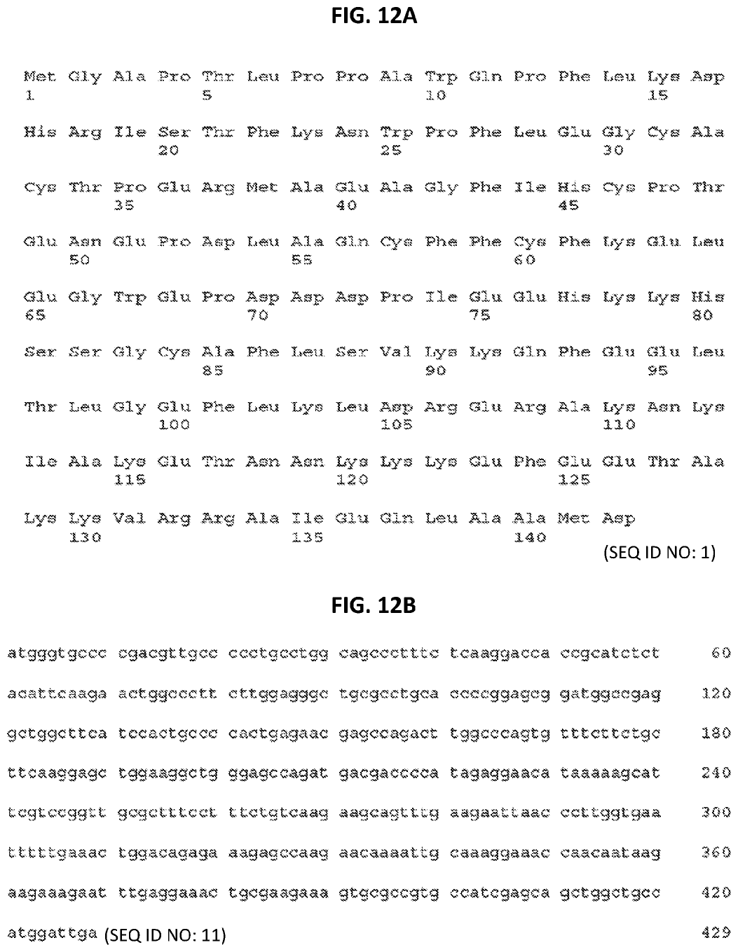

In the study presented the Examples section below, synergistic response was observed in r/r DLBCL patients receiving the combination treatment comprising a T cell activation therapeutic of the present disclosure with an inhibitor of PD-L1 or PD-1 of the present disclosure. Clinical responses are presented in FIGS. 10A and 10B. The clinical efficacy data showed surprisingly strong clinical response as compared to the historic Pembrolizumab monotherapy data in this setting.

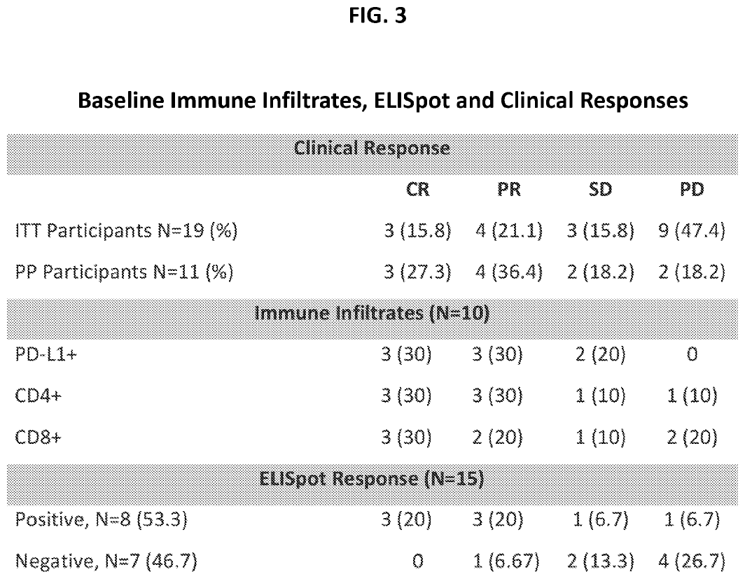

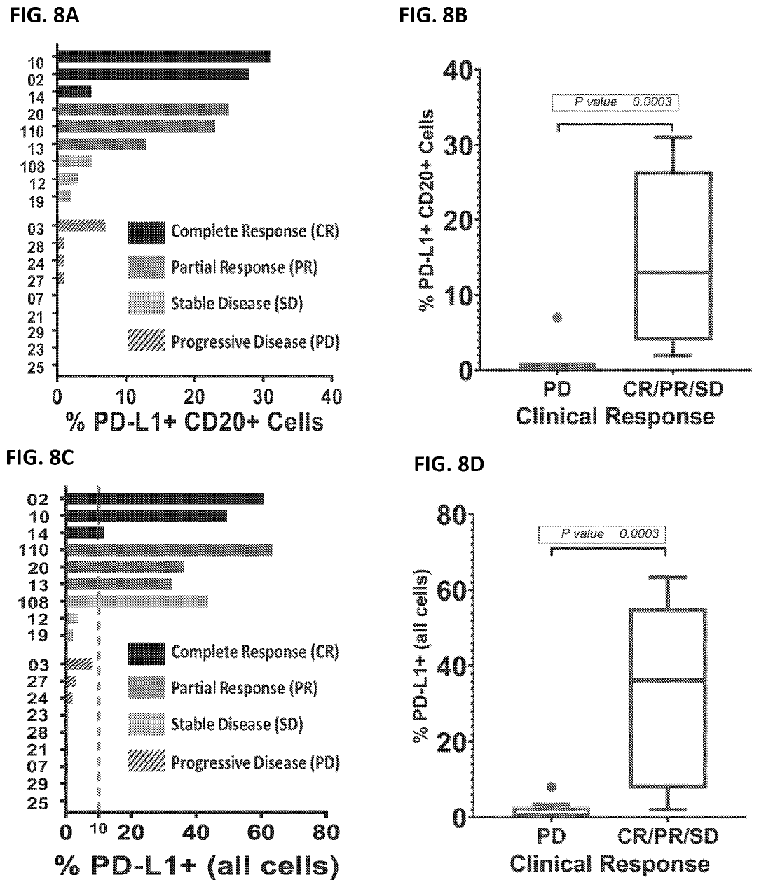

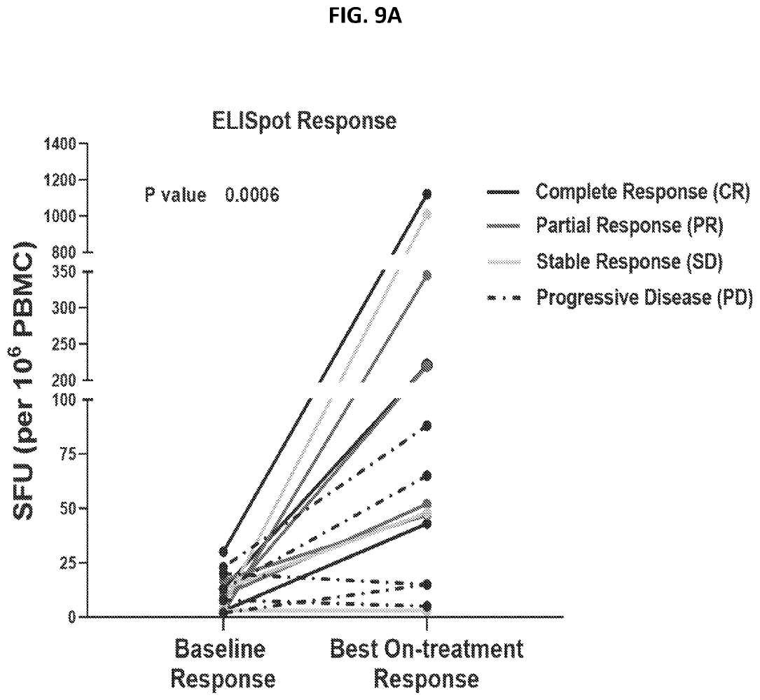

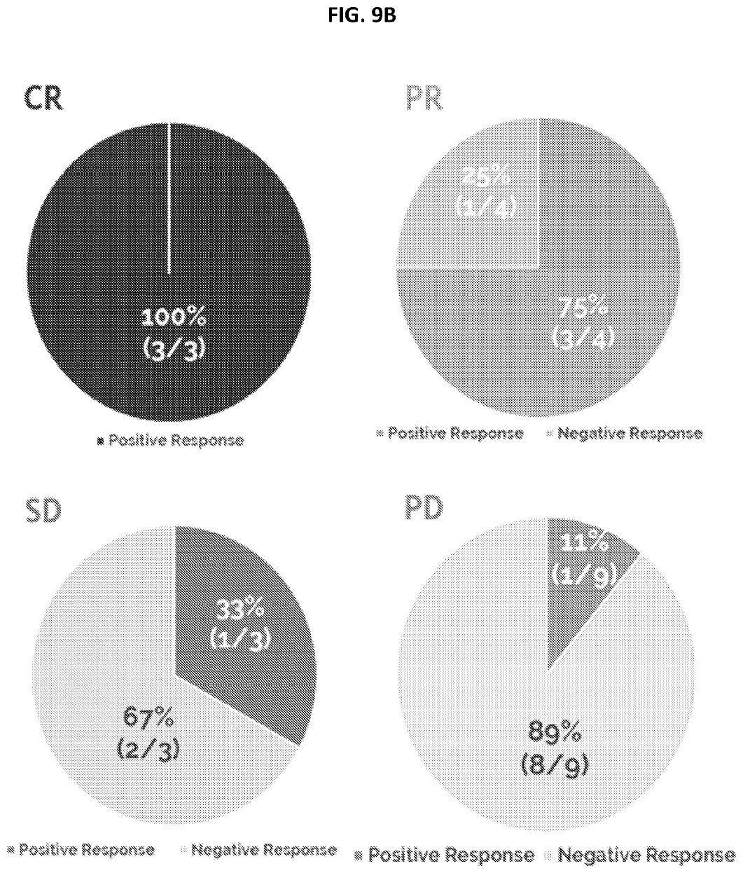

At the mechanism of action level, the synergistic effect is demonstrated in FIG. 3 whereby the majority of subjects with complete response (CR) and partial response (PR) were also positive for PD-L1 expression (pembrolizumab mechanism of action) as well as survivin-specific ELISpot responses on-treatment (DPX-Survivac mechanism of action). This data is further elaborated in FIGS. 8A-8D and 9A-9B.

Therefore, it was surprisingly found that the level of PD-L1 expression correlates with the clinical responses with the T cell activation therapeutic together with an inhibitor of PD-L1 or PD-1 in the treatment of hematologic malignancies, thus, making PD-L1 an unexpected biomarker for the treatment of DLBCL.

### Definitions

It must be noted that as used in this specification and the appended claims, the singular forms “a”, “an”, and “the” include plural reference unless the context clearly dictates otherwise. Unless defined otherwise all technical and scientific terms used herein have the same meaning as commonly understood to one of ordinary skill in the art to which this invention belongs.

The phrase “and/or”, as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used throughout herein, the term “about” means reasonably close. For example, “about” can mean within an acceptable standard deviation and/or an acceptable error range for the particular value as determined by one of ordinary skill in the art, which will depend on how the particular value is measured. Further, when whole numbers are represented, about can refer to decimal values on either side of the whole number. When used in the context of a range, the term “about” encompasses all of the exemplary values between the one particular value at one end of the range and the other particular value at the other end of the range, as well as reasonably close values beyond each end.

As used herein, whether in the specification or the appended claims, the transitional terms “comprising”, “including”, ‘carrying”, “having”, “containing”, “involving”, and the like are to be understood as being inclusive or open-ended (i.e., to mean including but not limited to), and they do not exclude unrecited elements, materials or method steps. Only the transitional phrases “consisting of” and “consisting essentially of”, respectively, are closed or semi-closed transitional phrases with respect to claims and exemplary embodiment paragraphs herein.

“Treating” or “treatment of”, or “preventing” or “prevention of”, as used herein, refers to an approach for obtaining beneficial or desired results. Beneficial or desired results can include, but are not limited to, alleviation or amelioration of one or more symptoms or conditions, diminishment of extent of disease, stabilisation of the state of disease, prevention of development of disease, prevention of spread of disease, delay or slowing of disease progression (e.g., suppression), delay or slowing of disease onset, conferring protective immunity against a disease-causing agent and amelioration or palliation of the disease state. “Treating” or “preventing” can also mean prolonging survival of a patient beyond that expected in the absence of treatment and can also mean inhibiting the progression of disease temporarily or preventing the occurrence of disease, such as by preventing infection in a subject. “Treating” or “preventing” may also refer to a reduction in the size of a tumor mass, reduction in tumor burden, reduction in target tumor burden, reduction in tumor aggressiveness, etc.

“Treating” may be distinguished from “preventing” in that “treating” typically occurs in a subject who already has a disease or disorder, or is known to have already been exposed to an infectious agent, whereas “preventing” typically occurs in a subject who does not have a disease or disorder, or is not known to have been exposed to an infectious agent. As will be appreciated, there may be overlap in treatment and prevention. For example, it is possible to be “treating” a disease in a subject, while at same time “preventing” symptoms or progression of the disease. Moreover, “treating” and “preventing” may overlap in that the treatment of a subject to induce an immune response (e.g., vaccination) may have the subsequent effect of preventing infection by a pathogen or preventing the underlying disease or symptoms caused by infection with the pathogen. These preventive aspects are encompassed herein by expressions such as “treatment of a tumor” or “treatment of cancer”.

As used herein, the terms “cancer”, “cancer cells”, “tumor”, and “tumor cells”, (used interchangeably) refer to cells that exhibit abnormal growth, characterized by a significant loss of control of cell proliferation or cells that have been immortalized. The term “cancer” or “tumor” includes metastatic as well as non-metastatic cancer or tumors. A cancer may be diagnosed using criteria generally accepted in the art, including the presence of a malignant tumor.

As used herein, a “therapeutically effective amount” means an amount of the inhibitor of PD-L1 or PD-1, T cell activation therapeutic, active agent, and/or any additional therapeutic effective to provide a therapeutic, prophylactic, or diagnostic benefit to a subject, and/or an amount sufficient to modulate an immune response and/or humoral response in a subject. As used herein, to “modulate” an immune and/or humoral response is distinct and different from activating an immune and/or humoral response. By “modulate”, it is meant that the active agent and/or additional therapeutic agent herein enhance an immune and/or humoral response that is activated by other mechanisms or compounds (e.g., by an antigen or immunogen). In an embodiment, the immune and/or humoral response was activated before the active agent, T cell activation therapeutic, and/or any additional therapeutic effective herein are administered. In another embodiment, the immune and/or humoral response may be activated commensurately to administration of the active agent, T cell activation therapeutic, and/or any additional therapeutic effective described herein. In another embodiment, the immune and/or humoral response may be activated subsequently to administration of the active agent, T cell activation therapeutic, and/or any additional therapeutic effective described herein.

The terms “subject”, “patient”, “individual”, and “animal” are used interchangeably herein and refer to mammals, including, without limitation, human and veterinary animals (e.g., primates, cats, dogs, cows, horses, sheep, pigs, rabbits, mice, rats, etc.) and experimental animal models. In a preferred embodiment, the subject is a human.

### Treatment Methods

In one aspect, provided herein is a method of treating a hematologic malignancy in a subject in need thereof, said method comprising
a) detecting the expression of Programmed Death-Ligand 1 (PD-L1, also known as B7-H1, CD274) in a biological sample of the subject; and
b) administering to the subject a therapeutically effective amount of an inhibitor of PD-L1 or Programmed Death 1 (PD-1, CD279), and a therapeutically effective amount of a T cell activation therapeutic, wherein PD-L1 expression is detected in the biological sample.

In another aspect, provided herein is a method of identifying a subject who is likely to benefit from a combination treatment comprising a T cell activation therapeutic with an inhibitor of PD-L1 or PD-1, wherein the subject has a hematologic malignancy, said method comprising
a) detecting the expression of PD-L1 in a biological sample of the subject; and
b) determining the subject as likely to benefit from a combination treatment comprising a T cell activation therapeutic with an inhibitor of PD-L1 or PD-1, wherein PD-L1 expression is detected in the biological sample.

The term “biological sample” as used herein includes any biological specimen obtained from a subject. Biological samples that can be used in the methods of the present disclosure include, without limitation, tumor sample, whole blood, plasma, serum, red blood cells, white blood cells (e.g., peripheral blood mononuclear cells (PBMC), polymorphonuclear (PMN) cells), ductal lavage fluid, nipple aspirate, lymph (e.g., disseminated tumor cells of the lymph node), bone marrow aspirate, saliva, urine, stool (i.e., feces), sputum, bronchial lavage fluid, fine needle aspirate (e.g., harvested by random periareolar fine needle aspiration), any other bodily fluid, a tissue sample such as a biopsy of a site of inflammation (e.g., needle biopsy), and cellular extracts thereof. In some embodiments, the biological sample is a tumor sample. In some embodiments, the biological sample is a tumor biopsy sample.

In some embodiments, the methods described herein further comprise obtaining a biological sample from the subject prior to the detecting step.

In some embodiments, in step b) PD-L1 expression is detected in at least one cell in the biological sample. In some embodiments, PD-L1 expression is detected in at least 0.1%, at least 0.5%, at least 1%, at least 1.5%, at least 2%, at least 2.5%, at least 3%, at least 3.5%, at least 4%, at least 4.5%, at least 5%, at least 5.5%, at least 6%, at least 6.5%, at least 7%, at least 7.5%, at least 8%, at least 8.5%, at least 9%, at least 9.5%, at least 10%, at least 11, at least 12%, at least 13%, at least 14%, at least 15%, at least 16%, at least 17%, at least 18%, at least 19%, at least 20%, at least 21%, at least 22%, at least 23%, at least 24%, at least 25%, at least 16%, at least 27%, at least 28%, at least 29%, at least 30%, at least 35%, at least 40%, at least 45%, at least 50%, or above 50% of the cells in the biological sample. In some embodiments, PD-L1 expression is detected in about 1% to 3%, about 2% to 4%, about 1% to 5%, about 3% to 5%, about 4% to 6%, about 7% to 9%, about 8% to 10%, about 5% to 10%, about 9% to 11%, about 10% to 12%, about 11% to 13%, about 12% to 14%, about 13% to 15%, about 10% to 15%, about 14% to 16%, about 15% to 17%, about 16% to 18%, about 17% to 19%, about 18% to 20%, about 15% to 20%, about 20% to 25%, about 25% to 30%, about 30% to 35%, about 35% to 40%, about 40% to 45%, or about 45% to 50% of the cells in the biological sample. In some embodiments, PD-L1 expression is detected in about 1% to 10%, about 1% to about 15%, about 1% to about 20%, about 1% to about 25%, about 1% to about 30%, about 1% to about 35%, about 1% to about 40%, about 1% to about 45%, about 1% to about 50%, about 5% to 10%, about 5% to about 15%, about 5% to about 20%, about 5% to about 25%, about 5% to about 30%, about 5% to about 35%, about 5% to about 40%, about 5% to about 45%, about 5% to about 50%, about 10% to about 15%, about 10% to about 20%, about 10% to about 25%, about 10% to about 30%, about 10% to about 35%, about 10% to about 40%, about 10% to about 45%, about 10% to about 50%, about 20% to about 25%, about 20% to about 30%, about 20% to about 35%, about 20% to about 40%, about 20% to about 45%, about 20% to about 50%, about 30% to about 35%, about 30% to about 40%, about 30% to about 45%, about 30% to about 50%, about 40% to about 45%, or about 40% to about 50% of the cells in the biological sample. In some embodiments, PD-L1 expression is detected in at least about 50% of the cells in the biological sample.

In some embodiments, the methods described herein involve detecting PD-L1 expression in any cells in the biological sample (e.g., tumor or non-tumor cells). In some embodiments, the methods involve detecting PD-L1 expression in tumor cells, lymphocytes and/or macrophages. In some embodiments, the methods involve detecting PD-L1 expression in tumor cells. In some embodiments, the methods involve detecting PD-L1 expression in CD20+ cells.

In some embodiments, in step b) PD-L1 expression is detected in at least one tumor cell in the biological sample. In some embodiments, PD-L1 expression is detected in at least 0.1%, at least 0.5%, at least 1%, at least 1.5%, at least 2%, at least 2.5%, at least 3%, at least 3.5%, at least 4%, at least 4.5%, at least 5%, at least 5.5%, at least 6%, at least 6.5%, at least 7%, at least 7.5%, at least 8%, at least 8.5%, at least 9%, at least 9.5%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 16%, at least 27%, at least 28%, at least 29%, at least 30%, at least 35%, at least 40%, at least 45%, at least 50%, or above 50% of the tumor cells in the biological sample. In some embodiments, PD-L1 expression is detected in about 1% to 3%, about 2% to 4%, about 1% to 5%, about 3% to 5%, about 4% to 6%, about 7% to 9%, about 8% to 10%, about 5% to 10%, about 9% to 11%, about 10% to 12%, about 11% to 13%, about 12% to 14%, about 13% to 15%, about 10% to 15%, about 14% to 16%, about 15% to 17%, about 16% to 18%, about 17% to 19%, about 18% to 20%, about 15% to 20%, about 20% to 25%, about 25% to 30%, about 30% to 35%, about 35% to 40%, about 40% to 45%, or about 45% to 50% of the tumor cells in the biological sample. In some embodiments, PD-L1 expression is detected in about 1% to 10%, about 1% to about 15%, about 1% to about 20%, about 1% to about 25%, about 1% to about 30%, about 1% to about 35%, about 1% to about 40%, about 1% to about 45%, about 1% to about 50%, about 5% to 10%, about 5% to about 15%, about 5% to about 20%, about 5% to about 25%, about 5% to about 30%, about 5% to about 35%, about 5% to about 40%, about 5% to about 45%, about 5% to about 50%, about 10% to about 15%, about 10% to about 20%, about 10% to about 25%, about 10% to about 30%, about 10% to about 35%, about 10% to about 40%, about 10% to about 45%, about 10% to about 50%, about 20% to about 25%, about 20% to about 30%, about 20% to about 35%, about 20% to about 40%, about 20% to about 45%, about 20% to about 50%, about 30% to about 35%, about 30% to about 40%, about 30% to about 45%, about 30% to about 50%, about 40% to about 45%, or about 40% to about 50% of the tumor cells in the biological sample. In some embodiments, PD-L1 expression is detected in at least about 50% of the tumor cells in the biological sample.

In some embodiments, the method of treating a hematologic malignancy described herein may comprise not administering to the subject a therapeutically effective amount of an inhibitor of PD-L1 or PD-1, and a therapeutically effective amount of a T cell activation therapeutic, wherein PD-L1 expression is not detected in the biological sample. In some embodiments, the method of treating a hematologic malignancy described herein may comprise not administering to the subject a therapeutically effective amount of an inhibitor of PD-L1 or PD-1, and a therapeutically effective amount of a T cell activation therapeutic, wherein PD-L1 expression is lower than a threshold value, e.g., 1%, 5%, or 10% of the cells in the biological sample; or 1%, 5%, or 10% of the tumor cells in the biological sample.

In some embodiments, the method of identifying a subject who is likely to benefit from a combination treatment described herein may comprise determining the subject as unlikely to benefit from a combination treatment comprising a T cell activation therapeutic with an inhibitor of PD-L1 or PD-1, wherein PD-L1 expression is detected in the biological sample. In some embodiments, the method of identifying a subject who is likely to benefit from a combination treatment described herein may comprise determining the subject as unlikely to benefit from a combination treatment comprising a T cell activation therapeutic with an inhibitor of PD-L1 or PD-1, wherein PD-L1 expression is lower than a threshold value, e.g., 1%, 5%, or 10% of the cells in the biological sample; or 1%, 5%, or 10% of the tumor cells in the biological sample.

Detection of PD-L1 in a biological sample (e.g., tumor sample) may be performed using techniques described herein and/or those known in the art. Non-limiting examples of techniques that may be used in the methods described herein include a multiplex immunofluorescence (mIF) assay, an immunohistochemistry (IHC) assay, a fluorescence in situ hybridization (FISH) assay, RNAscope, or flow cytometry. Multiplex immunofluorescence (mIF) assays may be carried out as described herein or in, for example, Lee et al., J Immunol Methods. 2020 March; 478:112714. doi: 10.1016/j.jim.2019.112714, which is incorporated herein by reference in its entirety for all purposes. One non-limiting example of a multiplex immunofluorescence (mIF) assay involves the use of a rabbit monoclonal anti-human PD-L1 antibody, clone E1L3N for detection of PD-L1. Immunohistochemistry (IHC), RNAscope, or fluorescence in situ hybridization (FISH) may be carried out as described in, for example, Huang et al., Cancer Med. 2019; 8(8):3831-3845. doi:10.1002/cam4.2316, which is incorporated herein by reference in its entirety for all purposes. One non-limiting example of an IHC assay involves the use of a monoclonal mouse anti-PD-L1 antibody, Clone 22C3 for detection of PD-L1 in formalin-fixed, paraffin-embedded (FFPE) tissues.

In another aspect, provided herein is a method of improving efficacy of a PD-L1 or PD-1 blockade treatment in a subject having a hematologic malignancy, wherein the subject is undergoing treatment with an inhibitor of PD-L1 or PD-1, said method comprising
a) detecting the expression of PD-L1 in a biological sample of the subject; and
b) administering a therapeutically effective amount of a T cell activation therapeutic, wherein PD-L1 expression is detected in at least one cell in the biological sample.

In certain embodiments, the methods disclosed herein may further comprise detecting the level of CD4+ and/or CD8+ infiltration in the biological sample. In some embodiments, the methods disclosed herein involve administering a therapeutically effective amount of an inhibitor of PD-L1 or PD-1, and a therapeutically effective amount of a T cell activation therapeutic, wherein higher level of CD4+ and/or CD8+ infiltration is detected in the biological sample. In some embodiments, higher level of CD4+ infiltration refers to the presence of CD4+ cells in at least 10 cells/mm², at least 15 cells/mm², at least 20 cells/mm², at least 25 cells/mm², at least 30 cells/mm², at least 40 cells/mm², at least 50 cells/mm², at least 60 cells/mm², at least 70 cells/mm², at least 75 cells/mm², at least 80 cells/mm², at least 90 cells/mm², 100 cells/mm², at least 200 cells/mm², at least 300 cells/mm², at least 400 cells/mm², at least 500 cells/mm², at least 600 cells/mm², at least 700 cells/mm², at least 800 cells/mm², at least 900 cells/mm², at least 1000 cells/mm², at least 1100 cells/mm², at least 1200 cells/mm², at least 1300 cells/mm², at least 1400 cells/mm², at least 1500 cells/mm², at least 1600 cells/mm², at least 1700 cells/mm², at least 1800 cells/mm², at least 1900 cells/mm², at least 2000 cells/mm², at least 2500 cells/mm², at least 3000 cells/mm², at least 3500 cells/mm², at least 4000 cells/mm² or more than 4000 cells/mm² in the biological sample. In some embodiments, higher level of CD8+ infiltration refers to the presence of CD8+ cells in at least 100 cells/mm², at least 200 cells/mm², at least 300 cells/mm², at least 400 cells/mm², at least 500 cells/mm², at least 600 cells/mm², at least 700 cells/mm², at least 800 cells/mm², at least 900 cells/mm², at least 1000 cells/mm², at least 1100 cells/mm², at least 1200 cells/mm², at least 1300 cells/mm², at least 1400 cells/mm², at least 1500 cells/mm², at least 1600 cells/mm², at least 1700 cells/mm², at least 1800 cells/mm², at least 1900 cells/mm², at least 2000 cells/mm², at least 2500 cells/mm², at least 3000 cells/mm², at least 3500 cells/mm², at least 4000 cells/mm² or more than 4000 cells/mm² in the biological sample. In some embodiments, the biological sample is a tumor sample.

In certain embodiments, the methods disclosed herein comprise administering an inhibitor of PD-1 or PD-L1 and a T cell activation therapeutic (e.g., T cell activation therapeutic comprising at least one survivin antigen (e.g., DPX-Survivac)) to a subject for treating a hematologic malignancy. In certain embodiments, the methods further comprise administering one or more active agent. In yet further embodiments, the methods may comprise administering at least one additional therapeutic agent.

In certain embodiments, the inhibitor of PD-1 or PD-L1, the active agent and additional therapeutic agent are administered with the same regimen. In certain embodiments, the inhibitor of PD-1 or PD-L1, active agent and additional therapeutic agent are administered with different regimens.

An inhibitor of PD-1 or PD-L1, T cell activation therapeutic, active agent and/or additional therapeutic agent as disclosed herein may be administered to a subject in a therapeutically effect amount. In certain embodiments, the effective amount of the inhibitor of PD-1 or PD-L1, T cell activation therapeutic, active agent and/or additional therapeutic agent is an amount sufficient to provide an immune-modulating effect.

The term “agent” includes any substance, molecule, element, compound, entity, or a combination thereof. It can be a natural product, a synthetic compound, or a combination of two or more substances. Unless otherwise specified, the terms “agent”, “substance”, and “compound” are used interchangeably herein.

As used herein, an “active agent” or “additional therapeutic agent” refers to a pharmaceutically or therapeutically agent. The active agent and/or additional therapeutic agent can each individually be a small molecule drug, an antibody, an antibody mimetic, or a functional equivalent or functional fragment of any one thereof.

The active agent used in the methods of the present disclosure may improve the efficacy of the T cell activation therapeutic. As used herein, “improving T cell activation therapeutic efficacy” or “improving the efficacy of T cell activation therapeutic” or the like refers to any change or alteration in the immune response of a subject that is capable of rendering the T cell activation therapeutic (e.g., survivin therapeutic) of the disclosure more effective in the treatment of cancer. In some embodiments, this may involve accelerating the appearance of an immune response and/or improving the persistence or strength of an immune response to the T cell activation therapeutic (e.g., survivin therapeutic). The immune response may either be a cell-mediated immune response or a humoral immune response or a combination of both.

In the methods of the disclosure, an agent may “improve the efficacy of the T cell activation therapeutic” (e.g., survivin therapeutic) by either directly or indirectly enhancing the immune response against the survivin antigen in the T cell activation therapeutic. This may be accomplished, for example, by reducing the number and/or activity of suppressive immune cells. It has been reported that the tumor microenvironment, for example, upregulates many factors that promote the development of suppressive immune cells, such as CD4+FoxP3+ regulatory T cells (Tregs) (Curiel et al., Nat Med 10(9): 942-949, 2004), myeloid-derived suppressor cells (MDSCs) (Nagaraj and Gabrilovich, Cancer Res 68(8): 2561-3, 2008), and CD19+CD5+CD1dhiIL-10+ B cells (Bregs) (Balkwill et al., Trends Immunol, 2013 April; 34(4):169-73). Therefore, the ability to reduce the number or activity of these suppressive immune cells represents an embodiment for improving T cell activation therapeutic efficacy.

“Improving the efficacy of a T cell activation therapeutic” (e.g., survivin therapeutic) may also be accomplished, for example, by increasing the number and/or activity of antigen-specific CD8+ T cells. In this regard, it has been reported that the tumor microenvironment, for example, contributes to the direct suppression of activated CD8+ T cells by releasing immunosuppressive cytokines such as TNF-α and TGF-β (Yang et al., Trends Immunol 31(6): 220-227, 2010). Therefore, the ability to increase the activity of antigen-specific CD8+ T cells represents a potential mechanism of improving T cell activation therapeutic efficacy. An increase in antigen-specific CD8+ T cells may be the result of an increased number of such cells, increased activity or such cells, and/or the generation of an enriched population of antigen-specific CD8+ T cells relative to total CD8+ T cells, such as for example by a relative decrease in total CD8+ T cells.

More generally, “improving the efficacy of a T cell activation therapeutic” refers to the ability of the methods of the disclosure to enhance the immunogenicity of the T cell activation therapeutic (e.g., survivin therapeutic), by enhancing a cell-mediated immune response and/or humoral immune response induced by the survivin therapeutic; increase the number of immune cells and/or antibodies at a site of vaccination or a tumor site; or improve a therapeutic effect provided by the T cell activation therapeutic (e.g., survivin therapeutic) of the disclosure, such as by enhancing the prophylactic and/or therapeutic treatment of cancer and/or alleviating, delaying or inhibiting the progression of disease symptoms. Improving the efficacy of a T cell activation therapeutic (e.g., survivin therapeutic) may also be associated with an improved quality of life or a decreased morbidity, as compared with monotherapy treatment.

“Improving the efficacy of a T cell activation therapeutic” may also mean that lower doses of the active ingredients of the combination of the disclosure are needed to produce the desired result. This encompasses both embodiments where the dosages themselves are smaller and embodiments where the T cell activation therapeutic (e.g., survivin therapeutic), active agent and/or additional therapeutic agent (e.g., one that interferes with DNA replication and/or an immunomodulatory agent), are applied less frequently.

In the methods disclosed herein, the amount of any specific inhibitor of PD-1 or PD-L1, a T cell activation therapeutic, active agent and/or additional therapeutic agent may depend on the type of agent, the disease or disorder to be treated, and/or particular characteristics of the subject (e.g., age, weight, sex, immune status, etc.). One skilled in the art can readily determine the amount of active agent and/or additional therapeutic agent needed in a particular application by empirical testing.

T Cell Activation Therapeutic Compositions

In various embodiments of the present disclosure, the T cell activation therapeutic comprises at least one survivin antigen. T cell activation therapeutic compositions of the disclosure may be of any form suitable for delivery of a survivin antigen to a subject. T cell activation therapeutic compositions according to the disclosure can be formulated according to known methods, such as by admixture of the one or more survivin antigens with one or more pharmaceutically acceptable excipients or carriers, preferably those acceptable for administration to humans. Examples of such excipients, carriers and methods of formulation may be found e.g., in Remington's Pharmaceutical Sciences (Maack Publishing Co, Easton, PA). To formulate a pharmaceutically acceptable T cell activation therapeutic composition suitable for effective administration, such compositions will typically contain a therapeutically effective amount of a survivin antigen, such as a survivin polypeptide, a survivin peptide, or a survivin peptide variant as described herein, or a nucleic acid molecule or vector encoding such survivin antigen.

The term “antigen” includes any substance, drug, molecule, element, compound, or combination thereof that is intended to be delivered to a subject. An antigen may be incorporated into a composition of the present invention as a hydrophobic phase antigen if it is contained in the hydrophobic phase of the composition, or as a hydrophilic phase antigen if it is contained in the hydrophilic phase of the composition. An antigen can be a natural product, a synthetic compound, or a combination of two or more substances. An antigen may be a peptide antigen, a DNA polynucleotide encoding an antigen; or an RNA polynucleotide encoding an antigen, or a functional equivalent or functional fragment of any one thereof. In some embodiments, the antigen is a DNA polynucleotide or an RNA polynucleotide encoding an antigen. In some embodiments, the antigen is a peptide antigen. In some embodiments, the peptide antigen is glycosylated.

T cell activation therapeutic compositions according to the disclosure may be administered to a subject in a therapeutically effect amount. As used herein, a “therapeutically effective amount” means an amount T cell activation therapeutic or active ingredient (e.g., one or more survivin antigens) effective to treat, prevent, alleviate, or ameliorate a tumor or cancer or symptoms of a tumor or cancer (e.g., a hematologic malignancy); prolong the survival of the subject being treated; and/or stimulate, induce or enhance an immune response in a subject, such as a cytotoxic T cell response. In some embodiments, a therapeutically effective amount of the T cell activation therapeutic is an amount capable of inducing a clinical response in a subject in the treatment of a tumor. Determination of a therapeutically effective amount of the T cell activation therapeutic is well within the capability of those skilled in the art, especially in light of the disclosure provided herein. The therapeutically effective amount may vary according to a variety of factors such as the subject's condition, weight, sex and age.

Once one or more appropriate survivin antigens have been selected for inclusion in a T cell activation therapeutic composition according to the disclosure, the antigens may be delivered by various suitable means which are known in the art. T cell activation therapeutic compositions for use in the methods described herein can include for example, and without limitation, lipopeptides (e.g., Vitiello, A. et al., J. Clin. Invest. 95:341, 1995), peptide compositions encapsulated in poly(DL-lactide-co-glycolide) (“PLG”) microspheres (see, e.g., Eldridge, et al., Molec. Immunol. 28:287-294, 1991; Alonso et al., Vaccine 12:299-306, 1994; Jones et al., Vaccine 13:675-681, 1995), peptide compositions contained in immune stimulating complexes (ISCOMS) (see, e.g., Takahashi et al., Nature 344:873-875, 1990; Hu et al., Clin Exp Immunol. 113:235-243, 1998), multiple antigen peptide systems (MAPs) (see e.g., Tam, J. P., Proc. Natl. Acad. Sci. U.S.A. 85:5409-5413, 1988; Tam, J. P., J. Immunol. Methods 196:17-32, 1996), peptides formulated as multivalent peptides; peptides for use in ballistic delivery systems, typically crystallized peptides, viral delivery vectors (Perkus, M. E. et al., In: Concepts in vaccine development, Kaufmann, S. H. E., ed., p. 379, 1996; Chakrabarti, S. et al., Nature 320:535, 1986; Hu, S. L. et al., Nature 320:537, 1986; Kieny, M.-P. et al., AIDS Bio/Technology 4:790, 1986; Top, F. H. et al., J. Infect. Dis. 124:148, 1971; Chanda, P. K. et al., Virology 175:535, 1990), particles of viral or synthetic origin (e.g., Kofler, N. et al., J. Immunol. Methods. 192:25, 1996; Eldridge, J. H. et al., Sem. Hematol. 30:16, 1993; Falo, L. D., Jr. et al., Nature Med. 7:649, 1995), adjuvants (Warren, H. S., Vogel, F. R., and Chedid, L. A. Annu. Rev. Immunol. 4:369,1986; Gupta, R. K. et al., Vaccine 11:293, 1993), liposomes (Reddy, R. et al, J. Immunol. 148:1585, 1992; Rock, K. L, Immunol. Today 17:131, 1996), or, naked or particle absorbed cDNA (Ulmer, J. B. et al., Science 259:1745, 1993; Robinson, H. L, Hunt, L. A., and Webster, R. G., Vaccine 11:957, 1993; Shiver, J. W. et al., In: Concepts in vaccine development, Kaufmann, S. H. E., ed., p. 423, 1996; Cease, K. B., and Berzofsky, J. A., Annu. Rev. Immunol. 12:923, 1994 and Eldridge, J. H. et al., Sem. Hematol. 30:16, 1993). Each reference disclosed in this paragraph is incorporated herein by reference for all intended purposes.

T cell activation therapeutic compositions of the disclosure also encompass nucleic acid mediated modalities. For example, DNA or RNA encoding one or more of the survivin antigens as described herein may be administered to the subject. Such approaches are described, for example, in Wolff et al., Science 247:1465 (1990) as well as U.S. Pat. Nos. 5,580,859; 5,589,466; 5,804,566; 5,739, 118; 5,736,524; 5,679,647; and WO 98/04720. Examples of DNA-based delivery technologies include “naked DNA”, facilitated (bupivicaine, polymers, peptide-mediated) delivery, cationic lipid complexes, and particle-mediated (“gene gun”) or pressure-mediated delivery (see, e.g., U.S. Pat. No. 5,922,687). Each reference disclosed in this paragraph is incorporated herein by herein for all intended purposes.

In further embodiments of the T cell activation therapeutic compositions, the survivin antigens (e.g., survivin peptides) may also be expressed by viral or bacterial vectors. Examples of expression vectors include attenuated viral hosts, such as vaccinia or fowlpox. This approach involves the use of vaccinia virus, for example, as a vector to express nucleotide sequences that encode the survivin peptides as described herein. Upon introduction into an acutely or chronically infected host or into a non-infected host, the recombinant vaccinia virus expresses the antigenic peptide, and thereby elicits a host immune response. Vaccinia vectors and methods useful in immunization protocols are described in, e.g., U.S. Pat. No. 4,722,848. Another vector is BCG (Bacille Calmette Guerin). BCG vectors are described in Stover et al., Nature 351:456-460 (1991). A wide variety of other vectors useful for therapeutic administration or immunization of the peptides of the disclosure, e.g., adeno and adeno-associated virus vectors, retroviral vectors, Salmonella typhi vectors, detoxified anthrax toxin vectors, and the like, will be apparent to those skilled in the art and are encompassed by the T cell activation therapeutic compositions described herein. Each reference disclosed in this paragraph is incorporated by reference herein for all intended purposes.

A T cell activation therapeutic in accordance with the disclosure also encompasses compositions containing one or more of the survivin antigens, where the antigen can be present individually or as a construct containing multiple copies of the same or different survivin antigens. For example, the survivin antigen can be present as a single nucleic acid molecule (e.g., vector) encoding several of the same or different survivin antigens. Or, in other embodiments, a homopolymer comprising multiple copies of the same survivin antigen, or a heteropolymer of various different survivin antigens, may be used. Such polymers may have the advantage of providing an increased immunological reaction as they comprise multiple copies of survivin antigens, such that the resultant effect may be an enhanced ability to induce an immune response with the one or more antigenic determinants of survivin. The composition can comprise a naturally occurring region of one or more survivin antigens or can comprise prepared antigens, e.g., recombinantly or by chemical synthesis.

A T cell activation therapeutic of the disclosure can also include antigen-presenting cells (APC), such as dendritic cells (DC), as a vehicle to present the one or more survivin antigens (e.g., survivin peptides). Such T cell activation therapeutic compositions can be created in vitro, following dendritic cell mobilization and harvesting, whereby loading of dendritic cells occurs in vitro. For example, dendritic cells are transfected with DNA or RNA encoding the one of more survivin antigens or are pulsed with survivin peptide antigens. The dendritic cell can then be administered to a subject to elicit an immune response in vivo.

A T cell activation therapeutic according to the disclosure may be administered by any suitable means, such as e.g., injection (e.g., intramuscular, intradermal, subcutaneous, intravenous or intraperitoneal), aerosol, oral, nasal, topical, intravaginal, transdermal, transmucosal, or any other suitable routes. The T cell activation therapeutic may be formulated for systemic or localized distribution in the body of the subject. Systemic formulations include those designed for administration by injection, as well as those designed for transdermal, transmucosal or oral administration.

For injection, the T cell activation therapeutics may be formulated in a carrier comprising a continuous phase of a hydrophobic substance as described herein, such as a water-in-oil emulsion or an oil-based carrier. In some embodiments, lipid vesicle particles (e.g., liposomes) may be used together with the carrier. The T cell activation therapeutics may also be formulated as aqueous solutions such as in Hank's solution, Ringer's solution or physiological saline buffer.

As will be apparent from the above, T cell activation therapeutic compositions of the disclosure are meant to encompass any composition or antigen delivery means (e.g., viral vectors, viral like particles, lipid vesicle particles, etc.) which are useful in the treatment of cancer, including compositions capable of stimulating an immune response in a subject, such as a specific cytotoxic T cell response upon administration. In some embodiments, the lipid vesicle particles used is a bilayer vesicular structure, such as for example, a liposome. Lipid vesicle particles are completely closed lipid bilayer membranes containing an entrapped aqueous volume. Lipid vesicle particles may be unilamellar vesicles (possessing a single bilayer membrane) or multilamellar vesicles characterized by multimembrane bilayers, each bilayer may or may not be separated from the next by an aqueous layer. A general discussion of liposomes can be found in Gregoriadis 1990; and Frezard 1999. Lipid vesicle particles can adsorb to virtually any type of cell and then release an incorporated agent (e.g., Survivin antigens). Alternatively, the lipid vesicle particles can fuse with the target cell, whereby the contents of the lipid vesicle particles empty into the target cell. Alternatively, a lipid vesicle particle may be endocytosed by cells that are phagocytic. Lipid vesicle particles have been used in the preparation of compositions comprising a hydrophobic carrier as a vesicle to encapsulate antigens as well as an emulsifier to stabilize the formulation (see e.g., WO2002/038175, WO2007/041832, WO2009/039628, WO2009/146523 and WO2013/049941, each of which is hereby incorporated by reference in its entirety). Hydrophilic antigens are typically entrapped in the hydrophilic interior, while hydrophobic antigens can be intercalated in the lipid bilayer or dispersed in the oil phase. In another embodiment, pre-manufactured lipid vesicle particles may be used in the vaccine compositions disclosed herein. In embodiments where the composition is water-free, one or more of the components of the composition (e.g., Survivin, adjuvant, and/or T-helper epitope) may be encapsulated in, or mixed or suspended with, lipid vesicle particles in a hydrophilic phase; lyophilized; and then reconstituted in the hydrophobic carrier. In such embodiments, the lipid vesicle particles may reorganize to form alternate structures in the hydrophobic carrier.

To obtain T cell activation therapeutic compositions of the invention, it may be suitable to combine the survivin antigen, which may be a relatively small survivin peptide, with various materials such as adjuvants, excipients, surfactants, immunostimulatory components and/or carriers. In certain embodiments, the peptides can be about 8 about 24 amino acids in length. In certain embodiments, the peptides can be about 8 to about 11 amino acids in length. In certain embodiments, the peptides can be about 15 to about 24 amino acids in length. In certain embodiments, the peptide can be 5 to 120 amino acids in length, 5 to 100 amino acids in length, 5 to 75 amino acids in length, 5 to 50 amino acids in length, 5 to 40 amino acids in length, 5 to 30 amino acids in length, 5 to 20 amino acids in length or 5 to 10 amino acids in length. In certain embodiments, the peptide antigen can be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acids in length. Adjuvants may be included in the T cell activation therapeutic composition to enhance the specific immune response. Different carriers may be used depending on the desired route of administration or the desired distribution in the subject, e.g., systemic or localized.

In a particular embodiment, the T cell activation therapeutic composition for use in the methods of the invention is a composition comprising at least one survivin antigen, lipid vesicle particles and a carrier comprising a continuous phase of a hydrophobic substance (e.g., Mineral oil, Incomplete Freund's Adjuvant (IFA), Montanide® ISA 51, VG) to form or reorganize to form alternate structures (e.g., reverse micelles) in the hydrophobic carrier. In a further embodiment, the composition may additionally comprise an adjuvant. In a further embodiment, the composition may additionally comprise a T-helper epitope or antigen.

Thus, in an embodiment, the T cell activation therapeutic composition comprises one or more survivin antigens; a T-helper epitope; an adjuvant; lipid vesicle particles; and a carrier comprising a continuous phase of a hydrophobic substance. The T-helper epitope may, for example, be a peptide comprising the amino acid sequence AQYIKANSKFIGITEL (SEQ ID NO: 10). The adjuvant may be, by way of example and not limitation, a polyL:C poly dIdC polynucleotide.

In a further embodiment, the T cell activation therapeutic composition for use in the methods of the invention is a composition comprising at least one survivin antigen, together with a lipid vesicle particle-based and/or amphipathic compound-based vaccine adjuvanting platform, including, but not limited to, the VacciMax®, DepoVax™, and DPX™ platform technologies (see e.g., U.S. Pat. Nos. 6,793,923 and 7,824,686; US Patent Publication No. 20160067335, WO 2002/038175; WO 2007/041832; WO 2009/039628; WO 2009/043165 WO 2009/146523, WO 2013049941, WO 2014/153636, WO 2016/176761, WO 2016/109880, WO 2017/190242, WO 2017/083963, WO 2018/058230, WO2019/010560, WO2019/090411, or WO2021/072535 each of which is incorporated herein by reference in their entirety for all intended purposes). The DepoVax™/DPX™ platform is a T cell activation therapeutic delivery formulation that provides controlled and prolonged exposure of antigens plus adjuvant to the immune system. The platform is capable of providing a strong, specific and sustained immune response and is capable of single-dose effectiveness.

In certain embodiments, the T cell activation therapeutic of the invention comprises at least one survivin antigen, wherein each survivin antigen is at a concentration of about 0.01 mg/ml to about 10 mg/ml, about 0.025 mg/ml to about 9 mg/ml, about 0.05 mg/ml to about 8 mg/ml, about 0.75 mg/ml to about 7 mg/ml, about 0.1 mg/ml to about 6 mg/ml, about 0.25 mg/ml to about 5 mg/ml, about 0.5 mg/ml to about 4 mg/ml, about 0.75 mg/ml to about 3 mg/ml, about 1 mg/ml to about 2 mg/ml. In certain embodiments, the T cell activation therapeutic of the invention comprises at least one survivin antigen, wherein each survivin antigen is at a concentration of about 0.1 mg/ml to about 5 mg/ml, about 0.5 mg/ml to about 3 mg/ml, or about 0.5 mg/ml to about 2 mg/ml. In certain embodiments, the T cell activation therapeutic of the invention comprises at least one survivin antigen, wherein each survivin antigen is at a concentration of about 0.01 mg/ml, about 0.02 mg/ml, about 0.03 mg/ml, about 0.04 mg/ml, about 0.05 mg/ml, about 0.06 mg/ml, about 0.07 mg/ml, about 0.08 mg/ml, about 0.09 mg/ml, about 0.1 mg/ml, about 0.2 mg/ml, about 0.3 mg/ml, about 0.4 mg/ml, about 0.5 mg/ml, about 0.6 mg/ml, about 0.7 mg/ml, about 0.8 mg/ml, about 0.9 mg/ml, about 1 mg/ml, about 2 mg/ml, about 3 mg/ml, about 4 mg/ml, about 5 mg/ml, about 6 mg/ml, about 7 mg/ml, about 8 mg/ml, about 9 mg/ml, about or about 10 mg/ml. In certain embodiments, the T cell activation therapeutic of the invention comprises at least one survivin antigen, wherein each survivin antigen is at a concentration of about 1 mg/ml.

In certain embodiments, the T cell activation therapeutic of the invention comprises at least one survivin antigen, wherein the T cell activation therapeutic is administered at a dose of about 0.01 ml to about 3 ml, about 0.05 ml to about 2 ml, about 0.075 ml to about 1.75 ml, about 0.1 ml to about 1.5 ml, about 0.125 ml to about 1.25 ml, about 0.15 ml to about 1 ml, about 0.175 ml to about 0.75 ml, about 0.2 ml to about 0.5 ml, or about 0.25 ml to about 0.5 ml. In certain embodiments, the T cell activation therapeutic of the invention comprises at least one survivin antigen, wherein the T cell activation therapeutic is administered at a dose of about 0.01 ml to about 1 ml, about 0.5 ml to about 0.75 ml, or about 0.25 ml to about 0.5 ml. In certain embodiments, the T cell activation therapeutic of the invention comprises at least one survivin antigen, wherein the T cell activation therapeutic is administered at a dose of about 0.05 ml, about 0.06 ml, about 0.07 ml, about 0.08 ml, about 0.09 ml, about 0.1 ml, about 0.125 ml, about 0.15 ml, about 0.175 ml, about 0.2 ml, about 0.225 ml, about 0.25 ml, about 0.275 ml, about 0.3 ml, about 0.325 ml, about 0.35 ml, about 0.375 ml, about 0.4 ml, about 0.425 ml, about 0.45 ml, about 0.475 ml, about 0.5 ml, about 0.525 ml, about 0.55 ml, about 0.575 ml, about 0.6 ml, about 0.625 ml, about 0.65 ml, about 0.675 ml, about 0.7 ml, about 0.725 ml, about 0.75 ml, about 0.775 ml, about 0.8 ml, about 0.825 ml, about 0.85 ml, about 0.875 ml, about 0.9 ml, about 0.925 ml, about 0.95 ml, about 0.975 ml, about 1 ml, about 1.25 ml, about 1.5 ml, about 1.75 ml, or about 2 ml. In certain embodiments, the T cell activation therapeutic of the invention comprises at least one survivin antigen, wherein the T cell activation therapeutic is administered at a dose of about 0.25 ml or about 0.5 ml. In certain embodiments, the T cell activation therapeutic of the invention comprises at least one survivin antigen, wherein the T cell activation therapeutic is administered at a dose of about 0.1 ml. In certain embodiments, the dose is a priming dose. In certain embodiments, the dose is a booster dose.

In a further embodiment, the T cell activation therapeutic of the invention is any suitable composition as described above, comprising one or more survivin peptide antigens having the amino acid sequence: FEELTLGEF (SEQ ID NO: 2); FTELTLGEF (SEQ ID NO: 3); LTLGEFLKL (SEQ ID NO: 4); LMLGEFLKL (SEQ ID NO: 5); RISTFKNWPF (SEQ ID NO: 6); RISTFKNWPK (SEQ ID NO: 7); STFKNWPFL (SEQ ID NO: 8); and LPPAWQPFL (SEQ ID NO: 9).

In a further embodiment, the T cell activation therapeutic composition comprises five survivin peptide antigens comprising the amino acid sequences: FTELTLGEF (SEQ ID NO: 3), LMLGEFLKL (SEQ ID NO: 5), RISTFKNWPK (SEQ ID NO: 7), STFKNWPFL (SEQ ID NO: 8), and LPPAWQPFL (SEQ ID NO: 9); a T-helper epitope; an adjuvant; lipid vesicle particles; and a carrier comprising a continuous phase of a hydrophobic substance. The T-helper epitope may, for example, be a peptide comprising the amino acid sequence AQYIKANSKFIGITEL (SEQ ID NO: 10). The adjuvant may, for example, be an RNA or DNA based polynucleotide adjuvant (e.g., polyL:C, poly dIdC, etc.). The lipid vesicle particles may, for example, be comprised of 1,2-Dioleoyl-sn-glycero-3-phosphocholine (DOPC; synthetic phospholipid) and cholesterol. The hydrophobic carrier may, for example, be Montanide® ISA51 VG.

In a particular embodiment, the T cell activation therapeutic of the invention may be an anti-cancer immunotherapeutic DPX-Survivac. DPX-Survivac comprises five synthetic survivin peptide antigens having the amino acid sequences: FTELTLGEF (SEQ ID NO: 3), LMLGEFLKL (SEQ ID NO: 5), RISTFKNWPK (SEQ ID NO: 7), STFKNWPFL (SEQ ID NO: 8), and LPPAWQPFL (SEQ ID NO: 9); a universal T-helper epitope from tetanus toxoid (AQYIKANSKFIGITEL; SEQ ID NO: 10); a polyL:C polynucleotide adjuvant; lipid vesicle particles consisting of DOPC and cholesterol; and the hydrophobic carrier Montanide® ISA 51 VG. Exemplary amounts of each component (per ml of T cell activation therapeutic composition) include, without limitation, 1.0 mg of each survivin antigen; 0.5 mg of T-helper epitope (e.g., SEQ ID NO: 10); 0.4 mg of adjuvant (e.g., polyL:C polynucleotide); 120.0 mg of synthetic DOPC phospholipid; 12.0 mg of cholesterol; and 0.7 ml of hydrophobic carrier (e.g., Montanide® ISA51 VG).

In a particular embodiment, the T cell activation therapeutic of the invention may be an anti-cancer immunotherapeutic DPX-Survivac. DPX-Survivac comprises five synthetic survivin peptide antigens having the amino acid sequences: FTELTLGEF (SEQ ID NO: 3), LMLGEFLKL (SEQ ID NO: 5), RISTFKNWPK (SEQ ID NO: 7), STFKNWPFL (SEQ ID NO: 8), and LPPAWQPFL (SEQ ID NO: 9); a universal T-helper epitope from tetanus toxoid (AQYIKANSKFIGITEL; SEQ ID NO: 10); a dIdC polynucleotide adjuvant; lipid vesicle particles consisting of DOPC and cholesterol; and the hydrophobic carrier Montanide® ISA 51 VG. Exemplary amounts of each component (per ml of T cell activation therapeutic composition) include, without limitation, 1.0 mg of each survivin antigen; 0.5 mg of T-helper epitope (e.g., SEQ ID NO: 10); 0.4 mg of adjuvant (e.g., poly dIdC polynucleotide); 120.0 mg of synthetic DOPC phospholipid; 12.0 mg of cholesterol; and 0.7 ml of hydrophobic carrier (e.g., Montanide® ISA51 VG).

The T cell activation therapeutic may optionally further comprise additional components such as, for example, emulsifiers. A more detailed disclosure of exemplary embodiments of the T cell activation therapeutic, and the components thereof, are described as follows.

(i) Survivin Antigens

The T cell activation therapeutic compositions of the invention comprise at least one survivin antigen. The expression “at least one” is used herein interchangeably with the expression “one or more”. These expressions, unless explicitly stated otherwise herein, refer to the number of different survivin antigens in the T cell activation therapeutic, and not to the quantity of any particular survivin antigen. In accordance with the ordinary meaning of “at least one” or “one or more”, the T cell activation therapeutic composition of the invention contains a minimum of one survivin antigen.

Survivin, also called baculoviral inhibitor of apoptosis repeat-containing 5 (BIRC5), is a protein involved in the negative regulation of apoptosis. It has been classed as a member of the family of inhibitors of apoptosis proteins (IAPs). Survivin is a 16.5 kDa cytoplasmic protein containing a single BIR motif and a highly charged carboxy-terminal coiled region instead of a RING finger. The gene coding for survivin is nearly identical to the sequence of Effector Cell Protease Receptor-1 (EPR-1), but oriented in the opposite direction. The coding sequence for the survivin (Homo sapiens) is 429 nucleotides long (SEQ ID NO: 11) including stop codons. The encoded protein survivin (Homo sapiens) is 142 amino acids long (SEQ ID NO: 1)

Human survivin protein sequence
(SEQ ID NO: 1)
MGAPTLPPAWQPFLKDHRISTFKNWPFLEGCACTPERMAE
AGFIHCPTENEPDLAQCFFCFKELEGWEPDDDPIEEHKKH
SSGCAFLSVKKQFEELTLGEFLKLDRERAKNKIAKETNNK
KKEFEETAKKVRRAIEQLAAMD
Nucleotide sequence encoding human
survivin protein
(SEQ ID NO: 11)
atgggtgccccgacgttgccccctgcctggcagccctttc
tcaaggaccaccgcatctctacattcaagaactggccctt
cttggagggctgcgcctgcaccccggagcggatggccgag
gctggcttcatccactgccccactgagaacgagccagact
tggcccagtgtttcttctgcttcaaggagctggaaggctg
ggagccagatgacgaccccatagaggaacataaaaagcat
tcgtccggttgcgctttcctttctgtcaagaagcagtttg
aagaattaacccttggtgaatttttgaaactggacagaga
aagagccaagaacaaaattgcaaaggaaaccaacaataag
aagaaagaatttgaggaaactgcgaagaaagtgcgccgtg
ccatcgagcagctggctgccatggattga

It is postulated that the survivin protein functions to inhibit caspase activation, thereby leading to negative regulation of apoptosis or programmed cell death. Consistent with this function, survivin has been identified as one of the top genes invariably up-regulated in many types of cancer but not in normal tissue (see e.g., Altieri et al., Lab Invest, 79: 1327-1333, 1999; and U.S. Pat. No. 6,245,523; each of which is incorporated herein by reference in their entirety for all intended purposes). This fact, therefore, makes survivin an ideal target for cancer therapy as cancer cells are targeted while normal cells are not. Indeed, survivin is highly expressed in many tumor types, including a large portion of human cancer, and has reported prognostic value.

T cell activation therapeutics of the invention comprise one or more survivin antigens. As used herein, the term “survivin antigen” encompasses any peptide, polypeptide or variant thereof (e.g., survivin peptide variant) derived from a survivin protein or a fragment thereof. The term “survivin antigen” also encompasses a polynucleotide that encodes a survivin peptide, survivin peptide variant or survivin peptide functional equivalent described herein.

Polynucleotides may be DNA (e.g., genomic DNA or cDNA) or RNA (e.g., mRNA) or combinations thereof. They may be naturally occurring or synthetic (e.g., chemically synthesized). It is contemplated that the polynucleotide may contain modifications of one or more nitrogenous bases, pentose sugars or phosphate groups in the nucleotide chain. Such modifications are well-known in the art and may be for the purpose of e.g., improving stability of the polynucleotide.

In an embodiment, the survivin antigen may comprise the full length survivin polypeptide or a nucleic acid encoding the full length survivin polypeptide. Alternatively, the survivin antigen may be a survivin peptide comprising a fragment of any length of the survivin protein. Exemplary embodiments include a survivin peptide that comprises at least 5, 6, 7, 8, 9, 10, 11, 12, 13, 14, 15, 16, 17, 18, 19, 20, 21, 22, 23, 24, or 25 amino acid residues. In specific embodiments, the survivin peptide consists of a heptapeptide, an octapeptide, a nonapeptide, a decapeptide or an undecapeptide, consisting of 7, 8, 9, 10, 11 consecutive amino acid residues of the survivin protein (e.g., SEQ ID NO: 1), respectively. Particular embodiments of the survivin antigen include survivin peptides of about 9 or 10 amino acids.

Survivin antigens of the invention also encompass variants and functional equivalents of survivin peptides. Variants or functional equivalents of a survivin peptide encompass peptides that exhibit amino acid sequences with differences as compared to the specific sequence of the survivin protein, such as one or more amino acid substitutions, deletions or additions, or any combination thereof. The difference may be measured as a reduction in identity as between the survivin protein sequence and the survivin peptide variant or survivin peptide functional equivalent.

The identity between amino acid sequences may be calculated using algorithms well known in the art. Survivin peptide variants or functional equivalents are to be considered as falling within the meaning of a “survivin antigen” of the invention when they are, preferably, over their entire length, at least 50% identical to a peptide sequence of a survivin protein, such as at least 60% identical, at least 70% identical, at least 75% identical, at least 80% identical, at least 85% identical, at least 90% identical, or at least 95% identical, including 96%, 97%, 98% or 99% identical with a peptide sequence of a survivin protein. In a particular embodiment, the survivin peptide variant has a sequence that is at least 85%, 90%, 95%, 96%, 97%, 98% or 99% identical to a consecutive amino acid sequence of SEQ ID NO: 1.

The survivin protein from which the survivin antigen can be derived is a survivin protein from any animal species in which the protein is expressed. A particular embodiment is the survivin protein from humans (SEQ ID NO: 1). Based on the sequence of the selected survivin protein, the survivin antigen may be derived by any appropriate chemical or enzymatic treatment of the survivin protein or coding nucleic acid. Alternatively, the survivin antigen may be synthesized by any conventional peptide or nucleic acid synthesis procedure with which the person of ordinary skill in the art is familiar.

The survivin antigen of the invention (peptide or nucleic acid) may have a sequence which is a native sequence of survivin. Alternatively, the survivin antigen may be a peptide or nucleic acid sequence modified by one or more substitutions, deletions or additions, such as e.g., the survivin peptide variants or functional equivalents described herein. Exemplary procedures and modifications of survivin peptides that increase the immunogenicity of the peptides include, for example, those described in WO 2004/067023 (incorporated herein by reference in its entirety for all intended purposes) involving amino acid substitutions introduced at anchor positions which increase peptide binding to the HLA class I molecule.

In an embodiment, the survivin antigen is any peptide derived from the survivin protein, or any survivin peptide variant thereof, that is capable of binding MHC Class I HLA molecules. Along these lines, the survivin antigen may be any survivin peptide, or survivin peptide variant thereof, that is capable of inducing or potentiating an immune response in a subject.

In an embodiment, the survivin antigen is a peptide antigen comprising an amino acid sequence from the survivin protein (SEQ ID NO: 1) that is capable of eliciting a cytotoxic T-lymphocyte (CTL) response in a subject, or a nucleic acid molecule encoding said peptide.

In an embodiment, the T cell activation therapeutic comprises one or more synthetic survivin peptides, or variants thereof, based on the amino acid sequence of the survivin protein, such as the amino acid sequence set forth in SEQ ID NO: 1.

Survivin peptides, survivin peptide variants and survivin functional equivalents, and their use for diagnostic and therapeutic purposes, specifically in cancer, have been described, for example, in WO 2004/067023 and WO 2006/081826, each of which is incorporated herein in their entirety for all intended purposes. The novel peptides disclosed in these publications were found to be capable of eliciting cytotoxic T-lymphocyte (CTL) responses in cancer patients. In particular, in WO 2004/067023, it was found that MHC Class I restricted peptides can be derived from the survivin protein, which are capable of binding to MHC Class I HLA molecules and thereby eliciting both ex vivo and in situ CTL immune responses in patients suffering from a wide range of cancer diseases.

In an embodiment, the T cell activation therapeutic of the invention may include any one or more of the survivin peptides, survivin peptide variants or survivin peptide functional equivalents disclosed in WO 2004/067023 and WO 2006/081826.

In another embodiment, the T cell activation therapeutic of the invention may include one or more of a survivin peptide, survivin peptide variant or survivin peptide functional equivalent having the ability to bind any of the MHC Class I molecules selected from HLA-A, HLA-B or HLA-C molecules.

Exemplary MHC Class I HLA-A molecules to which the survivin peptide, survivin peptide variant, or survivin peptide functional equivalent may bind include, without limitation, HLA-A 1, HLA-A2, HLA-A3, HLA-A9, HLA-A10, HLA-A11, HLA-A19, HLA-A23, HLA-A24, HLA-A25, HLA-A26, HLA-A28, HLA-A29, HLA-A30, HLA-A31, HLA-A32, HLA-A33, HLA-A34, HLA-A36, HLA-A43, HLA-A66, HLA-A68, and HLA-A69.

Exemplary MHC Class I HLA-B molecules to which the survivin peptide, survivin peptide variant, or survivin peptide functional equivalent may bind include, without limitation, HLA-B5, HLA-B7, HLA-B8, HLA-B12, HLA-B13, HLA-B14, HLA-B15, HLA-B16, HLA-B17, HLA-B18, HLA-B21, HLA-B22, HLA-B27, HLA-B35, HLA-B37, HLA-B38, HLA-B39, HLA-B40, HLA-B41, HLA-B42, HLA-B44, HLA-B45, HLA-B46 and HLA-B47.

Exemplary MHC Class I HLA-C molecules to which the survivin peptide, survivin peptide variant, or survivin peptide functional equivalent may bind include, without limitation, HLA-C1, HLA-C2, HLA-C3, HLA-C4, HLA-C5, HLA-C6, HLA-C7 and HLA-C16.

In a particular embodiment, the T cell activation therapeutic of the invention may comprise one or more of the survivin peptide antigens selected from: i) FEELTLGEF (SEQ ID NO: 2) [HLA-A1]; ii) FTELTLGEF (SEQ ID NO: 3) [HLA-A1]; iii) LTLGEFLKL (SEQ ID NO: 4) [HLA-A2]; iv) LMLGEFLKL (SEQ ID NO: 5) [HLA-A2]; v) RISTFKNWPF (SEQ ID NO: 6) [HLA-A3]; vi) RISTFKNWPK (SEQ ID NO: 7) [HLA-A3]; vii) STFKNWPFL (SEQ ID NO: 8) [HLA-A24]; viii) LPPAWQPFL (SEQ ID NO: 9) [HLA-B7] or a nucleic acid molecule encoding the survivin peptide antigen.

The above-listed survivin peptides represent, without limitation, exemplary MHC Class I restricted peptides encompassed by the invention. The specific MHC Class I HLA molecule to which each of the survivin peptides is believed to bind is shown on the right in square brackets. A T cell activation therapeutic of the invention may comprise one or more of these survivin peptides, in any suitable combination.

In a further embodiment, the T cell activation therapeutic of the invention comprises any one or more of the five survivin peptides listed below, in any suitable combination: i) FTELTLGEF (SEQ ID NO: 3) [HLA-A1] ii) LMLGEFLKL (SEQ ID NO: 5) [HLA-A2] iii) RISTFKNWPK (SEQ ID NO: 7) [HLA-A3] iv) STFKNWPFL (SEQ ID NO: 8) [HLA-A24] v) LPPAWQPFL (SEQ ID NO: 9) [HLA-B7].

In a particular embodiment, the T cell activation therapeutic composition of the invention comprises all five of the survivin peptide antigens listed above, as found in an anti-cancer immunotherapeutic T cell activation therapeutic DPX-Survivac or any combination of one or more of the peptide antigens. In a preferred embodiment, the composition will comprise all five of the survivin peptide antigen, candidate anti-cancer immunotherapeutic T cell activation therapeutic DPX-Survivac.

In addition to the at least one survivin antigen, further embodiments of the T cell activation therapeutic of the invention may comprise one or more additional antigen useful in the treatment of cancer or useful in inducing or potentiating an immune response against cancer.

Exemplary embodiments of such additional antigens are described below.

(ii) Additional Antigens

Other antigens that may be useful in the compositions of the invention include, without limitation, antigens that are capable of inducing or potentiating an immune response in a subject that would be beneficial in the treatment of a tumor or cancer, e.g., a cell-mediated or humoral mediated immune response.

Cell-mediated immunity is an immune response that does not involve antibodies but rather involves the activation of macrophages and natural killer cells, the production of antigen-specific cytotoxic T lymphocytes and the release of various cytokines in response to an antigen. Cytotoxic T lymphocytes are a sub-group of T lymphocytes (a type of white blood cell) which are capable of inducing the death of infected somatic or tumor cells; they kill cells that are infected with viruses (or other pathogens) or are otherwise damaged or dysfunctional.

Most cytotoxic T cells express T cell receptors that can recognise a specific peptide antigen bound to Class I MHC molecules. These CTLs also express CD8 (CD8+ T cells), which is attracted to portions of the Class I MHC molecule. This affinity keeps the CTL and the target cell bound closely together during antigen-specific activation.

Cellular immunity protects the body by, for example, activating antigen-specific cytotoxic T-lymphocytes that are able to lyse body cells displaying epitopes of foreign antigen on their surface, such as virus-infected cells, cells with intracellular bacteria, and cancer cells displaying tumor antigens; activating macrophages and natural killer cells, enabling them to destroy intracellular pathogens; and stimulating cells to secrete a variety of cytokines that influence the function of other cells involved in adaptive immune responses and innate immune responses.

Accordingly, in further embodiments, the T cell activation therapeutic compositions of the invention may comprise an additional antigen to the one or more survivin antigens. For example, the additional antigen may be, without limitation, a peptide, a suitable native, non-native, recombinant or denatured protein or polypeptide, or a fragment thereof, or an epitope that is capable of inducing or potentiating a CTL immune response in a subject.

The additional antigen may also be a polynucleotide that encodes the polypeptide that functions as an antigen. Nucleic acid-based vaccination strategies are known, wherein a T cell activation therapeutic composition that contains a polynucleotide is administered to a subject. The antigenic polypeptide encoded by the polynucleotide is expressed in the subject, such that the antigenic polypeptide is ultimately present in the subject, just as if the T cell activation therapeutic composition itself had contained the polypeptide. For the purposes of the present invention, the additional antigen, where the context dictates, encompasses such polynucleotides that encode the polypeptide which functions as the antigen.

The term “polypeptide” encompasses any chain of amino acids, regardless of length (e.g., at least 6, 8, 10, 12, 14, 16, 18, or 20 amino acids) or post-translational modification (e.g., glycosylation or phosphorylation), and includes, for example, natural proteins, synthetic or recombinant polypeptides and peptides, epitopes, hybrid molecules, variants, homologs, analogs, peptoids, peptidomimetics, etc. A variant or derivative therefore includes deletions, including truncations and fragments; insertions and additions, for example conservative substitutions, site-directed mutants and allelic variants; and modifications, including peptoids having one or more non-amino acyl groups (for example, sugar, lipid, etc.) covalently linked to the peptide and post-translational modifications. As used herein, the term “conserved amino acid substitutions” or “conservative substitutions” refers to the substitution of one amino acid for another at a given location in the peptide, where the substitution can be made without substantial loss of the relevant function. In making such changes, substitutions of like amino acid residues can be made on the basis of relative similarity of side-chain substituents, for example, their size, charge, hydrophobicity, hydrophilicity, and the like, and such substitutions may be assayed for their effect on the function of the peptide by routine testing. Specific, non-limiting examples of a conservative substitution include the following examples.

TABLE 1
Conservative Amino Acid Substitutions

| Original Residue | Conservative Substitution |
|---|---|
| Ala | Ser |
| Arg | Lys |
| Asn | Gln, His |
| Asp | Glu |
| Cys | Ser |
| Gln | Asn |
| Glu | Asp |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

Polypeptides or peptides that have substantial identity to a preferred antigen sequence may be used. Two sequences are considered to have substantial identity if, when optimally aligned (with gaps permitted), they share at least approximately 50% sequence identity, or if the sequences share defined functional motifs. In alternative embodiments, optimally aligned sequences may be considered to be substantially identical (i.e., to have substantial identity) if they share at least 60%, 70%, 75%, 80%, 85%, 90%, 95%, 96%, 97%, 98%, 99% identity over a specified region. The term “identity” refers to sequence similarity between two polypeptides molecules. Identity can be determined by comparing each position in the aligned sequences. A degree of identity between amino acid sequences is a function of the number of identical or matching amino acids at positions shared by the sequences, for example, over a specified region. Optimal alignment of sequences for comparisons of identity may be conducted using a variety of algorithms, as are known in the art, including the ClustalW program, available at clustalw.genome.ad.jp, the local homology algorithm of Smith and Waterman, 1981, Adv. Appl. Math 2: 482, the homology alignment algorithm of Needleman and Wunsch, 1970, J. Mol. Biol. 48:443, the search for similarity method of Pearson and Lipman, 1988, Proc. Natl. Acad. Sci. USA 85:2444, and the computerised implementations of these algorithms (such as GAP, BESTFIT, FASTA and TFASTA in the Wisconsin Genetics Software Package, Genetics Computer Group, Madison, WI, U.S.A.). Sequence identity may also be determined using the BLAST algorithm, described in Altschul et al., 1990, J. Mol. Biol. 215:403-10 (using the published default settings). For example, the “BLAST 2 Sequences” tool, available through the National Center for Biotechnology Information (through the internet at ncbi.nlm.nih.gov/BLAST/bl2seq/wblast2.cgi) may be used, selecting the “blastp” program at the following default settings: expect threshold 10; word size 3; matrix BLOSUM 62; gap costs existence 11, extension 1. In another embodiment, the person skilled in the art can readily and properly align any given sequence and deduce sequence identity and/or homology by mere visual inspection.

Polypeptides and peptides used as an additional antigen in the T cell activation therapeutic composition of the invention can be isolated from natural sources, be synthetic, or be recombinantly generated polypeptides. Peptides and proteins can be recombinantly expressed in vitro or in vivo. The peptides and polypeptides used to practice the invention can be made and isolated using any method known in the art. Polypeptide and peptides used to practice the invention can also be synthesized, whole or in part, using chemical methods well known in the art. See e.g., Caruthers (1980) Nucleic Acids Res. Symp. Ser. 215-223; Horn (1980) Nucleic Acids Res. Symp. Ser. 225-232; Banga, A. K, Therapeutic Peptides and Proteins, Formulation,

Processing and Delivery Systems (1995) Technomic Publishing Co., Lancaster, Pa. For example, peptide synthesis can be performed using various solid-phase techniques (see e.g., Roberge (1995) Science 269:202; Merrifield (1997) Methods Enzymol. 289:3-13) and automated synthesis may be achieved, e.g., using the ABI 431A Peptide Synthesizer (Perkin Elmer) in accordance with the instructions provided by the manufacturer.

In some embodiments, the additional antigen may be a purified antigen, e.g., from about 25% to 50% pure, from about 50% to about 75% pure, from about 75% to about 85% pure, from about 85% to about 90% pure, from about 90% to about 95% pure, from about 95% to about 98% pure, from about 98% to about 99% pure, or greater than 99% pure.

As noted above, the additional antigen includes a polynucleotide that encodes the polypeptide that functions as the antigen. As used herein, the term “polynucleotide” encompasses a chain of nucleotides of any length (e.g., 9, 12, 18, 24, 30, 60, 150, 300, 600, 1500 or more nucleotides) or number of strands (e.g., single-stranded or double-stranded). Polynucleotides may be DNA (e.g., genomic DNA or cDNA) or RNA (e.g., mRNA) or combinations thereof. They may be naturally occurring or synthetic (e.g., chemically synthesized). It is contemplated that the polynucleotide may contain modifications of one or more nitrogenous bases, pentose sugars or phosphate groups in the nucleotide chain. Such modifications are well-known in the art and may be for the purpose of e.g., improving stability of the polynucleotide.

The polynucleotide may be delivered in various forms. In some embodiments, a naked polynucleotide may be used, either in linear form, or inserted into a plasmid, such as an expression plasmid. In other embodiments, a live vector such as a viral or bacterial vector may be used.

One or more regulatory sequences that aid in transcription of DNA into RNA and/or translation of RNA into a polypeptide may be present. In some instances, such as in the case of a polynucleotide that is a messenger RNA (mRNA) molecule, regulatory sequences relating to the transcription process (e.g., a promoter) are not required, and protein expression may be affected in the absence of a promoter. The skilled artisan can include suitable regulatory sequences as the circumstances require.

In some embodiments, the polynucleotide is present in an expression cassette, in which it is operably linked to regulatory sequences that will permit the polynucleotide to be expressed in the subject to which the composition of the invention is administered. The choice of expression cassette depends on the subject to which the composition is administered as well as the features desired for the expressed polypeptide.

Typically, an expression cassette includes a promoter that is functional in the subject and can be constitutive or inducible; a ribosome binding site; a start codon (ATG) if necessary; the polynucleotide encoding the polypeptide of interest; a stop codon; and optionally a 3′ terminal region (translation and/or transcription terminator). Additional sequences such as a region encoding a signal peptide may be included. The polynucleotide encoding the polypeptide of interest may be homologous or heterologous to any of the other regulatory sequences in the expression cassette. Sequences to be expressed together with the polypeptide of interest, such as a signal peptide encoding region, are typically located adjacent to the polynucleotide encoding the protein to be expressed and placed in proper reading frame. The open reading frame constituted by the polynucleotide encoding the protein to be expressed solely or together with any other sequence to be expressed (e.g., the signal peptide), is placed under the control of the promoter so that transcription and translation occur in the subject to which the composition is administered.

The amount of an additional antigen used in a single treatment with a T cell activation therapeutic composition as described herein may vary depending on the type of antigen and the size of the subject. One skilled in the art will be able to determine, without undue experimentation, the effective amount of an additional antigen to use in a particular application.

In some embodiments, the additional antigen may be at least one CTL epitope capable of inducing a CTL response. For example, the additional antigen may be a CTL epitope derived from a protein identified as being up-regulated in cancer cells.

In an embodiment, the CTL epitope may be an epitope of a tumor-associated protein, such as for example, a melanoma-associated protein. In some embodiments, the melanoma-associated protein is a tyrosine related protein-2 (TRP-2) or p53, which can be obtained by various methods including recombinant technology or chemical synthesis.

The following genes, without limitation, code for tumor-associated proteins that have peptide sequences that can be incorporated as an additional antigens in the T cell activation therapeutic composition of the invention: p53, HPV E6 and E7, ART-4, CAMEL, CEA, Cyp-B, HER2/neu, hTERT, hTRT, iCE, MUC1, MUC2, PRAME, P15, RUI, RU2, SART-1, SART-3, WT1, PSA, tyrosinase, TRP-1, TRP-2, gp100, MART-1/Melan A, MAGE-A1.MAGE-A2, MAGE-A3, MAGE-A6, MAGE-A10, MAGE-A12, BAGE, DAM-6, DAM-10, GAGE-1, GAGE-2, GAGE-3, GAGE-4, GAGE-5, GAGE-6, GAGE-7B, GAGE-8, NA88-A, NY-ESO-1, NY-ESO-1 a (CAG-3), AFP, β-catenin/m, Caspase-8/m, CDK-4/m, ELF2M, GnT-V, G250, Ras, HSP70-2M, HST-2, KIAA0205, MUM-1, MUM-2, MUM-3, Myosin/m, RAGE, SART-2, survivin, TRP-2/INT2, and 707-AP.

In an embodiment, the T cell activation therapeutic composition may comprise a mixture of CTL epitopes associated with cancer as antigens for inducing a CTL response. For example, the antigen may comprise at least one or more of a survivin antigen as described herein, such as for example and without limitation, survivin peptide antigens having the following amino acid sequences: FEELTLGEF (SEQ ID NO: 2); FTELTLGEF (SEQ ID NO: 3); LTLGEFLKL (SEQ ID NO: 4); LMLGEFLKL (SEQ ID NO: 5); RISTFKNWPF (SEQ ID NO: 6); RISTFKNWPK (SEQ ID NO: 7); STFKNWPFL (SEQ ID NO: 8); and LPPAWQPFL (SEQ ID NO: 9), together with at least one additional antigen of a tumor-associated protein.

(iii) T-Helper Epitope

In some embodiments, the T cell activation therapeutic composition of the invention comprises at least one T-helper epitope or T-helper antigen.

T-helper epitopes are a sequence of amino acids (natural or non-natural amino acids) that have T-helper activity. T-helper epitopes are recognised by T-helper lymphocytes, which play an important role in establishing and maximising the capabilities of the immune system and are involved in activating and directing other immune cells, such as for example cytotoxic T lymphocytes.

A T-helper epitope can consist of a continuous or discontinuous epitope. Hence not every amino acid of a T-helper is necessarily part of the epitope. Accordingly, T-helper epitopes, including analogs and segments of T-helper epitopes, are capable of enhancing or stimulating an immune response. Immunodominant T-helper epitopes are broadly reactive in animal and human populations with widely divergent MHC types (Celis et al., (1988) J. Immunol. 140:1808-1815; Demotz et al., (1989) J. Immunol. 142:394-402; Chong et al., (1992) Infect. Immun. 60:4640-4647). The T-helper domain of the subject peptides has from about 10 to about 50 amino acids and preferably from about 10 to about 30 amino acids. When multiple T-helper epitopes are present, then each T-helper epitope acts independently.

In some embodiments, the T-helper epitope may form part of an antigen described herein. In particular, if the antigen is of sufficient size, it may contain an epitope that functions as a T-helper epitope. In other embodiments, the T-helper epitope is a separate molecule from the antigen.

In another embodiment, T-helper epitope analogs may include substitutions, deletions and insertions of from one to about 10 amino acid residues in the T-helper epitope. T-helper segments are contiguous portions of a T-helper epitope that are sufficient to enhance or stimulate an immune response. An example of T-helper segments is a series of overlapping peptides that are derived from a single longer peptide.

In a particular embodiment, the compositions of the invention may comprise as a T-helper epitope or antigen, the modified Tetanus toxin peptide A16L (830 to 844; AQYIKANSKFIGITEL (SEQ ID NO: 10), with an alanine residue added to its amino terminus to enhance stability (Slingluff et al, Clin Cancer Res., 7: 3012-3024, 2001, incorporated herein by reference in its entirety for all intended purposes).

Other sources of T-helper epitopes which may be used in the present compositions include, for example, hepatitis B surface antigen helper T cell epitopes, pertussis toxin helper T cell epitopes, measles virus F protein helper T cell epitope, Chlamydia trachomitis major outer membrane protein helper T cell epitope, diphtheria toxin helper T cell epitopes, Plasmodium falciparum circumsporozoite helper T cell epitopes, Schistosoma mansoni triose phosphate isomerase helper T cell epitopes, Escherichia coli TraT helper T cell epitopes and immune-enhancing analogs and segments of any of these T-helper epitopes.

In some embodiments, the T-helper epitope may be a universal T-helper epitope. A universal T-helper epitope as used herein refers to a peptide or other immunogenic molecule, or a fragment thereof, that binds to a multiplicity of MHC class II molecules in a manner that activates T cell function in a class II (CD4+ T cells)-restricted manner. An example of a universal T-helper epitope is PADRE (pan-DR epitope) comprising the peptide sequence AKXVAAWTLKAAA (SEQ ID NO: 12), wherein X may be cyclohexylalanyl. PADRE specifically has a CD4+T-helper epitope, that is, it stimulates induction of a PADRE-specific CD4+T-helper response.

In addition to the modified tetanus toxin peptide A16L mentioned earlier, Tetanus toxoid has other T-helper epitopes that work in the similar manner as PADRE. Tetanus and diphtheria toxins have universal epitopes for human CD4+ cells (Diethelm-Okita, B. M. et al., J. Infect. Diseases, 181:1001-1009, 2000). In another embodiment, the T-helper epitope may be a tetanus toxoid peptide such as F21 E comprising the peptide sequence FNNFTVSFWLRVPKVS ASHLE (amino acids 947-967; SEQ ID NO: 13).

In certain embodiments, the T-helper epitope is fused to at least one of the one or more survivin antigens in the T cell activation therapeutic composition of the invention or to the additional antigen which may be included in the T cell activation therapeutic composition (e.g., a fusion peptide).

(iv) Adjuvants

In some embodiments, the T cell activation therapeutic composition of the invention comprises one or more pharmaceutically acceptable adjuvants. A large number of adjuvants have been described and are known to those skilled in the art. See, for example, Remington's Pharmaceutical Sciences (Remington's Pharmaceutical Sciences, Mack Publishing Company, Easton, Pa., USA 1985) and The United States Pharmacopoeia: The National Formulary (USP 24 NF19) published in 1999.

Exemplary adjuvants include, without limitation, alum, other compounds of aluminum, Bacillus of Calmette and Guerin (BCG), TiterMax™, Ribi™, Freund's Complete Adjuvant (FCA), CpG-containing oligodeoxynucleotides (CpG ODN), lipopeptides and polynucleotides (e.g., polyL:C, poly dIdC, polyA:U etc.). An exemplary CpG ODN is 5′-TCCATGACGTTCCTGACGTT-3′ (SEQ ID NO: 14). The skilled person can readily select other appropriate CpG ODNs on the basis of the target species and efficacy. An exemplary lipopeptide includes, without limitation, Pam3Cys-SKKK (SEQ ID NO: 16) (EMC Microcollections, Germany) or variants, homologs and analogs thereof. The Pam2 family of lipopeptides has been shown to be an effective alternative to the Pam3 family of lipopeptides.

As used herein, a “polyL:C” or “polyL:C polynucleotide” are polynucleotide molecule (RNA or DNA or a combination of DNA and RNA) containing inosinic acid residues (I) and cytidylic acid residues (C), and which is capable of inducing or enhancing the production of at least one inflammatory cytokine, such as interferon, in a mammalian subject.

PolyI:C polynucleotides can have a length of about 8, 10, 12, 14, 16, 18, 20, 22, 24, 25, 28, 30, 35, 40, 45, 50, 55, 60, 65, 70, 75, 80, 85, 90, 95, 100, 150, 200, 250, 300, 500, 1000 or more residues. The upper limit is not believed to be essential. Preferred polyL:C polynucleotides may have a minimum length of about 6, 8, 10, 12, 14, 16, 18, 20, 22, 24, 26, 28, or 30 nucleotides and a maximum length of about 1000, 500, 300, 200, 100, 90, 80, 70, 60, 50, 45 or 40 nucleotides. In certain embodiments, polyL:C polynucleotides are about 20 or more residues in length (commonly 22, 24, 26, 28 or 30 residues in length). If semi-synthetically made (e.g., using an enzyme), the length of the strand may be 500, 1000 or more residues.

In some embodiments, the polyL:C polynucleotide is double-stranded. In such embodiments, they can be composed of one strand consisting entirely of cytosine-containing nucleotides and one strand consisting entirely of inosine-containing nucleotides, although other configurations are possible. For instance, each strand may contain both cytosine-containing and inosine-containing nucleotides. Non-limiting examples includes those in which each strand contains at least 6 contiguous inosinic or cytidylic acid residues, or 6 contiguous residues selected from inosinic acid and cytidylic acid in any order (e.g., IICIIC, ICICIC or IIICCC). In some instances, either or both strands may additionally contain one or more non-cytosine or non-inosine nucleotides

In other embodiments, the polyL:C polynucleotide may be a single-stranded molecule containing inosinic acid residues (I) and cytidylic acid residues (C). As an example, and without limitation, the single-stranded polyL:C may be a sequence of repeating dIdC. In a particular embodiment, the sequence of the single-stranded polyL:C may be a 26-mer sequence of (IC)13, i.e., ICICICICICICICICICICICICIC (SEQ ID NO: 17). As the skilled person will appreciate, due to their nature (e.g., complementarity), it is anticipated that these single-stranded molecules of repeating dIdC would naturally form homodimers, so they are conceptually similar to polyI/polyC dimers.

In certain embodiments, each strand of a polyL:C polynucleotide may be a homopolymer of inosinic or cytidylic acid residues, or each strand may be a heteropolymer containing both inosinic and cytidylic acid residues. In either case, the polymer may be interrupted by one or more non-inosinic or non-cytidylic acid residues (e.g., uridine), provided there is at least one contiguous region of 6 I, 6 C or 6 I/C residues as described above. Typically, each strand of a polyL:C polynucleotide will contain no more than 1 non-I/C residue per 6 I/C residues, more preferably, no more than 1 non-l/C residue per every 8, 10, 12, 14, 16, 18, 20, 22, 24, 26, 28 or 30 I/C residues.

The inosinic acid or cytidylic acid (or other) residues in the polyL:C polynucleotide may be derivatized or modified as is known in the art, provided the ability of the polyL:C polynucleotide to promote the production of an inflammatory cytokine, such as interferon, is retained. Non-limiting examples of derivatives or modifications include e.g., azido modifications, fluoro modifications, or the use of thioester (or similar) linkages instead of natural phosphodiester linkages to enhance stability in vivo. The polyL:C polynucleotide may also be modified to e.g., enhance its resistance to degradation in vivo by e.g., complexing the molecule with positively charged poly-lysine and carboxymethylcellulose, or with a positively charged synthetic peptide.

In certain embodiments, the T cell activation therapeutic comprises a polyL:C polynucleotide as an adjuvant, such as for example and without limitation, a 26 mer deoxy inosine/cytosine synthetic polynucleotide. In certain embodiments, the T cell activation therapeutic comprises a dIdC DNA polynucleotide as an adjuvant.

The polyL:C polynucleotide will typically be included in the compositions of the invention in an amount from about 0.001 mg to 1 mg per unit dose of the composition. In certain embodiments, the amount of polyL:C polynucleotide will be about 0.04 mg/mL of the T cell activation therapeutic composition.

Other suitable adjuvants of the T cell activation therapeutic are those that activate or increase the activity of TLR2. As used herein, an adjuvant which “activates” or “increases the activity” of a TLR includes any adjuvant, in some embodiments a lipid-based adjuvant, which acts as a TLR agonist. Further, activating or increasing the activity of TLR2 encompasses its activation in any monomeric, homodimeric or heterodimeric form, and particularly includes the activation of TLR2 as a heterodimer with TLR1 or TLR6 (i.e., TLR1/2 or TLR2/6).

An exemplary embodiment of an adjuvant that activates or increases the activity of TLR2 is a lipid-based adjuvant that comprises at least one lipid moiety or lipid component.

As used herein, the expression “lipid moiety” or “lipid component” refers to any fatty acid (e.g., fatty acyls) or derivative thereof, including for example triglycerides, diglycerides, and monoglycerides. Exemplary fatty acids include, without limitation, palmitoyl, myristoyl, stearoyl, and decanoyl groups or any C2 to C30 saturated or unsaturated fatty acyl group, preferably any C14 to C22 saturated or unsaturated fatty acyl group, and more preferably a C16 saturated or unsaturated fatty acyl group. Thus, as referred to herein, the expression “lipid-based adjuvant” encompasses any adjuvant comprising a fatty acyl group or derivative thereof.

Lipid-based adjuvants contain at a minimum at least one lipid moiety, or a synthetic/semi-synthetic lipid moiety analogue, which can be coupled onto an amino acid, an oligopeptide or other molecules (e.g., a carbohydrate, a glycan, a polysaccharide, biotin, Rhodamine, etc.). Thus, without limitation, the lipid-based adjuvant may be, for example, a lipoamino acid, a lipopeptide, a lipoglycan, a lipopolysaccharide or a lipoteichoic acid.

Moreover, a lipid moiety or a structure containing a lipid moiety can be coupled covalently or non-covalently to an antigen to create antigenic compounds with built-in adjuvanting properties. For example, and without limitation, the lipid-based moiety may comprise a cation (e.g., nickel) to provide a positive charge for non-covalent coupling.

In some embodiments, the lipid moiety or lipid component may be naturally occurring, such as for example a cell-wall component (e.g., lipoprotein) from a Gram-positive or Gram-negative bacteria, Rhodopseudomonas viridis, or mycoplasma. In other embodiments, the lipid moiety or lipid component may be synthetic or semi-synthetic.

The lipid-based adjuvant may comprise palmitic acid (PAM) as at least one of the lipid moieties or components of the adjuvant. Such lipid-based adjuvants are referred to herein as a “palmitic acid adjuvant”. Palmitic acid is a low molecular weight lipid found in the immunologically reactive Braun's lipoprotein of Escherichia coli. Other common chemical names for palmitic acid include, for example, hexadecanoic acid in IUPAC nomenclature and 1-Pentadecanecarboxylic acid. The molecular formula of palmitic acid is CH₃(CH₂)₁₄CO₂H. As will be understood to those skilled in the art, it is possible that the lipid chain of palmitic acid may be altered. Exemplary compounds which may be used herein as palmitic acid adjuvants, and methods for their synthesis, are described for example in United States Patent Publications US 2008/0233143; US 2010/0129385; and US 2011/0200632, each of which are incorporated herein in their entirety for all intended purposes.

As described above for lipid moieties generally, a palmitic acid adjuvant contains at a minimum at least one palmitic acid moiety, which can be coupled onto an amino acid, an oligopeptide or other molecules. A palmitic acid moiety or a structure containing palmitic acid can be coupled covalently or non-covalently to an antigen to create antigenic compounds with built-in adjuvanting properties. The palmitic acid moiety or a chemical structure containing palmitic acid can be conjugated to a cysteine peptide (Cys) to allow for various structural configurations of the adjuvant, including linear and branched structures. The cysteine residue has been commonly extended by polar residues such as Serine (Ser) and/or lysine (Lys) at the C terminus to create adjuvant compounds with improved solubility. Palmitic acid containing adjuvant compounds could be admixed with an antigen, associated with antigen through non-covalent interactions, or alternatively covalently linked to an antigen, either directly or with the use of a linker/spacer, to generate enhanced immune responses. Most commonly, two palmitic acid moieties are attached to a glyceryl backbone and a cysteine residue to create dipalmitoyl-S-glyceryl-cysteine (PAM2Cys) or tripalmitoyl-S-glyceryl-cysteine (PAM3Cys), which can also be used in multiple configurations as described above.

Therefore, in an embodiment, the adjuvant of the composition may comprise a palmitic acid moiety or component. The palmitic acid moiety may be modified or manipulated to improve its stability in vitro or in vivo, enhance its binding to receptors (such as for example toll-like receptors as described below) or enhance its biological activity.

In a particular embodiment, the palmitic acid adjuvant may comprise PAM2Cys or PAM3Cys. In another particular embodiment, the palmitic acid adjuvant may be Pam-2-Cys-Ser-(Lys)4 (SEQ ID NO: 18) or Pam-3-Cys-Ser-(Lys)4 (SEQ ID NO: 19). Such palmitic acid adjuvants are available, for example, as research reagents from EMC Microcollections GmbH (Germany) and InvivoGen (San Diego, California, USA). Also available from EMC Microcollections are various analogs of Pam-2-Cys-Ser-(Lys)4 (SEQ ID NO: 18) and Pam-3-Cys-Ser-(Lys)4(SEQ ID NO: 19), including labelled analogs.

The composition of the invention may comprise an adjuvant as described above in combination with at least one other suitable adjuvant. Exemplary embodiments of the at least one other adjuvant encompasses, but is by no means limited to, organic and inorganic compounds, polymers, proteins, peptides, sugars from synthetic, non-biological or biological sources (including but not limited to virosomes, virus-like particles, viruses and bacteria of their components).

Further examples of compatible adjuvants may include, without limitation, chemokines, Toll like receptor agonists, colony stimulating factors, cytokines, 1018 ISS, aluminum salts, Amplivax, AS04, AS15, ABM2, Adjumer, Algammulin, ASO1 B, AS02 (SBASA), ASO2A, BCG, Calcitriol, Chitosan, Cholera toxin, CP-870,893, CpG, polyIC, CyaA, Dimethyldioctadecylammonium bromide (DDA), Dibutyl phthalate (DBP), dSLIM, Gamma inulin, GLA-SE, GM-CSF, GMDP, Glycerol, IC30, IC31, Imiquimod, ImuFact IMP321, IS Patch, ISCOM, ISCOMATRIX, Juvlmmune, LipoVac, LPS, lipid core protein, MF59, monophosphoryl lipid A, Montanide® IMS1312, Montanide® based adjuvants, OK-432, OM-174, OM-197-MP-EC, ONTAK, PepTel vector system, other palmitoyl based molecules, PLG microparticles, resiquimod, squalene, SLR172, YF-17 DBCG, QS21, QuilA, P1005, Poloxamer, Saponin, synthetic polynucleotides, Zymosan, pertussis toxin.

Accordingly, the composition may comprise one or more pharmaceutically acceptable adjuvants. In some embodiments, at least one of the one or more survivin antigens or the additional antigen may be coupled to at least one of the adjuvants.

The amount of adjuvant used depends on the amount of antigen and on the type of adjuvant. One skilled in the art can readily determine the amount of adjuvant needed in a particular application by empirical testing.

(v) Lipids

Any lipid may be used in the composition described herein so long as it is a membrane-forming lipid.

Although any lipid as defined above may be used, particularly suitable lipids may include those with at least one fatty acid chain containing at least 4 carbons, and typically about 4 to 28 carbons. The fatty acid chain may contain any number of saturated and/or unsaturated bonds. The lipid may be a natural lipid or a synthetic lipid. Non-limiting examples of lipids may include phospholipids, sphingolipids, sphingomyelin, cerobrocides, gangliosides, ether lipids, sterols, cardiolipin, cationic lipids and lipids modified with poly (ethylene glycol) and other polymers. Synthetic lipids may without limitation, include the following fatty acid constituents; lauroyl, myristoyl, palmitoyl, stearoyl, arachidoyl, oleoyl, linoleoyl, erucoyl, or combinations of these fatty acids. In some embodiments, the lipid or lipids of the lipid vesicle particle are amphiphilic lipids, meaning that they possess both hydrophilic and hydrophobic (lipophilic) properties.

Lipids suitable for use in the composition of the present disclosure include, but are not limited to phospholipids, cationic lipids, cholesterol and/or cholesterol derivatives, or a combination thereof. It is to be understood that the terms “phospholipids”, “cationic lipids” or “cholesterol derivatives”, are not necessarily mutually exclusive of each other.

Broadly defined, a “phospholipid” is a member of a group of lipid compounds that yield on hydrolysis phosphoric acid, an alcohol, fatty acid, and nitrogenous base. Phospholipids that are preferably used in the preparation of the composition of the present disclosure are those with at least one head group selected from the group consisting of phosphoglycerol, phosphoethanolamine, phosphoserine, phosphocholine and phosphoinositol. More preferred are lipids which are about 94-100% phosphatidylcholine. Such lipids are available commercially in the lecithin Phospholipon® 90 G (Phospholipid GmBH, Germany) or lecithin S100 (Lipoid GmBH, Germany). In some embodiments, the phospholipid used in the preparation of the composition of the present disclosure is dioleoyl phosphatidylcholine (DOPC), 1,2-dipalmitoyl-sn-glycero-3-phosphocholine (DPPC), Dioleoyl Phosphatidylethanolamine (DOPE), 1,2-dipalmitoyl-sn-glycero-3-succinate (DGS), or a combination thereof. In one embodiment, the phospholipid used in the preparation of the composition of the present disclosure is dioleoyl phosphatidylcholine (DOPC). In some embodiments, a mixture of DOPC and unesterified cholesterol may be used. In other embodiments, a mixture of Lipoid S 100 lecithin and unesterified cholesterol may be used.

In one embodiment, the lipid vesicle particles comprise a synthetic lipid. In an embodiment, the lipid vesicle particles comprise synthetic DOPC. In another embodiment, the lipid vesicle particles comprise synthetic DOPC and cholesterol.

In an embodiment, the compositions disclosed herein comprise about 120 mg/ml of DOPC and about 12 mg/ml of cholesterol.

Another common phospholipid is sphingomyelin. Sphingomyelin contains sphingosine, an amino alcohol with a long unsaturated hydrocarbon chain. A fatty acyl side chain is linked to the amino group of sphingosine by an amide bond, to form ceramide. The hydroxyl group of sphingosine is esterified to phosphocholine. Like phosphoglycerides, sphingomyelin is amphipathic.

Lecithin, which also may be used, is a natural mixture of phospholipids typically derived from chicken eggs, sheep's wool, soybean and other vegetable sources.

All of these and other phospholipids may be used in the practice of the invention. Phospholipids can be purchased, for example, from Avanti lipids (Alabastar, AL, USA), Lipoid LLC (Newark, NJ, USA) and Lipoid GmbH (Germany), among various other suppliers.

There are various lipid vesicle particles which may form, and the compositions disclosed herein may comprise a single type of lipid vesicle particle or comprise a mixture of different types of lipid vesicle particles.

The term “lipid vesicle particle” encompasses many different types of structures, including without limitation micelles, reverse micelles, and liposomes (e.g., unilamellar, multilamellar, and multivesicular liposomes).

In some embodiments, the T cell activation therapeutic of the invention comprises lipid vesicle particles (e.g., liposomes). In a particular embodiment, liposomes are included when the T cell activation therapeutic compositions comprise a carrier comprising a continuous phase of a hydrophobic substance as described herein.

Liposomes represent a particular embodiment of an adjuvanting system encompassed by the present invention. In certain embodiments, however, the T cell activation therapeutics of the invention may not include liposomes. For example, in some embodiments of the T cell activation therapeutics, the one or more survivin antigens may be combined with any suitable, active agent, additional therapeutic agent and/or an adjuvant for delivery of the survivin antigen to a subject.

A general discussion of liposomes can be found in Gregoriadis G. Immunol. Today, 11:89-97, 1990; and Frezard, F., Braz. J. Med. Bio. Res., 32:181-189, 1999, each of which are incorporated by reference herein in their entirety for all purposes. As used herein and in the claims, the term “liposomes” is intended to encompass all such vesicular structures as described above, including, without limitation, those described in the art as “niosomes”, “transfersomes” and “virosomes”.

Although any liposomes may be used in this invention, including liposomes made from archaebacterial lipids, particularly useful liposomes use phospholipids and unesterified cholesterol in the liposome formulation. When cholesterol is used, the cholesterol may be used in any amount sufficient to stabilize the lipids in the lipid membrane. In an embodiment, the cholesterol may be used in an amount equivalent to about 10% of the weight of phospholipid (e.g., in a DOPC:cholesterol ratio of 10:1 w/w). The cholesterol may stabilize the formation of phospholipid vesicle particles. If a compound other than cholesterol is used, one skilled in the art can readily determine the amount needed. Other liposome stabilizing compounds are known to those skilled in the art. For example, saturated phospholipids produce liposomes with higher transition temperatures indicating increased stability.

Phospholipids that are preferably used in the preparation of liposomes are those with at least one head group selected from the group consisting of phosphoglycerol, phosphoethanolamine, phosphoserine, phosphocholine (e.g., DOPC; 1,2-Dioleoyl-sn-glycero-3-phosphocholine) and phosphoinositol. More preferred are liposomes that comprise lipids which are 94-100% phosphatidylcholine. Such lipids are available commercially in the lecithin Phospholipon® 90 G. When unesterified cholesterol is also used in liposome formulation, the cholesterol is used in an amount equivalent to about 10% of the weight of phospholipid. If a compound other than cholesterol is used to stabilize the liposomes, one skilled in the art can readily determine the amount needed in the composition. In an embodiment, the phospholipid may be phosphatidylcholine or a mixture of lipids comprising phosphatidylcholine. In an embodiment, the lipid may be DOPC (Lipoid GmbH, Germany) or Lipoid S100 lecithin. In some embodiments, a mixture of DOPC and unesterified cholesterol may be used. In other embodiments, a mixture of Lipoid S100 lecithin and unesterified cholesterol may be used.

In an embodiment, the lipid vesicle particles may form closed vesicular structures. They are typically spherical or substantially spherical in shape, but other shapes and conformations may be formed and are not excluded. By “substantially spherical” it is meant that the lipid vesicle particles are close to spherical, but may not be a perfect sphere. Other shapes of the closed vesicular structures include, without limitation, oval, oblong, square, rectangular, triangular, cuboid, crescent, diamond, cylinder or hemisphere shapes. Any regular or irregular shape may be formed. Exemplary embodiments of closed vesicular structures include, without limitation, single layer vesicular structures (e.g., micelles or reverse micelles) and bilayer vesicular structures (e.g., unilamellar or multilamellar vesicles), or various combinations thereof.

By “single layer” it is meant that the lipids do not form a bilayer, but rather remain in a layer with the hydrophobic part oriented on one side and the hydrophilic part oriented on the opposite side. By “bilayer” it is meant that the lipids form a two-layered sheet, such as with the hydrophobic part of each layer internally oriented toward the center of the bilayer with the hydrophilic part externally oriented. Alternatively, the opposite configuration is also possible, i.e., with the hydrophilic part of each layer internally oriented toward the center of the bilayer with the hydrophobic part externally oriented. The term “multilayer” is meant to encompass any combination of single and bilayer structures. The form adopted may depend upon the specific lipid that is used, and whether the composition is or is not water-free.

The closed vesicular structures may be formed from single layer lipid membranes, bilayer lipid membranes and/or multilayer lipid membranes. The lipid membranes are predominantly comprised of and formed by lipids but may also comprise additional components. For example, and without limitation, the lipid membrane may include stabilizing molecules to aid in maintaining the integrity of the structure. Any available stabilizing molecule may be used.

In an embodiment, the lipid vesicle particle is a bilayer vesicular structure, such as for example, a liposome. Liposomes are completely closed lipid bilayer membranes. Liposomes may be unilamellar vesicles (possessing a single bilayer membrane), multilamellar vesicles (characterized by multimembrane bilayers whereby each bilayer may or may not be separated from the next by an aqueous layer) or multivesicular vesicles (possessing one or more vesicles within a vesicle). In an embodiment, the lipid vesicle particles are liposomes when the compositions herein are not water-free.

In an embodiment, the one or more lipid vesicle particles are comprised of a single layer lipid assembly. There are various types of these lipid vesicle particles which may form, and the compositions disclosed herein may comprise a single type of lipid vesicle particle having a single layer lipid assembly or comprise a mixture of different such lipid vesicle particles.

In an embodiment, the lipid vesicle particles herein have a single layer lipid assembly when the compositions herein are water-free.

In an embodiment, the lipid vesicle particle having a single layer lipid assembly partially or completely surrounds the T cell activation therapeutic. As an example, the lipid vesicle particle may be a closed vesicular structure surrounding the T cell activation therapeutic. In an embodiment, the hydrophobic part of the lipids in the vesicular structure is oriented outwards toward the hydrophobic carrier.

As another example, the one or more lipid vesicle particles having a single layer lipid assembly may comprise aggregates of lipids with the hydrophobic part of the lipids oriented outwards toward the hydrophobic carrier and the hydrophilic part of the lipids aggregating as a core. These structures do not necessarily form a continuous lipid layer membrane. In an embodiment, they are an aggregate of monomeric lipids.

In an embodiment, the one or more lipid vesicle particles having a single layer lipid assembly comprise reverse micelles. A typical micelle in aqueous solution forms an aggregate with the hydrophilic parts in contact with the surrounding aqueous solution, sequestering the hydrophobic parts in the micelle center. In contrast, in a hydrophobic carrier, an inverse/reverse micelle forms with the hydrophobic parts in contact with the surrounding hydrophobic solution, sequestering the hydrophilic parts in the micelle center. A spherical reverse micelle can package an T cell activation therapeutic with hydrophilic affinity within its core (i.e., internal environment).

Without limitation, the size of the lipid vesicle particles having a single layer lipid assembly is in the range of from 2 nm (20 A) to 20 nm (200 A) in diameter. In an embodiment, the size of the lipid vesicle particles having a single layer lipid assembly is between about 2 nm to about 10 nm in diameter. In an embodiment, the size of the lipid vesicle particles having a single layer lipid assembly is about 2 nm, 3 nm, 4 nm, 5 nm, 6 nm, about 7 nm, about 8 nm, about 9 nm, or about 10 nm in diameter. In an embodiment, the maximum diameter of the lipid vesicle particles is about 4 nm or about 6 nm. In an embodiment, the lipid vesicle particles of these sizes are reverse micelles.

In an embodiment, one or more of the T cell activation therapeutics are inside the lipid vesicle particles after solubilization in the hydrophobic carrier. By “inside the lipid vesicle particle” it is meant that the T cell activation therapeutic is substantially surrounded by the lipids such that the hydrophilic components of the T cell activation therapeutic are not exposed to the hydrophobic carrier. In an embodiment, the T cell activation therapeutic inside the lipid vesicle particle is predominantly hydrophilic.

In an embodiment, one or more of the T cell activation therapeutics are outside the lipid vesicle particles after solubilization in the hydrophobic carrier. By “outside the lipid vesicle particle”, it is meant that the T cell activation therapeutic is not sequestered within the environment internal to the lipid membrane or assembly. In an embodiment, the T cell activation therapeutic outside the lipid vesicle particle is predominantly hydrophobic.

(vi) Carriers

In some embodiments, the T cell activation therapeutic of the invention comprises a pharmaceutically acceptable carrier, excipient or diluent. As used herein, a pharmaceutically acceptable carrier refers to any substance suitable for delivering a T cell activation therapeutic composition of the invention, and which is useful in the method of the present invention.

Carriers that can be used with T cell activation therapeutics of the invention are well known in the art, and include, but are by no means limited to, e.g., water, phosphate buffered saline, Ringer's solution, dextrose solution, serum-containing solutions, Hank's solution, other aqueous physiologically balanced solutions, oil-in-water emulsions, oils, water-in-oil emulsions, esters, poly(ethylene-vinyl acetate), copolymers of lactic acid and glycolic acid, poly(lactic acid), gelatin, collagen matrices, polysaccharides, poly(D,L lactide), poly(malic acid), poly(caprolactone), celluloses, albumin, starch, casein, dextran, polyesters, ethanol, mathacrylate, polyurethane, polyethylene, vinyl polymers, glycols, thyroglobulin, albumins such as human serum albumin, tetanus toxoid, polyamino acids such as poly L-lysine, poly L-glutamic acid, influenza, hepatitis B virus core protein, mixtures thereof and the like. See, for example, Remington: The Science and Practice of Pharmacy, 2000, Gennaro, A R ed., Eaton, Pa.: Mack Publishing Co.

In a particular embodiment, the carrier of the T cell activation therapeutic composition is a carrier that comprises a continuous phase of a hydrophobic substance, preferably a liquid hydrophobic substance. The continuous phase may be an essentially pure hydrophobic substance or a mixture of hydrophobic substances. In addition, the carrier may be an emulsion of water in a hydrophobic substance or an emulsion of water in a mixture of hydrophobic substances, provided the hydrophobic substance constitutes the continuous phase. Further, in another embodiment, the carrier may function as an adjuvant.

Hydrophobic substances that are useful in the compositions as described herein are those that are pharmaceutically and/or immunologically acceptable. The carrier is preferably a liquid but certain hydrophobic substances that are not liquids at atmospheric temperature may be liquefied, for example by warming, and are also useful in this invention. In one embodiment, the hydrophobic carrier may be a Phosphate Buffered Saline/Freund's Incomplete Adjuvant (PBS/FIA) emulsion.

Oil or water-in-oil emulsions are particularly suitable carriers for use in the T cell activation therapeutic composition of the invention. Oils should be pharmaceutically and/or immunologically acceptable. Suitable oils include, for example, mineral oils (especially light or low viscosity mineral oil such as Drakeol® 6VR), vegetable oils (e.g., soybean oil), nut oils (e.g., peanut oil), or mixtures thereof. Thus, in a particular embodiment the carrier is a hydrophobic substance such as vegetable oil, nut oil or mineral oil. Animal fats and artificial hydrophobic polymeric materials, particularly those that are liquid at atmospheric temperature or that can be liquefied relatively easily, may also be used.

To enhance immunogenicity of cancer T cell activation therapeutic, an adjuvanting T cell activation therapeutic composition platform designed as been to facilitate a strong and robust immune response to peptide antigens. DepoVax™ (or DPX™) is a water free lipid based, including a TLR-adjuvant and universal T-helper peptide, that can be formulated with any epitope, or mixture of epitopes, to induce a cytotoxic T lymphocyte-mediated immune response (Karkada et al., J Immunother 33(3):2050-261, 2010) and/or a humoral immune response. DPX™ is cleared from the injection site of immunization by phagocytic antigen-presenting cells, which prolongs antigen exposure to the immune system.

It has been shown that a single injection with peptides in DPX™ results in equivalent or better immune responses than multiple vaccinations with peptides in other conventional formulations, such as Montanide ISA51 VG emulsions, similar to VacciMax which was a first-generation emulsion-based T cell activation therapeutic composition platform (Daftarian et al., J Transl Med 5:26, 2007; Mansour et al., J Transl Med 5:20, 2007). A DPX™ based peptide-T cell activation therapeutic composition called DPX-0907 completed a phase I clinical trial in breast, ovarian and prostate cancer patients demonstrating safety and immunogenicity in these advanced patients (Berinstein et al., J Transl Med 10(1): 156, 2012).

Thus, in a particular embodiment, the carrier of the T cell activation therapeutic composition of the invention may be a lipid vesicle particle-based adjuvanting system. Unlike water-in-oil emulsion-based T cell activation therapeutics, which rely on oil entrapping water droplets containing antigen and adjuvant, DepoVax™/DPX™ based formulations rely on lipids and lipid mixture to facilitate the incorporation of antigens and adjuvants directly into the oil, without the need for emulsification. Advantages of this approach include: (1) enhancing the solubility of hydrophilic antigens/adjuvant in oil diluents which otherwise would normally have maximum solubility in aqueous based diluents, and (2) the elimination of cumbersome emulsification procedures prior to T cell activation therapeutic composition administration.

In a preferred embodiment, the carrier is mineral oil or is a mannide oleate in mineral oil solution, such as that commercially available as Montanide® ISA 51 (SEPPIC, France).

In certain embodiments, the compositions may be substantially free of water (e.g., “water-free”). It is possible that the hydrophobic carrier of these “water-free” compositions may still contain small quantities of water, provided that the water is present in the non-continuous phase of the carrier. For example, individual components of the composition may have bound water that may not be completely removed by processes such as lyophilization or evaporation and certain hydrophobic carriers may contain small amounts of water dissolved therein. Generally, compositions of the invention that are “water-free” contain, for example, less than about 10%, 9%, 8%, 7%, 6%, 5%, 4%, 3%, 2%, 1%, 0.5%, 0.1%, 0.05% or 0.01% water on a weight/weight basis of the total weight of the carrier component of the composition.

(vii) Methods of Preparing Exemplary T Cell Activation Therapeutic Compositions

The T cell activation therapeutic compositions may be prepared by known methods in the art having regard to the present disclosure. Exemplary embodiments for preparing the compositions disclosed herein are described below without limitation.

In certain embodiments, the T cell activation therapeutic composition of the invention is one that comprises at least one survivin antigen, lipid vesicle particles and a carrier comprising a continuous phase of a hydrophobic substance.

Methods for making lipid vesicle particle, e.g., liposomes, are well known in the art. See e.g., Gregoriadis (1990) and Frezard (1999) both cited previously. Any suitable method for making lipid vesicle particles may be used in the practice of the invention, or lipid vesicle particles may be obtained from a commercial source. lipid vesicle particles are typically prepared by hydrating the lipid vesicle particle components that will form the lipid bilayer (e.g., phospholipids and cholesterol) with an aqueous solution, which may be pure water or a solution of one or more components dissolved in water, e.g., phosphate-buffered saline (PBS), phosphate-free saline, or any other physiologically compatible aqueous solution.

In an embodiment, a lipid vesicle particle component or mixture of lipid vesicle particle components, such as a phospholipid (e.g., Phospholipon® 90G) or DOPC and cholesterol, may be solubilized in an organic solvent, such as a mixture of chloroform and methanol, followed by filtering (e.g., a PTFE 0.2 μm filter) and drying, e.g., by rotary evaporation, to remove the solvents. Hydration of the resulting lipid mixture may be affected by e.g., injecting the lipid mixture into an aqueous solution or sonicating the lipid mixture and an aqueous solution. During formation of lipid vesicle particles, the lipid vesicle particle components form single bilayers (unilamellar) or multiple bilayers (multilamellar) surrounding a volume of the aqueous solution with which the lipid vesicle particle components are hydrated.

In some embodiments, the lipid vesicle particles are then dehydrated, such as by freeze-drying or lyophilization.

In some embodiments, the lipid vesicle particles are combined with an appropriate carrier, such as a carrier comprising a continuous hydrophobic phase. This can be done in a variety of ways.

If the carrier is composed solely of a hydrophobic substance or a mixture of hydrophobic substances (e.g., use of a 100% mineral oil carrier), the lipid vesicle particles may simply be mixed with the hydrophobic substance, or if there are multiple hydrophobic substances, mixed with any one or a combination of them.

If instead the carrier comprising a continuous phase of a hydrophobic substance contains a discontinuous aqueous phase, the carrier will typically take the form of an emulsion of the aqueous phase in the hydrophobic phase, such as a water-in-oil emulsion. Such compositions may contain an emulsifier to stabilize the emulsion and to promote an even distribution of the lipid vesicle particles. In this regard, emulsifiers may be useful even if a water-free carrier is used, for the purpose of promoting an even distribution of the lipid vesicle particles in the carrier. Typical emulsifiers include mannide oleate (Arlacel™ A), lecithin (e.g., S100 lecithin), a phospholipid, Tween™ 80, and Spans™ 20, 80, 83 and 85. Typically, the volume ratio (v/v) of hydrophobic substance to emulsifier is in the range of about 5:1 to about 15:1 with a ratio of about 10:1 being preferred.

In some embodiments, the lipid vesicle particles may be added to the finished emulsion, or they may be present in either the aqueous phase or the hydrophobic phase prior to emulsification.

The survivin antigen(s) or an additional antigen as described herein may be introduced at various different stages of the formulation process. More than one type of antigen may be incorporated into the composition. As used in this section, the term “antigen” is used generally and can refer to a survivin antigen as described herein, one or more survivin antigens, an additional antigen as described herein or one or more additional antigens, or any combination thereof. The term is used generally to describe how any antigen may be formulated in the T cell activation therapeutic compositions of the invention. The term “antigen” encompasses both the singular form “antigen” and the plural “antigens”. It is not necessary that all antigens be introduced into the T cell activation therapeutic composition in the same way.

In some embodiments, the antigen is present in the aqueous solution used to hydrate the components that are used to form the lipid bilayers of the lipid vesicle particles (e.g., phospholipid(s) and cholesterol). In this case, the antigen will be encapsulated in the lipid vesicle particle, present in its aqueous interior. If the resulting lipid vesicle particles are not washed or dried, such that there is residual aqueous solution present that is ultimately mixed with the carrier comprising a continuous phase of a hydrophobic substance, it is possible that additional antigen may be present outside the lipid vesicle particles in the final product. In a related technique, the antigen may be mixed with the components used to form the lipid bilayers of the lipid vesicle particles, prior to hydration with the aqueous solution. The antigen may also be added to pre-formed lipid vesicle particles, in which case the antigen may be actively loaded into the lipid vesicle particles or bound to the surface of the lipid vesicle particles or the antigen may remain external to the lipid vesicle particles. In such embodiments, prior to the addition of antigen, the pre-formed lipid vesicle particles may be empty lipid vesicle particles (e.g., not containing encapsulated antigen or lipid-based adjuvant) or the pre-formed lipid vesicle particles may contain lipid-based adjuvant incorporated into or associated with the lipid vesicle particles. These steps may preferably occur prior to mixing with the carrier comprising a continuous phase of a hydrophobic substance.

In an alternative approach, the antigen may instead be mixed with the carrier comprising a continuous phase of a hydrophobic substance, before, during, or after the carrier is combined with the lipid vesicle particles. If the carrier is an emulsion, the antigen may be mixed with either or both of the aqueous phase or hydrophobic phase prior to emulsification. Alternatively, the antigen may be mixed with the carrier after emulsification.

The technique of combining the antigen with the carrier may be used together with encapsulation of the antigen in the lipid vesicle particles as described above, such that antigen is present both within the lipid vesicle particles and in the carrier comprising a continuous phase of a hydrophobic substance.

The above-described procedures for introducing the antigen into the composition apply also to the T-helper epitope and/or the adjuvant of the compositions as described herein, in embodiments where they are included. That is, the T-helper epitope and/or adjuvant may be introduced into e.g., one or more of: (1) the aqueous solution used to hydrate the components that are used to form the lipid bilayers of the lipid vesicle particles; (2) the aqueous solution after formation of the lipid bilayers of the lipid vesicle particles; (3) the components used to form the lipid bilayers of the lipid vesicle particles; or (4) the carrier comprising a continuous phase of a hydrophobic substance, before, during, or after the carrier is combined with the lipid vesicle particles. If the carrier is an emulsion, the T-helper epitope and/or adjuvant may be mixed with either or both of the aqueous phase or hydrophobic phase before, during or after emulsification.

The technique of combining the T-helper epitope and/or adjuvant with the carrier may be used together with encapsulation of these components in the lipid vesicle particles, or with addition of these components to the lipid vesicle particles, such that T-helper epitope and/or adjuvant is present inside and/or outside the lipid vesicle particles and in the carrier comprising a continuous phase of a hydrophobic substance.

The T-helper epitope and/or adjuvant can be incorporated in the composition together with the antigen at the same processing step, or separately, at a different processing step. For instance, the antigen, T-helper epitope and adjuvant may all be present in the aqueous solution used to hydrate the lipid bilayer-forming lipid vesicle particle components, such that all three components become encapsulated in the lipid vesicle particles. Alternatively, the antigen and the T-helper epitope may be encapsulated in the lipid vesicle particles, and the adjuvant mixed with the carrier comprising a continuous phase of a hydrophobic substance. In a further embodiment, the T-helper epitope and/or adjuvant may be incorporated into the composition after the antigen encapsulation step by passing the lipid vesicle particle-antigen preparation through a manual mini-extruder and then mixing the obtained lipid vesicle particle-antigen preparation with the lipid-based adjuvant in, for example, phosphate buffer. The T-helper epitope and/or adjuvant may also be incorporated into the composition, either alone or together with antigen, after the lipid vesicle particles have been formed, such that the T-helper epitope and adjuvant may be associated or remain external to the lipid vesicle particles. The T-helper epitope and/or adjuvant may also be incorporated into or associated with lipid vesicle particles prior to addition of antigen, with the antigen remaining outside the pre-formed lipid vesicle particles or loaded into/associated with the lipid vesicle particles by further processing. In such embodiments, the resulting preparation may be lyophilized and then reconstituted in the carrier comprising a continuous phase of a hydrophobic substance. It will be appreciated that many such combinations are possible.

If the composition contains one or more further adjuvants, such additional adjuvants can be incorporated in the composition in similar fashion as described above for the adjuvant or by combining several of such methods as may be suitable for the additional adjuvant(s).

Stabilizers such as sugars, anti-oxidants, or preservatives that maintain the biological activity or improve chemical stability to prolong the shelf life of antigen, adjuvant, the lipid vesicle particles or the continuous hydrophobic carrier, may be added to such compositions.

In some embodiments, an antigen/adjuvant mixture may be used, in which case the antigen and adjuvant are incorporated into the composition at the same time. An “antigen/adjuvant mixture” refers to an embodiment in which the antigen and adjuvant are in the same diluent at least prior to incorporation into the composition. The antigen and adjuvant in an antigen/adjuvant mixture may, but need not necessarily be chemically linked, such as by covalent bonding.

In an embodiment for preparing the composition, a lipid preparation is prepared by dissolving lipids, or a lipid-mixture, in a suitable solvent with gently shaking. The T cell activation therapeutic may then be added to the lipid preparation, either directly (e.g., adding dry active agent and/or immunomodulatory agent) or by first preparing a stock of the T cell activation therapeutic dissolved in a suitable solvent. In certain embodiments, the T cell activation therapeutic is added to, or combined with, the lipid preparation with gently shaking. The T cell activation therapeutic preparation is then dried to form a dry cake, and the dry cake is resuspended in a hydrophobic carrier. The step of drying may be performed by various means known in the art, such as by freeze-drying, lyophilization, spray drying, rotary evaporation, evaporation under pressure, etc. Low heat drying that does not compromise the integrity of the components can also be used.

The “suitable solvent” is one that is capable of dissolving the respective component (e.g., lipids, agents, or both), and can be determined by the skilled person.

In respect of the lipids, in an embodiment the suitable solvent is a polar protic solvent such as an alcohol (e.g., tertbutanol, n-butanol, isopropanol, n-propanol, ethanol or methanol), water, acetate buffer, formic acid or chloroform. In an embodiment, the suitable solvent is 40% tertiary-butanol. The skilled person can determine other suitable solvents depending on the lipids to be used.

In a particular embodiment to prepare the compositions, a lipid-mixture containing DOPC and cholesterol in a 10:1 ratio (w:w) (Lipoid GmBH, Germany) can be dissolved in 40% tertiary-butanol by shaking at 300 RPM at room temperature until dissolved. An active agent/immunomodulatory agent stock can be prepared in DMSO and diluted with 40% tertiary-butanol prior to mixing with the dissolved lipid-mixture. T cell activation therapeutic stock can then be added to the dissolved lipid-mixture with shaking at 300 RPM for about 5 minutes. The preparation can then be freeze-dried. The freeze-dried cake can then be reconstituted in Montanide® ISA 51 VG (SEPPIC, France) to obtain a clear solution. Typically, the freeze-dried cake is stored (e.g., at −20° C.) until the time of administration, when the freeze-dried cake is reconstituted in the hydrophobic carrier.

In another embodiment, to prepare the compositions the T cell activation therapeutic is dissolved in sodium phosphate or sodium acetate buffer with S100 lipids and cholesterol (Lipoid, Germany). These components are then lyophilized to form a dry cake. Just prior to injection, the dry cake is resuspended in Montanide® ISA51 VG oil (SEPPIC, France) to prepare a water-free oil-based composition.

In another embodiment, to prepare the compositions the active agent and/or immunomodulatory agent is dissolved in sodium phosphate or sodium acetate buffer with DOPC and cholesterol (Lipoid, Germany). These components are then lyophilized to form a dry cake. Just prior to injection, the dry cake is resuspended in Montanide® ISA51 VG oil (SEPPIC, France) to prepare a water-free oil-based composition.

In another embodiment, to prepare the compositions the dry cake is mixed with lipid/cholesterol nanoparticles (size ≤110 nm) in sodium phosphate or sodium acetate buffer (100 mM, pH 6.0). The lipid may be DOPC. The components are then lyophilized to form a dry cake. Just prior to injection, the dry cake is resuspended in Montanide® ISA51 VG oil (SEPPIC, France) to prepare a water-free oil-based composition.

In some embodiments, it may be appropriate to include an emulsifier in the hydrophobic carrier to assist in stabilizing the components of the dry cake when they are resuspended in the hydrophobic carrier. The emulsifier is provided in an amount sufficient to resuspend the dry mixture of active agent and/or immunomodulatory agent and lipids in the hydrophobic carrier and maintain the active agent and/or immunomodulatory agent and lipids in a dissolved state in the hydrophobic carrier. For example, the emulsifier may be present at about 5% to about 15% weight/weight or weight/volume of the hydrophobic carrier.

Stabilizers such as sugars, anti-oxidants, or preservatives that maintain the biological activity or improve chemical stability to prolong the shelf life of any of the components, may be added to the compositions.

In an embodiment, methods for preparing the compositions herein may include those disclosed in WO 2009/043165, as appropriate in the context of the present disclosure. In such instances, the active agents and/or immunomodulatory agents as described herein would be incorporated into the compositions in similar fashion as described for antigens in WO 2009/043165.

In an embodiment, methods for preparing the compositions herein may include those disclosed in the publications of WO2019090411 and WO2019010560 involving the use of sized lipid vesicle particles. In such instances, the active agents and/or immunomodulatory agents as described herein would be incorporated into the compositions in similar fashion as described for therapeutic agents in the publications of WO2019090411 and WO2019010560, both of which are incorporated herein by reference in their entirety for all intended purposes.

In a particular embodiment, the T cell activation therapeutic of the invention is DPX-Survivac. An exemplary method to prepare DPX-Survivac follows. However, it will be appreciated that alternate embodiments are also encompassed herein, such as those described above where the antigen, adjuvant and T-helper epitope may be introduced at any stage in the formulation of the T cell activation therapeutic, in any order and may ultimately be found inside, outside or both inside and outside the lipid vesicle particles.

In certain embodiments, to prepare DPX-Survivac complex is formed with the five survivin antigens (e.g., SEQ ID Nos: 3, 5, 7, 8 and 9); adjuvant (e.g., polyL:C or poly dIdC polynucleotide) and lipid vesicle particles (DOPC and cholesterol) in an aqueous buffer by a process of mixing and hydrating lipid components in the presence of the survivin antigens and adjuvant, extruded to achieve a particle size that can be sterile filtered, then filled into vials and lyophilized to a dry cake. The dry cake is then re-suspended in the hydrophobic carrier Montanide ISA51 VG before injection. This exemplary method of preparation may be used with any combination of survivin antigens, any suitable adjuvant and any suitable T-helper epitope.

In certain embodiments, to prepare DPX-Survivac, the five survivin antigens (e.g., SEQ ID Nos: 3, 5, 7, 8 and 9) and adjuvant (e.g., polyL:C or poly dIdC polynucleotide) are added to previously sized lipid vesicle particles (e.g., <100 nm, pdi <0.1), sterile filtered and freeze-dried. The dry cake is then re-suspended in the hydrophobic carrier Montanide ISA51 VG before injection. This exemplary method of preparation may be used with any combination of survivin antigens, any suitable adjuvant and any suitable T-helper epitope.

Antibodies, Antibody Mimetics or Functional Equivalents or Fragments

In an embodiment, the inhibitor of PD-1 or PD-L1, active agent and/or additional therapeutic agent is an antibody, a functional equivalent of an antibody or a functional fragment of an antibody.

Broadly, an “antibody” refers to a polypeptide or protein that consists of or comprises antibody domains, which are understood as constant and/or variable domains of the heavy and/or light chains of immunoglobulins, with or without a linker sequence. In an embodiment, polypeptides are understood as antibody domains if they comprise a beta-barrel sequence consisting of at least two beta-strands of an antibody domain structure connected by a loop sequence. Antibody domains may be of native structure or modified by mutagenesis or derivatization, e.g., to modify binding specificity or any other property.

The term “antibody” refers to an intact antibody. In an embodiment, an “antibody” may comprise a complete (i.e., full-length) immunoglobulin molecule, including e.g., polyclonal, monoclonal, chimeric, humanized and/or human versions having full length heavy and/or light chains. The term “antibody” encompasses any and all isotypes and subclasses, including without limitation the major classes of IgA, IgD, IgE, IgG and IgM, and the subclasses IgG1, IgG2, IgG3, IgG4, IgA1 and IgA2. In an embodiment, the antibody is an IgG. The antibody may be one that is naturally occurring or one that is prepared by any means available to the skilled person, such as for example by using animals or hybridomas, and/or by immunoglobulin gene fragment recombinatorial processes. Antibodies are generally described in, for example, Greenfield, 2014).

In an embodiment, the antibody is in an isolated form, meaning that the antibody is substantially free of other antibodies against a different target antigen and/or comprising a different structural arrangement of antibody domains. In an embodiment, the antibody can be an antibody isolated from the serum sample of mammal. In an embodiment, the antibody is in a purified form, such as provided in a preparation comprising only the isolated and purified antibody as the active ingredient. This preparation may be used in the preparation of a composition of the disclosure. In an embodiment, the antibody is an affinity purified antibody.

The antibody may be of any origin, including natural, recombinant and/or synthetic sources. In an embodiment, the antibody may be of animal origin. In an embodiment, the antibody may be of mammalian origin, including without limitation human, murine, rabbit and goat. In an embodiment, the antibody may be a recombinant antibody.

In an embodiment, the antibody may be a monoclonal antibody, a polyclonal antibody, a chimeric antibody, a humanized antibody, a human antibody or a fully human antibody. The meaning applied to these terms and the types of antibodies encompassed therein will be well understood by the skilled person.

Briefly, and without limitation, the term “chimeric antibody” as used herein refers to a recombinant protein that contains the variable domains (including the complementarity determining regions (CDRs)) of an antibody derived from one species, such for example a rodent, while the constant domains of the antibody are derived from a different species, such as a human. For veterinary applications, the constant domains of the chimeric antibody may be derived from that of an animal, such as for example a cat or dog.

Without limitation, a “humanized antibody” as used herein refers to a recombinant protein in which the CDRs from an antibody from one species; e.g., a rodent, are transferred from the heavy and light variable chains of the rodent antibody into human heavy and light variable domains, including human framework region (FR) sequences. The constant domains of the humanized antibody are likewise derived from a human antibody.

Without limitation, a “human antibody” as used herein refers to an antibody obtained from transgenic animals (e.g., mice) that have been genetically engineered to produce specific human antibodies in response to antigenic challenge. In this technique, elements of the human heavy and light chain loci are introduced into strains of mice derived from embryonic stem cell lines that contain targeted disruptions of the endogenous heavy chain and light chain loci. The transgenic animal can synthesize human antibodies specific for human antigens, and the animal can be used to produce human antibody-secreting hybridomas. Methods for obtaining human antibodies from transgenic mice are described e.g., by Green, 1994; Lonberg, 1994; and Taylor, 1994. A fully human antibody also can be constructed by genetic or chromosomal transfection methods, as well as phage display technology, all of which are known in the art. (See, e.g., McCafferty, 1990, for the production of human antibodies and fragments thereof in vitro, from immunoglobulin variable domain gene repertoires from unimmunized donors). In this technique, antibody variable domain genes are cloned in-frame into either a major or minor coat protein gene of a filamentous bacteriophage and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. In this way, the phage mimics some of the properties of the B cell. Phage display can be performed in a variety of formats, for their review, see, e.g., Johnson and Chiswell, 1993. Human antibodies may also be generated by in vitro activated B cells (see, e.g., U.S. Pat. Nos. 5,567,610 and 5,229,275).

As used herein, the term “functional fragment”, with respect to an antibody, refers to an antigen-binding portion of an antibody. In this context, by “functional” it is meant that the fragment maintains its ability to bind to the target antigen. In an embodiment, the binding affinity may be equivalent to, or greater than, that of parent antibody. In an embodiment, the binding affinity may be less than the parent antibody, but nevertheless the functional fragment maintains a specificity and/or selectivity for the target antigen.

In an embodiment, in addition to the functional fragment maintaining its ability to bind to the target antigen of the parent antibody, the functional fragment also maintains the effector function of the antibody, if applicable (e.g., activation of the classical complement pathway; antibody dependent cellular cytotoxicity (ADCC); other downstream signalling processes).

Functional fragments of antibodies include, without limitation, a portion of an antibody such as a F(ab′)₂, a F(ab)₂, a Fab′, a Fab, a Fab₂, a Fab₃, a single domain antibody (e.g., a Dab or VHHs) and the like, including half-molecules of IgG4 (van der Neut Kolfschoten, 2007). Regardless of structure, a functional fragment of an antibody binds with the same antigen that is recognized by the intact antibody. The term “functional fragment”, in relation to antibodies, also includes isolated fragments consisting of the variable regions, such as the “Fv” fragments consisting of the variable regions of the heavy and light chains and recombinant single chain polypeptide molecules in which light and heavy chain variable regions are connected by a peptide linker (“scFv proteins”). As used herein, the term “functional fragment” does not include fragments such as Fc fragments that do not contain antigen-binding sites.

Antibody fragments, such as those described herein, can be incorporated into single domain antibodies (e.g., nanobodies), single-chain antibodies, maxibodies, evibodies, minibodies, intrabodies, diabodies, triabodies, tetrabodies, vNAR, bis-scFv and other like structures (see e.g., Hollinger and Hudson, 2005). Antibody polypeptides including fibronectin polypeptide monobodies, also are disclosed in U.S. Pat. No. 6,703,199. Other antibody polypeptides are disclosed in U.S. Patent Publication No. 20050238646. Each reference cited herein is incorporated by reference in their entirety for all purposes.

Another form of a functional fragment is a peptide comprising one or more CDRs of an antibody or one or more portions of the CDRs, provided the resultant peptide retains the ability to bind the target antigen.

A functional fragment may be a synthetic or genetically engineer protein. For example, functional fragments include isolated fragments consisting of the light chain variable region, “Fv” fragments consisting of the variable regions of the heavy and light chains, and recombinant single chain polypeptide molecules which light and heavy regions are connected by a peptide linker (scFv proteins).

As used herein, the terms “antibody” and “functional fragments” of antibodies encompass any derivatives thereof. By “derivatives” it is meant any modification to the antibody or functional fragment, including both modifications that occur naturally (e.g., in vivo) or that are artificially introduced (e.g., by experimental design). Non-limiting examples of such modifications include, for example, sequence modifications (e.g., amino acid substitutions, insertions or deletions), post-translational modifications (e.g., phosphorylation, N-linked glycosylation, O-linked glycosylation, acetylation, hydroxylation, methylation, ubiquitylation, amidation, etc.), or any other covalent attachment or incorporation otherwise of a heterologous molecule (e.g., a polypeptide, a localization signal, a label, a targeting molecule, etc.). In an embodiment, modification of the antibody or functional fragment thereof may be made to generate a bispecific antibody or fragment (i.e., having more than one antigen-binding specificity) or a bifunctional antibody or fragment (i.e., having more than one effector function).

As used herein, a “functional equivalent” in the context of an antibody refers to a polypeptide or other compound or molecule having similar binding characteristics as an antibody to a particular target, but not necessarily being a recognizable “fragment” of an antibody. In an embodiment, a functional equivalent is a polypeptide having an equilibrium dissociation constant (K_{D}) for a particular target in the range of 10⁻⁷ to 10⁻¹². In an embodiment, the functional equivalent has a K_{D} for a particular target of 10⁻⁸ or lower. In an embodiment, the functional equivalent has a K_{D} for a particular target of 10⁻¹⁰ or lower. In an embodiment, the functional equivalent has a K_{D} for a particular target of 10⁻¹¹ or lower. In an embodiment, the functional equivalent has a K_{D} for a particular target of 10⁻¹² or lower. The equilibrium constant (K_{D}) as defined herein is the ratio of the dissociation rate (K-off) and the association rate (K-on) of a compound to its target.

In an embodiment, the antibody, functional fragment thereof or functional equivalent thereof, is one that binds a target on an immune cell, binds a protein or polypeptide produced by an immune cell, or binds a protein or polypeptide that interacts with or exerts a function upon immune cells (e.g., a ligand).

In an embodiment, the antibody, functional fragment thereof or functional equivalent thereof, is one that has an immunomodulatory activity or function. By “immunomodulatory activity or function”, it is meant that the antibody, functional fragment thereof or functional equivalent thereof can enhance (upregulate), suppress (downregulate), direct, redirect or reprogram the immune response.

In an embodiment, the antibody, functional fragment thereof or functional equivalent thereof, is one that binds to a stimulatory checkpoint molecule and/or an inhibitory checkpoint molecule, such has for example, and without limitation, those described herein. In an embodiment, the antibody, functional fragment thereof or functional equivalent thereof, is an agonist or an antagonist of a stimulatory checkpoint molecule and/or an inhibitory checkpoint molecule. In an embodiment, the antibody, functional fragment thereof or functional equivalent thereof, is an antagonist of an inhibitory checkpoint molecule. In an embodiment, the antibody, functional fragment thereof or functional equivalent thereof, is an agonist or super agonist of a stimulatory checkpoint molecule.

In various embodiments, the inhibitor of PD-1 or PD-L1 is an antibody, a functional fragment thereof or a functional equivalent thereof, or any combination thereof.

PD-1/PD-L1 modulates T cell response. The normal function of PD-1, expressed on the cell surface of activated T cells under healthy conditions, is to down-modulate unwanted or excessive immune responses, including autoimmune reactions. The PD-1 pathway represents a major immune control switch that may be engaged by tumor cells to overcome active T cell immune surveillance, and it is regularly hijacked by tumors to suppress immune control. Tregs that express PD-1 have been shown to have an immune inhibitor response and PD-1/PD-L1 expression is thus thought to play a role in self-tolerance. In the context of cancer, tumor cells over express PD-1 and PD-L1 in order to evade recognition by the immune system. Anti-cancer therapy that blocks the PD-L1/PD-1 increases effector T cell activity and decreases suppressive Treg activity which allows recognition and destruction of the tumor by an individual's immune system.

In some embodiments, the methods described herein involve the use of an anti-PD-1 antibody, a functional fragment thereof or a functional equivalent thereof, or any combination thereof. PD-1 (CD279) is a cell surface receptor that, functioning as an immune checkpoint, downregulates immune responses and promotes self tolerance. In an embodiment, the PD1 antibody can be, but is not limited to, nivolumab (Opdivo™; Bristol-Myers Squibb), pembrolizumab (Keytruda™; Merck), pidilizumab (Cure Tech), AMP-224 (MedImmune & GSK), or RMP1-4 or J43 (BioXCell) or a human or humanized counterpart thereof. In certain embodiments, the PD-1 antibody can be pembrolizumab.

In some embodiments, the methods described herein involve the use of an anti-PD-L1 antibody, a functional fragment thereof or a functional equivalent thereof, or any combination thereof. PD-L1 is a ligand of the PD-1 receptor, and binding to its receptor transmits an inhibitory signal that reduces proliferation of CD8+ T cells and can also induce apoptosis. In an embodiment, the PDL1 antibody can be, but is not limited to, BMS-936559 (Bristol Myers Squibb), atezolizumab (MPDL3280A; Roche), avelumab (Merck & Pfizer), durvalumab (MEDI4736; MedImmune/AstraZeneca), tislelizumab (BeiGene), or cemiplimab (Regeneron).

In other embodiments, and without limitation, the antibody, functional fragment or functional equivalent thereof, may be an anti-PD-1 or anti-PD-L1 antibody, such as for example those disclosed in WO 2015/103602, which is incorporate herein by reference in its entirety for all intended purposes.

In an embodiment, the additional therapeutic agent can be an anti-CTLA-4 antibody, a functional fragment thereof or a functional equivalent thereof, or any combination thereof. CTLA-4 (CD152) is a protein receptor that, functioning as an immune checkpoint, downregulates immune responses. In an embodiment, the antiCTLA4 antibody inhibits CTLA-4 activity or function, thereby enhancing immune responses. In an embodiment, the anti-CTLA-4 antibody can be, but is not limited to, ipilimumab (Bristol-Myers Squibb), tremelimumab (Pfizer; AstraZeneca) or BN-13 (BioXCell). In another embodiment, the antiCTLA-4 antibody can be UC10-4F10-11, 9D9 or 9H10 (BioXCell) or a human or humanized counterpart thereof.

In an embodiment, the active agent is an antibody mimetic, a functional equivalent of an antibody mimetic, or a functional fragment of an antibody mimetic.

As used herein, the term “antibody mimetic” refers to compounds which, like antibodies, can specifically and/or selectively bind antigens or other targets, but which are not structurally related to antibodies. Antibody mimetics are usually artificial peptides or proteins, but they are not limited to such embodiments. Typically, antibody mimetics are smaller than antibodies, with a molar mass of about 3-20 kDa (whereas antibodies are generally about 150 kDa). Non-limiting examples of antibody mimetics include peptide aptamers, affimers, affilins, affibodies, affitins, alphabodies, anticalins, avimers, DARPins™, fynomers, Kunits domain peptides, nanoCLAMPs™, affinity reagents and scaffold proteins. Nucleic acids and small molecules may also be antibody mimetics.

The term “peptide aptamer”, as used herein, refers to peptides or proteins that are designed to interfere with other protein interactions inside cells. They consist of a variable peptide loop attached at both ends to a protein scaffold. This double structural constraint greatly increases the binding affinity of the peptide aptamer to levels comparable to an antibody's (nanomolar range). The variable peptide loop typically comprises 10 to 20 amino acids, and the scaffold may be any protein having good solubility properties. Currently, the bacterial protein Thioredoxin-A is a commonly used scaffold protein, the variable peptide loop being inserted within the redox-active site, which is a -Cys-Gly-Pro-Cys-loop (SEQ ID NO: 15) in the wild protein, the two cysteines lateral chains being able to form a disulfide bridge. Peptide aptamer selection can be made using different systems, but the most widely used is currently the yeast two-hybrid system.

The term “affimer”, as used herein, represents an evolution of peptide aptamers. An affimer is a small, highly stable protein engineered to display peptide loops which provides a high affinity binding surface for a specific target protein or antigen. Affimers can have the same specificity advantage of antibodies, but are smaller, can be chemically synthesized or chemically modified and have the advantage of being free from cell culture contaminants. Affimers are proteins of low molecular weight, typically 12 to 14 kDa, derived from the cysteine protease inhibitor family of cystatins. The affimer scaffold is a stable protein based on the cystatin protein fold. It displays two peptide loops and an N-terminal sequence that can be randomised to bind different target proteins with high affinity and specificity.

The term “affilin”, as used herein, refers to antibody mimetics that are developed by using either gamma-B crystalline or ubiquitin as a scaffold and modifying amino-acids on the surface of these proteins by random mutagenesis. Selection of affilins with the desired target specificity is effected, for example, by phage display or ribosome display techniques. Depending on the scaffold, affilins have a molecular weight of approximately 10 kDa (ubiquitin) or 20 kDa (gamma-B crystalline). As used herein, the term affilin also refers to di- or multimerized forms of affilins (Weidle, 2013).

The term “affibody”, as used herein, refers to a family of antibody mimetics which is derived from the Z-domain of staphylococcal protein A. Structurally, affibody molecules are based on a three-helix bundle domain which can also be incorporated into fusion proteins. In itself, an affibody has a molecular mass of around 6 kDa and is stable at high temperatures and under acidic or alkaline conditions. Target specificity is obtained by randomization of 13 amino acids located in two alpha-helices involved in the binding activity of the parent protein domain (Feldwisch and Tolmachev, 2012, which is incorporated herein in its entirety for all intended purposes). In an embodiment, it is an Affibody™ sourced from Affibody AB, Stockholm, Sweden.

A “affitin” (also known as nanofitin) is an antibody mimetic protein that is derived from the DNA binding protein Sac7d of Sulfolobus acidocaldarius. Affitins usually have a molecular weight of around 7 kDa and are designed to specifically bind a target molecule by randomising the amino acids on the binding surface (Mouratou, 2012). In an embodiment, the affitin is as described in WO 2012/085861, which is incorporated herein in its entirety for all intended purposes.

The term “alphabody”, as used herein, refers to small 10 kDa proteins engineered to bind to a variety of antigens. Alphabodies are developed as scaffolds with a set of amino acid residues that can be modified to bind protein targets, while maintaining correct folding and thermostability. The alphabody scaffold is computationally designed based on coiled-coil structures, but it has no known counterpart in nature. Initially, the scaffold was made of three peptides that associated non-covalently to form a parallel coiled-coil trimer (US Patent Publication No. 20100305304) but was later redesigned as a single peptide chain containing three α-helices connected by linker regions (Desmet, 2014).

The term “anticalin”, as used herein, refers to an engineered protein derived from a lipocalin (Beste, 1999); Gebauer and Skerra, 2009). Anticalins possess an eight-stranded O-barrel which forms a highly conserved core unit among the lipocalins and naturally forms binding sites for ligands by means of four structurally variable loops at the open end. Anticalins, although not homologous to the IgG superfamily, show features that so far have been considered typical for the binding sites of antibodies: (i) high structural plasticity as a consequence of sequence variation and (ii) elevated conformational flexibility, allowing induced fit to targets with differing shape.

The term “avimer” (avidity multimers), as used herein, refers to a class of antibody mimetics which consist of two or more peptide sequences of 30 to 35 amino acids each, which are derived from A-domains of various membrane receptors and which are connected by linker peptides. Binding of target molecules occurs via the A-domain and domains with the desired binding specificity can be selected, for example, by phage display techniques. The binding specificity of the different A-domains contained in an avimer may, but does not have to be identical (Weidle, 2013).

The term “DARPin™”, as used herein, refers to a designed ankyrin repeat domain (166 residues), which provides a rigid interface arising from typically three repeated β-turns. DARPins usually carry three repeats corresponding to an artificial consensus sequence, wherein six positions per repeat are randomised. Consequently, DARPins lack structural flexibility (Gebauer and Skerra, 2009).

The term “Fynomer™”, as used herein, refers to a non-immunoglobulin-derived binding polypeptide derived from the human Fyn SH3 domain. Fyn SH3-derived polypeptides are well-known in the art and have been described, e.g., in Grabulovski, 2007; WO 2008/022759; Bertschinger, 2007; Gebauer and Skerra, 2009; and Schlatter, 2012).

A “Kunitz domain peptide” is derived from the Kunitz domain of a Kunitz-type protease inhibitor such as bovine pancreatic trypsin inhibitor (BPTI), amyloid precursor protein (APP) or tissue factor pathway inhibitor (TFPI). Kunitz domains have a molecular weight of approximately 6 kDA and domains with the required target specificity can be selected by display techniques such as phage display (Weidle, 2013).

The term “monobody” (also referred to as “adnectin”), as used herein, relates to a molecule based on the 10th extracellular domain of human fibronectin III (10Fn3), which adopts an Ig-like β-sandwich fold of 94 residues with 2 to 3 exposed loops, but lacks the central disulphide bridge (Gebauer and Skerra, 2009). Monobodies with the desired target specificity can be genetically engineered by introducing modifications in specific loops of the protein. In an embodiment, the monobody is an ADNECTIN™ (Bristol-Myers Squibb, New York, New York).

The term “nanoCLAMP” (CLostridal Antibody Mimetic Proteins), as used herein, refers to affinity reagents that are 15 kDa proteins having tight, selective and gently reversible binding to target molecules. The nanoCLAMP scaffold is based on an IgG-like, thermostable carbohydrate binding module family 32 (CBM32) from a Clostridium perfringens hyaluronidase (Mu toxin). The shape of nanoCLAMPs approximates a cylinder of approximately 4 nm in length and 2.5 nm in diameter, roughly the same size as a nanobody. nanoCLAMPs to specific targets are generated by varying the amino acid sequences and sometimes the length of three solvent exposed, adjacent loops that connect the beta strands making up the beta-sandwich fold, conferring binding affinity and specificity for the target (Suderman, 2017).

The term “affinity reagent”, as used herein, refers to any compound or substance that binds to a larger target molecule to identify, track, capture or influence its activity. Although antibodies and peptide aptamers are common examples, many different types of affinity reagents are available to the skilled person. In an embodiment, the affinity reagent is one that provides a viable scaffold that can be engineered to specifically bind a target (e.g., Top7 is a scaffold engineered specifically to bind CD4; Boschek, 2009).

The term “scaffold proteins”, as used herein, refers polypeptides or proteins that interact and/or bind with multiple members of a signalling pathway. They are regulators of many key signalling pathways. In such pathways, they regulate signal transduction and help localize pathway components. Herein, they are encompassed by the term “antibody mimetics” for their ability to specifically and/or selectively bind target proteins, much like antibodies. In addition to their binding function and specificity, scaffold proteins may also have enzymatic activity. Exemplary scaffold proteins include, without limitation, kinase suppressor of Ras 1 (KNS), MEK kinase 1 (MEKK1), B cell lymphoma/leukemia 10 (BCL-10), A-kinase-anchoring protein (AKAP), Neuroblast differentiation-associated protein AHNAK, HOMER1, pellino proteins, NLRP family, discs large homolog 1 (DLG1) and spinophillin (PPP1R9B).

Other embodiments of antibody mimetics include, without limitation, Z domain of Protein A, Gamma B crystalline, ubiquitin, cystatin, Sac7D from Sulfolobus acidocaldarius, lipocalin, A domain of a membrane receptor, ankyrin repeat motive, SH3 domain of Fyn, Kunits domain of protease inhibitors, the 10^{th} type III domain of fibronectin, 3- or 4-helix bundle proteins, an armadillo repeat domain, a leucine-rich repeat domain, a PDZ domain, a SUMO or SUMO-like domain, an immunoglobulin-like domain, phosphotyrosine-binding domain, pleckstrin homology domain, or src homology 2 domain.

As used herein, the term “functional fragment”, with respect to an antibody mimetic, refers any portion or fragment of an antibody mimetic that maintains the ability to bind to its target molecule. The functional fragment of an antibody mimetic may be, for example, a portion of any of the antibody mimetics as described herein. In an embodiment, the binding affinity may be equivalent to, or greater than, that of parent antibody mimetic. In an embodiment, the binding affinity may be less than the parent antibody mimetic, but nevertheless the functional fragment maintains a specificity and/or selectivity for the target antigen.

In an embodiment, in addition to the functional fragment of an antibody mimetic maintaining its ability to bind to the target molecule of the parent antibody mimetic, the functional fragment also maintains the effector function of the antibody mimetic, if applicable (e.g., downstream signalling).

As used herein, a “functional equivalent” in the context of an antibody mimetic refers to a polypeptide or other compound or molecule having similar binding characteristics to an antibody mimetic, but not necessarily being a recognizable “fragment” of an antibody mimetic. In an embodiment, a functional equivalent is a polypeptide having an equilibrium dissociation constant (K_{D}) for a particular target in the range of 10⁻⁷ to 10⁻¹². In an embodiment, the functional equivalent has a K_{D} for a particular target of 10⁻⁸ or lower. In an embodiment, the functional equivalent has a K_{D} for a particular target of 10⁻¹⁰ or lower. In an embodiment, the functional equivalent has a K_{D} for a particular target of 10⁻¹¹ or lower. In an embodiment, the functional equivalent has a K_{D} for a particular target of 10⁻¹² or lower. The equilibrium constant (K_{D}) as defined herein is the ratio of the dissociation rate (K-off) and the association rate (K-on) of a compound to its target.

In an embodiment, the antibody mimetic, functional fragment thereof or functional equivalent thereof, is one that binds a target on an immune cell, binds a protein or polypeptide produced by an immune cell, or binds a protein or polypeptide that interacts with or exerts a function upon immune cells (e.g., a ligand).

In an embodiment, the antibody mimetic, functional fragment thereof or functional equivalent thereof, is one that has an immunomodulatory activity or function. In an embodiment, the antibody mimetic, functional fragment thereof or functional equivalent thereof, is one that binds to a stimulatory checkpoint molecule and/or an inhibitory checkpoint molecule, such has for example, and without limitation, those described herein. In an embodiment, the antibody mimetic, functional fragment thereof or functional equivalent thereof, is an agonist or an antagonist of a stimulatory checkpoint molecule and/or an inhibitory checkpoint molecule. In an embodiment, the antibody mimetic, functional fragment thereof or functional equivalent thereof, is an antagonist of an inhibitory checkpoint molecule (e.g., PD-1 or PDL1). In an embodiment, the antibody mimetic, functional fragment thereof or functional equivalent thereof, is an agonist or super agonist of a stimulatory checkpoint molecule.

The amount of any specific active agent as described herein may depend on the type of agent (e.g., small molecule drug, antibody, functional fragment, etc.). One skilled in the art can readily determine the amount of active agent needed in a particular application by empirical testing.

Small Molecule Drugs

In certain embodiments, the inhibitor of PD-1 or PD-L1, active agent and/or additional therapeutic agent is a small molecule drug. The term “small molecule drug” refers an organic or inorganic compound that may be used to treat, cure, prevent or diagnose a disease, disorder, or condition.

As used herein, the term “small molecule” refers to a low molecular weight compound which may be synthetically produced or obtained from natural sources and has a molecular weight of less than 2000 Daltons (Da), less than 1500 Da, less than 1000 Da, less than 900 Da, less than 800 Da, less than 700 Da, less than 600 Da or less than 500 Da. In an embodiment, the small molecule drug has a molecular weight of about 900 Da or less than 900 Da. More particularly, in an embodiment, the small molecule drug has a molecular weight of less than 600 Da, and even more particularly less than 500 Da.

In an embodiment, the small molecule drug has a molecular weight of between about 100 Da to about 2000 Da; about 100 Da to about 1500 Da; about 100 Da to about 1000 Da; about 100 Da to about 900 Da; about 100 Da to about 800 Da; about 100 Da to about 700 Da; about 100 Da to about 600 Da; or about 100 Da to about 500 Da. In an embodiment, the small molecule drug has a molecular weight of about 100 Da, about 150 Da, about 200 Da, about 250 Da, about 300 Da, about 350 Da, about 400 Da, about 450 Da, about 500 Da, about 550 Da, about 600 Da, about 650 Da, about 700 Da, about 750 Da, about 800 Da, about 850 Da, about 900 Da, about 950 Da or about 1000 Da. In an embodiment, the small molecule drug may have a size on the order of 1 nm.

In an embodiment, the small molecule drug is a chemically manufactured active substance or compound (i.e., it is not produced by a biological process). Generally, these compounds are synthesized in the classical way by chemical reactions between different organic and/or inorganic compounds. As used herein, the term “small molecule drug” does not encompass larger structures, such as polynucleotides, proteins, and polysaccharides, which are made by a biological process.

The small molecule drug may exert its activity in the form in which it is administered, or the small molecule drug may be a prodrug. In this regard, the term “small molecule drug”, as used herein, encompasses both the active form and the prodrug.

The term “prodrug” refers to a compound or substance that, under physiological conditions, is converted into the therapeutically active agent. In an embodiment, a prodrug is a compound or substance that, after administration, is metabolized in the body of a subject into the pharmaceutically active form (e.g., by enzymatic activity in the body of the subject). A common method for making a prodrug is to include selected moieties that are hydrolyzed under physiological conditions to reveal the pharmaceutically active form.

In an embodiment, and without limitation, the small molecule drug is a cytotoxic agent, an anti-cancer agent, an anti-tumor agent, a chemotherapeutic agent, an anti-neoplastic agent, an immunomodulatory agent (e.g., an immune enhancer), an immune response checkpoint inhibitor, an anti-angiogenic, an anti-osteoclastogenic, an enzyme modulator, a biological response modifier, a prodrug, a cytokine, a chemokine, a vitamin, a steroid, a ligand, a targeting agent, a radiopharmaceutical, or a radioisotope.

The small molecule drug as used herein, may be a pharmaceutically acceptable salt thereof. As used herein, the term “pharmaceutically acceptable salt(s)” refers to any salt form of an active agent and/or immunomodulatory agent described herein that are safe and effective for administration to a subject of interest, and that possess the desired biological, pharmaceutical and/or therapeutic activity. Pharmaceutically acceptable salts include salts of acidic or basic groups. Pharmaceutically acceptable acid addition salts may include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Suitable base salts may include, but are not limited to, aluminum, calcium, lithium, magnesium, potassium, sodium, zinc, and diethanolamine salts. A review of pharmaceutically acceptable salts can be found, for example, in Berge, 1977, incorporated herein by reference in its entirety for all intended purposes.

In an embodiment, the active agent is an agent that interferes with DNA replication. As used herein, the expression “interferes with DNA replication” is intended to encompass any action that prevents, inhibits or delays the biological process of copying (i.e., replicating) the DNA of a cell. The skilled person will appreciate that there exist various mechanisms for preventing, inhibiting or delaying DNA replication, such as for example DNA cross-linking, methylation of DNA, base substitution, etc. The present disclosure encompasses the use of any agent that interferes with DNA replication. Exemplary, non-limiting embodiments of such agents that may be used are described, for example, in WO2014/153636 and in WO2017/190242, each of which are incorporated herein in their entirety for all purposes. In an embodiment, the agent that interferes with DNA replication is an alkylating agent, such as for example a nitrogen mustard alkylating agent (e.g., cyclophosphamide, bendamustine, chlorambucil, ifosfamide, mechlorethamine, melphalan), a nitrosoureas alkylating agent (e.g., carmustine, lomustine, streptozocin), an alkyl sulfonate alkylating agent (e.g., busulfan), a Triazine alkylating agent (e.g., dacarbazine, temozolomide), or ethylenimine alkylating agent (e.g., altretamine, thiotepa). In certain embodiments, the agent that interferes with DNA replication is cyclophosphamide.

In an embodiment, the active agent is cyclophosphamide (CPA) or a pharmaceutically acceptable salt thereof. Cyclophosphamide (N,N-bis(2-chloroethyl)-1,3,2-oxazaphosphinan-2-amine 2-oxide). The chemical structure of cyclophosphamide is:

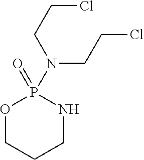 [SMILES: O=P1(N(CCCl)CCCl)NCCCO1]

Cyclophosphamide is also known and referred to under the trademarks Endoxan®, Cytoxan®, Neosar®, Procytox® and Revimmune®. Cyclophosphamide is a prodrug which is converted to its active metabolites, 4-hydroxy-cyclophosphamide and aldophosphamide, by oxidation by P450 enzymes. Intracellular 4-hydroxy-cyclophosphamide spontaneously decomposes into phosphoramide mustard which is the ultimate active metabolite.

The active metabolites of CPA are lipid soluble and enter cells through passive diffusion. Intracellular 4-OH-CPA spontaneously decomposes into phosphoramide mustard which is the ultimate active metabolite. Phosphoramide mustard catalyzes intra- and interstrand DNA cross-links as well as DNA-protein cross-links that inhibit DNA replication leading to cell death (de Jonge, Huitema et al. 2005). Phosphoramide mustard is eliminated by enzymatic conversion to carboxyphoshphamide by cytoplasmic aldehyde dehydrogenase (ALDH) (Emmenegger, Shaked et al., 2007; 2011). Cells with low levels of ALDH tend to accumulate CPA metabolites and are more sensitive to its effects, and indeed tumor upregulation of ALDH is one mechanism of CPA resistance (Zhang, Tian et al. 2005). Besides ALDH, low intracellular ATP levels have also been associated with CPA selectivity towards particular cells types (Zhao, Cao et al. 2010). At high doses, typically in the range of 1-5 g/im2, the effects of CPA are most cytotoxic to rapidly dividing cells indiscriminate of cell type, and CPA is myelosuppressive since most hematogenic cells are rapidly dividing (Bruce, Meeker et al. 1966; Smith and Sladek 1985).

Other nitrogen mustard alkylating agents in the same class as cyclophosphamide include, without limitation, palifosfamide, bendamustine and ifosfamide.

In an embodiment, the active agent and/or additional therapeutic agent can be, but is not limited to, gemcitabine, 5-fluorouracil, cisplatin, oxaliplatin, temozolomide, paclitaxel, thalidomide, capecitabine, methotrexate, epirubicin, idarubicin, mitoxantrone, bleomycin, bortezomib, decitabine, docetaxel, ifosfamide, afosfamide, melphalan, bendamustine, uramustine, palifosfamide, chlorambucil, busulfan, 4-hydroxycyclophosphamide, bis-chloroethylnitrosourea (BCNU), mitomycin C, yondelis, procarbazine, dacarbazine, carboplatin, acyclovir, cytosine arabinoside, ganciclovir, camptothecin, topotecan, irinotecan, doxorubicin, daunorubicin, etoposide, teniposide, or pixantrone, or a pharmaceutically acceptable salt of any one thereof.

In an embodiment, the active agent and/or additional therapeutic agent can be cyclophosphamide, gemcitabine, 5-fluorouracil, cisplatin, oxaliplatin, temozolomide, paclitaxel, thalidomide, capecitabine, methotrexate, epirubicin, idarubicin, mitoxantrone, bleomycin, bortezomib, decitabine, or docetaxel.

In an embodiment, the active agent and/or additional therapeutic agent is one or more of Rituximab, obinutuzumab, Cyclophosphamide, Mitoxantrone, Vincristine, Prednisone, etoposide, Methotrexate/Ifosfamide and Cytarabine, Dexamethasone, Cisplatin, Gemcitabine, Brentuximab vedotin, Bendamustine, liposomal Doxorubicin, Lenalidomide, ibrutinib, selinixor, Polatuzumab vedotin, Pixantrone, CAR-T cells (e.g. Yescarta, Kymriah), mozunetuzumab, bispecific antibodies, thalidomide, autologous stem cell transplant (ASCT), or tafasitamab.

In an embodiment, the small molecule drug can be an immune response checkpoint inhibitor. As used herein, an “immune response checkpoint inhibitor” refers to any compound or molecule that totally or partially modulates (e.g., inhibits or activates) the activity or function of one or more checkpoint molecules (e.g., proteins). Checkpoint molecules are responsible for costimulatory or inhibitory interactions of T cell responses. Checkpoint molecules regulate and maintain self-tolerance and the duration and amplitude of physiological immune responses. Generally, there are two types of checkpoint molecules: stimulatory checkpoint molecules and inhibitory checkpoint molecules.

Stimulatory checkpoint molecules serve a role in enhancing the immune response. Numerous stimulatory checkpoint molecules are known, such as for example and without limitation: CD27, CD28, CD40, CD122, CD137, CD137/4-1BB, ICOS, IL-10, OX40 TGF-beta, TOR receptor, and glucocorticoid-induced TNFR-related protein GITR. In an embodiment, the small molecule drug is an agonist or superagonist of one or more stimulatory checkpoint molecules. The skilled person will be well aware of small molecule drugs that may be used to modulate stimulatory checkpoint molecules.

Inhibitory checkpoint molecules serve a role in reducing or blocking the immune response (e.g., a negative feedback loop). Numerous inhibitory checkpoint proteins are known, such as for example CTLA-4 and its ligands CD80 and CD86; and PD-1 and its ligands PD-L1 and PD-L2. Other inhibitory checkpoint molecules include, without limitation, adenosine A2A receptor (A2AR); B7-H3 (CD276); B7-H4 (VTCN1); BTLA (CD272); killer-cell immunoglobulin-like receptor (KIR); lymphocyte activation gene-3 (LAG3); V-domain Ig suppressor of T cell activation (VISTA); and T cell immunoglobulin domain and mucin domain 3 (TIM-3); as well as their ligands and/or receptors. In an embodiment, the small molecule drug is an antagonist (i.e., an inhibitor) of one or more inhibitory checkpoint molecules. The skilled person will be well aware of small molecule drugs that may be used to modulate inhibitory checkpoint molecules.

In an embodiment, the small molecule drug is an immune response checkpoint inhibitor that is an inhibitor of PD-L1, PD-1, CTLA-4 (CD154), PD-L2 (B7-DC, CD273), LAG3 (CD223), TIM3 (HAVCR2, CD366), 41BB (CD137), 2B4, A2aR, B7H1, B7H3, B7H4, B- and T-lymphocyte attenuator (BTLA), CD2, CD27, CD28, CD30, CD33, CD40, CD70, CD80, CD86, CD160, CD226, CD276, DR3, GAL9, GITR, HVEM, ICOS (inducible T cell costimulator), Killer inhibitory receptor (KIR), LAG-3, LAIR1, LIGHT, MARCO (macrophage receptor with collageneous structure), phosphatidylserine (PS), OX-40, Siglec-5, Siglec-7, Siglec-9, Siglec-11, SLAM, TIGIT, TIM3, TNF-αt, VISTA, VTCN1, or any combination thereof.

In an embodiment, the small molecule drug is an immune response checkpoint agent that is an inhibitor of PD-L1, PD-1, CTLA-4, LAG3, TIM3, 41BB, ICOS, KIR, CD27, OX-40, GITR, or PS, or any combination thereof.

Immunomodulatory Agent

Immunomodulatory agents such as PD-1 or PD-L1 inhibitors can be used in the methods of the present disclosure. The inhibitor of PD-1 or PD-L1 may be any compound, molecule, or substance that inhibits or blocks PD-1 or PD-L1. In certain embodiments, the active agent and/or additional therapeutic agent is an immunomodulatory agent.

As used herein, an “immunomodulatory agent” is a compound or molecule that modulates the activity and/or effectiveness of an immune response. “Modulate”, as used herein, means to enhance (upregulate), direct, redirect or reprogram an immune response. The term “modulate” is not intended to mean activate or induce. By this, it is meant that the immunomodulatory agent modulates (enhances or directs) an immune response that is activated, initiated or induced by a particular substance (e.g., an antigen), but the immunomodulatory agent is not itself the substance against which the immune response is directed, nor is the immunomodulatory agent derived from that substance.

In an embodiment, the immunomodulatory agent is one that modulates myeloid cells (monocytes, macrophages, dendritic cells, magakaryocytes and granulocytes) or lymphoid cells (T cells, B cells and natural killer (NK) cells). In a particular embodiment, the immunomodulatory agent is one that modulates only lymphoid cells. In an embodiment, the immunomodulatory agent is a therapeutic agent that, when administered, stimulates immune cells to proliferate or become activated.

In an embodiment, the immunomodulatory agent is one that enhances the immune response. The immune response may be one that was previously activated or initiated but is of insufficient efficacy to provide an appropriate or desired therapeutic benefit. Alternatively, the immunomodulatory agent may be provided in advance to prime the immune system, thereby enhancing a subsequently activated immune response.

In an embodiment, an immunomodulatory agent that enhances the immune response may be selected from cytokines (e.g., certain interleukins and interferons), stem cell growth factors, lymphotoxins, co-stimulatory molecules, hematopoietic factors, colony stimulating factors, erythropoietins, thrombopoietins, and the like, and synthetic analogs of these molecules.

In an embodiment, an immunomodulatory agent that enhances the immune response may be selected from the following non-limiting examples: lymphotoxins, such as tumor necrosis factor (TNF); hematopoietic factors, such as interleukin (IL); colony stimulating factor, such as granulocyte-colony stimulating factor (G-CSF) or granulocyte macrophage-colony stimulating factor (GM-CSF); interferon, such as interferons-alpha, -beta or -lamda; and stem cell growth factor, such as that designated “SI factor”.

Included among the cytokines are growth hormones, such as, but not limited to, human growth hormone, N-methionyl human growth hormone, and bovine growth hormone; parathyroid hormone; thyroxine; insulin; proinsulin; relaxin; prorelaxin; glycoprotein hormones, such as, but not limited to, follicle stimulating hormone (FSH), thyroid stimulating hormone (TSH), and luteinizing hormone (LH); hepatic growth factor; prostaglandin, fibroblast growth factor; prolactin; placental lactogen, OB protein; tumor necrosis factor-alpha and -beta; mullerian-inhibiting substance; mouse gonadotropin-associated peptide; inhibin; activin; vascular endothelial growth factor (VEGF); integrin; thrombopoietin (TPO); nerve growth factors, such as, but not limited to, NGF-beta; platelet-growth factor; transforming growth factors (TGFs), such as, but not limited to, TGF-alpha and TGFP; insulin-like growth factor-I and —II; erythropoietin (EPO); osteoinductive factors; interferons, such as, but not limited to, interferon-alpha, -beta, and -gamma; colony stimulating factors (CSFs), such as, but not limited to, macrophage-CSF (M-CSF); interleukins (ILs), such as, but not limited to, IL-1, IL-1alpha, IL-2, IL-3, IL-4, IL-5, IL-6, IL-7, IL-8, IL-9, IL-10, IL-11, IL-12; IL-13, IL-14, IL-15, IL-16, IL-17, IL-18, IL-21, IL-25, LIF, kit-ligand or FLT-3, angiostatin, thrombospondin, endostatin and tumor necrosis factor.

In an embodiment, the immunomodulatory agent can be an agent which modulates a checkpoint molecule. Checkpoint molecules are discussed in greater detail above.

In an embodiment, the immunomodulatory agent is any compound, molecule, or substance that is an immune checkpoint inhibitor, including but not limited to, an inhibitor of an immune checkpoint protein selected from Programmed Death-Ligand 1 (PD-L1, also known as B7-H1, CD274), Programmed Death 1 (PD-1, CD279), CTLA-4 (CD154), PD-L2 (B7-DC, CD273), LAG3 (CD223), TIM3 (HAVCR2, CD366), 41BB (CD137), 2B4, A2aR, B7H1, B7H3, B7H4, B- and T-lymphocyte attenuator (BTLA), CD2, CD27, CD28, CD30, CD33, CD40, CD70, CD80, CD86, CD160, CD226, CD276, DR3, GAL9, GITR, HVEM, ICOS (inducible T cell costimulator), Killer inhibitory receptor (KIR), LAG-3, LAIR1, LIGHT, MARCO (macrophage receptor with collageneous structure), phosphatidylserine (PS), OX-40, Siglec-5, Siglec-7, Siglec-9, Siglec-11, SLAM, TIGIT, TIM3, TNF-αt, VISTA, VTCN1, or any combination thereof.

In an embodiment, the immunomodulatory agent is any compound, molecule, or substance that inhibits or blocks CTLA-4. CTLA-4 signaling inhibits T cell activation, particularly during strong T cell responses. CTLA-4 blockade using CTLA-4 inhibitors, such as anti-CTLA-4 monoclonal antibodies, has great appeal because suppression of inhibitory signals results in the generation of an antitumor T cell response. Both clinical and preclinical data indicate that CTLA-4 blockade results in direct activation of CD4+ and CD8+ effector cells, and anti-CTLA-4 monoclonal antibody therapy has shown promise in a number of cancers.

In an embodiment, the immunomodulatory agent may be an immune costimulatory molecule agonist. Immune costimulatory molecules are signaling proteins that play a role in regulating immune response. Some immune costimulatory molecules are receptors located on the surface of a cell that respond to extracellular signaling. When activated, immune costimulatory molecules produce a pro-inflammatory response that can include suppression of regulatory T cells and activation of cytotoxic or killer T cells. Accordingly, immune costimulatory molecule agonists can be used to activate the immune system in an individual to kill cancer cells.

Exemplary immune costimulatory molecules include any of CD27, CD28, CD40, CD122, CD137, CD137/4-1BB, ICOS, IL-10, OX40 TGF-beta, TOR receptor, and glucocorticoid-induced TNFR-related protein GITR. For example, OX40 stimulation suppresses Treg cell function while enhancing effector T cell survival and activity, thereby increasing anti-tumor immunity.

In an embodiment, the immunomodulatory agent is any compound, molecule or substance that is an agonist of a costimulatory immune molecule, including, but not limited to, a costimulatory immune molecule selected from CD27, CD28, CD40, CD122, CD137, CD137/4-1BB, ICOS, IL-10, OX40 TGF-beta, TOR receptor, and glucocorticoid-induced TNFR-related protein GITR.

Various checkpoint inhibitors may be used. For example, the checkpoint inhibitor may be an antibody that binds to and antagonizes an inhibitory checkpoint protein. Exemplary antibodies include anti-PD-1 antibodies (pembrolizumab, nivolumab, pidilizumab, AMP-224, tislelizumab, cemiplimab, RMP1-4 or J43), anti-PD-L1 antibodies (atezolizumab, avelumab, BMS-936559 or durvalumab), anti-CTLA-4 antibodies (ipilimumab, tremelimumab, BN-13, UC10-4F10-11, 9D9 or 9H10) and the like. In some embodiments, the checkpoint inhibitor may be a small molecule or an RNAi that targets an inhibitory checkpoint protein. In some embodiments, the checkpoint inhibitor may be a peptidomimetic or a polypeptide.

Various immune costimulatory molecule agonists may be used. For example, the immune costimulatory molecule agonist may be an antibody that binds to and activates an immune costimulatory molecule. In further embodiments, the immune costimulatory molecule agonist may be a small molecule that targets and activates an immune costimulatory molecule.

In an embodiment, the immunomodulatory agent can be any compound, molecule or substance that is an immunosuppressive cytotoxic drug. In an embodiment, the immunosuppressive cytotoxic drug is a glucocorticoid, a cytostatic (e.g., alkylating agents, antimetabolites), an antibody, a drug acting on immunophilins, an interferon, an opioid, or a TNF binding protein. Immunosuppressive cytotoxic drugs include, without limitation, nitrogen mustards (e.g., cyclophosphamide), nitrosoureas, platinum compounds, folic acid analogs (e.g., methotrexate), purine analogs (e.g., azathioprine and mercaptopurine), pyrimidine analogs (e.g., fluorouracil), protein synthesis inhibitors, cytotoxic antibiotics (e.g., dactinomycin, anthracyclines, mitomycin C, bleomycin and mithramycin), cyclosporine, tacrolimus, sirolimus/rapamycin, everolimus, prednisone, dexamethasone, hydrocortisone, mechlorethamine, clorambucil, mycopholic acid, fingolimod, myriocin, infliximab, etanercept, or adalimumab.

In an embodiment, the immunomodulatory agent can be an anti-inflammatory agent. In one embodiment, the anti-inflammatory agent can be a non-steroidal anti-inflammatory agent. In an embodiment, the non-steroidal anti-inflammatory agent can be a Cox-1 and/or Cox-2 inhibitor. In an embodiment, anti-inflammatory agent includes, without limitation, aspirin, salsalate, diflunisal, ibuprofen, fenoprofen, flubiprofen, fenamate, ketoprofen, nabumetone, piroxicam, naproxen, diclofenac, indomethacin, sulindac, tolmetin, etodolac, ketorolac, oxaprozin, or celecoxib. In an embodiment, the anti-inflammatory agent can be a steroidal anti-inflammatory agent. In an embodiment, the steroidal anti-inflammatory agent can be a corticosteroid.

In an embodiment, the immunomodulatory agent is any one or more of the active agents as described herein (e.g., a small molecule drug, antibody, antibody mimetic or functional equivalent or fragment thereof), whereby the active agent has an immunomodulatory function.

In an embodiment, the immunomodulatory agent is the additional therapeutic agent as described herein (e.g., a small molecule drug, antibody, antibody mimetic or functional equivalent or fragment thereof), whereby the active agent has an immunomodulatory function. In certain embodiments, the additional therapeutic agent is any one or more of epacadostat, rapamycin, doxorubicin, valproic acid, mitoxantrone, vorinostat, cyclophosphamide, irinotecan, cisplatin, methotrexate, tacrolimus, or an anti-CTLA-4 antibody.

The skilled person will be well aware of other immunomodulatory agents encompassed within the above. Notably, the term “immunomodulatory agent”, as used herein, does not encompass compounds or compositions that function to enhance the immunogenicity of an antigen by prolonging the exposure of the antigen to immune cells (i.e., by a delivery platform, such as Freund's™ complete or incomplete adjuvant, Montanide™ ISA, or other oil-based carriers).

The amount of any specific immunomodulatory agent as described herein may depend on the type of agent (e.g., small molecule drug, antibody, etc.). One skilled in the art can readily determine the amount of immunomodulatory agent needed in a particular application by empirical testing.

### Mode of Administration

The methods disclosed herein comprise administering an inhibitor of PD-L1 or PD-1 along with a T cell activation therapeutic (e.g., survivin therapeutic) to a subject with hematologic malignancy. In certain embodiments, the invention further comprises administering one or more active agent (e.g., one that interferes with DNA replication). In certain embodiments, the invention further comprises administering at least one additional therapeutic agent. In certain embodiments, the inhibitor of PD-L1 or PD-1, active agent and/or additional therapeutic agent are administered with the same regimen. In certain embodiments, the inhibitor of PD-L1 or PD-1, active agent and additional therapeutic agent are administered with different regimens.

As used herein, the terms “combination”, “co-administration”, or “combined administration” or the like are meant to encompass administration of the active agent and the T cell activation therapeutic to a single patient, and are intended to include instances where the agent and T cell activation therapeutic are not necessarily administered by the same route of administration or at the same time. For example, the active agent and the T cell activation therapeutic may be administered separately, sequentially, or using alternating administration.

In various embodiments of the present disclosure, an inhibitor of PD-1 or PD-L1 is administered along with a T cell activation therapeutic. In certain embodiments, administration of the inhibitor of PD-1 or PD-L1 and the T cell activation therapeutic to a single patient and are intended to include instances wherein the agent and T cell activation therapeutic are not necessarily administered by the same route of administration or at the same time. For example, the inhibitor of PD-1 or PD-L1 and the T cell activation therapeutic may be administered separately, sequentially, or using alternating administration.

In certain embodiments, the inhibitor of PD-1 or PD-L1 is administered before, at the same time, or after the administration of the T cell activation therapeutic.

In certain embodiments, the inhibitor of PD-1 or PD-L1 is administered in an amount sufficient to provide an immune-modulating effect.

In certain embodiments, the inhibitor of PD-1 or PD-L1 is administered at a dose of about 10 mg to about 1 g, about 5 mg to about 5 g, about 10 mg to about 4.5 g, about 15 mg to about 4 g, about 20 mg to about 3.5 g, about 25 mg to about 3 g, about 30 mg to about 2.5 g, about 35 mg to about 2 g, about 40 mg to about 1.5 g, about 45 mg to about 1 g, about 50 mg to about 900 mg, about 55 mg to about 850 mg, about 60 mg to about 800 mg, about 200 mg to about 800 mg, about 400 mg to about 1000 mg, about 8000 mg to about 1200 mg, about 1000 mg to about 1500 mg, about 65 mg to about 750 mg, about 70 mg to about 700 mg, about 75 mg to about 650 mg, about 80 mg to about 600 mg, about 85 mg to about 550 mg, about 90 mg to about 500 mg, about 95 mg to about 450 mg, about 100 mg to about 400 mg, about 110 mg to about 350 mg, about 120 mg to about 300 mg, about 130 mg to about 290 mg, about 140 mg to about 280 mg, about 150 mg to about 270 mg, about 160 mg to about 260 mg, about 170 mg to about 250 mg, about 180 mg to about 240 mg, about 190 mg to about 230 mg, or about 200 mg to about 220 mg. In certain embodiments, the inhibitor of PD-1 or PD-L1 is administered at a dose of about 50 mg to about 350 mg, about 100 mg to about 300 mg, about 150 mg to about 250 mg, about 300 mg to about 400 mg, about 400 mg to about 500 mg, about 500 mg to about 800 mg, or about 800 mg to about 1200 mg. In certain embodiments, the inhibitor of PD-1 or PD-L1 is administered at a dose of or at least a dose of about 5 mg, at least about 10 mg, at least about 15 mg, at least about 20 mg, at least about 25 mg, at least about 30 mg, at least about 40 mg, at least about 50 mg, at least about 60 mg, at least about 70 mg, at least about 75 mg, at least about 80 mg, at least about 90 mg, at least about 100 mg, at least about 125 mg, at least about 150 mg, at least about 175 mg, at least about 200 mg, at least about 225 mg, at least about 250 mg, at least about 275 mg, at least about 300 mg, at least about 325 mg, at least about 350 mg, at least about 375 mg, at least about 400 mg, at least about 425 mg, at least about 450 mg, at least about 475 mg, at least about 500 mg, at least about 525 mg, at least about 550 mg, at least about 575 mg, at least about 600 mg, at least about 625 mg, at least about 650 mg, at least about 675 mg, at least about 700 mg, at least about 725 mg, at least about 750 mg, at least about 775 mg, at least about 800 mg, at least about 825 mg, at least about 850 mg, at least about 875 mg, at least about 900 mg, at least about 925 mg, at least about 950 mg, at least about 975 mg, at least about 1 g, at least about 2 g, at least about 3 g, at least about 4 g, or at least about 5 g. In certain embodiments, the inhibitor of PD-1 or PD-L1 is administered at a dose of about 100 mg per dose. In certain embodiments, the inhibitor of PD-1 or PD-L1 is administered at about 200 mg per dose. In certain embodiments, the inhibitor of PD-1 or PD-L1 is administered at a dose of about 200 mg. In certain embodiments, the inhibitor of PD-1 or PD-L1 is administered at a dose of about 400 mg. In certain embodiments, the inhibitor of PD-1 or PD-L1 is administered at a dose of about 480 mg. In certain embodiments, the inhibitor of PD-1 or PD-L1 is administered at a dose of about 1200 mg. In certain embodiments, the inhibitor is an inhibitor of PD-1. In certain embodiments, the inhibitor of PD-1 is an antibody. In certain embodiments, the antibody is pembrolizumab. In certain embodiments, the antibody is nivolumab. In certain embodiments, the inhibitor is an inhibitor of PD-L1. In certain embodiments, the inhibitor of PD-L1 is an antibody. In certain embodiments, the antibody is atezolizumab.

In certain embodiments, the inhibitor of PD-1 or PD-L1 is administered at a dose of about 400 mg or less per dose, about 300 mg or less per dose, about 275 mg or less per dose, about 250 mg or less per dose, about 225 mg or less per dose, about 200 mg or less per dose, about 175 mg or less per dose, about 150 mg or less per dose, about 125 mg per dose, or about 100 mg per dose. In certain embodiments, the inhibitor is an inhibitor of PD-1. In certain embodiments, the inhibitor of PD-1 is an antibody. In certain embodiments, the antibody is pembrolizumab. In one embodiment, pembrolizumab is administered at about 200 mg per dose and is administered every 3 weeks. In one embodiment, pembrolizumab is administered at about 400 mg per dose and is administered every 3 weeks.

In certain embodiments, the inhibitor of PD-1 or PD-L1 is administered at a dose of less than about 400 mg per dose, less than about 300 mg per dose, less than about 275 mg per dose, less than about 250 mg per dose, less than about 225 mg per dose, less than about 200 mg per dose, less than about 175 mg per dose, less than about 150 mg per dose, less than about 125 mg per dose, or about 100 mg per dose. In certain embodiments, the inhibitor is an inhibitor of PD-1. In certain embodiments, the inhibitor of PD-1 is an antibody. In certain embodiments, the antibody is pembrolizumab. In one embodiment, pembrolizumab is administered at about 200 mg per dose and is administered every 3 weeks. In one embodiment, pembrolizumab is administered at about 400 mg per dose and is administered every 3 weeks.

In certain embodiments, the inhibitor of PD-1 or PD-L1 is administered at a dose of about 480 mg or less per dose, about 400 mg or less per dose, about 300 mg or less per dose, about 275 mg or less per dose, about 250 mg or less per dose, about 225 mg or less per dose, about 200 mg or less per dose, about 175 mg or less per dose, about 150 mg or less per dose, about 125 mg per dose, or about 100 mg per dose. In certain embodiments, the inhibitor is an inhibitor of PD-1. In certain embodiments, the inhibitor of PD-1 is an antibody. In certain embodiments, the antibody is nivolumab. In one embodiment, nivolumab is administered at about 480 mg per dose.

In certain embodiments, the inhibitor of PD-1 or PD-L1 is administered at a dose of less than about 480 mg per dose, less than about 400 mg per dose, less than about 300 mg per dose, less than about 275 mg per dose, less than about 250 mg per dose, less than about 225 mg per dose, less than about 200 mg per dose, less than about 175 mg per dose, less than about 150 mg per dose, less than about 125 mg per dose, or about 100 mg per dose. In certain embodiments, the inhibitor is an inhibitor of PD-1. In certain embodiments, the inhibitor of PD-1 is an antibody. In certain embodiments, the antibody is nivolumab. In one embodiment, nivolumab is administered at about 480 mg per dose.

In certain embodiments, the inhibitor of PD-1 or PD-L1 is administered at a dose of less than about 1200 mg per dose, less than about 1100 mg per dose, less than about 1000 mg per dose, less than about 900 mg per dose, less than about 800 mg per dose, less than about 700 mg per dose, less than about 600 mg per dose, less than about 500 mg per dose, less than about 400 mg per dose, less than about 300 mg per dose, less than about 275 mg per dose, less than about 250 mg per dose, less than about 225 mg per dose, less than about 200 mg per dose, less than about 175 mg per dose, less than about 150 mg per dose, less than about 125 mg per dose, or about 100 mg per dose. In certain embodiments, the inhibitor is an inhibitor of PD-L1. In certain embodiments, the inhibitor of PD-L1 is an antibody. In certain embodiments, the antibody is atezolizumab. In one embodiment, atezolizumab is administered at about 1200 mg per dose.

In certain embodiments, the inhibitor of PD-1 or PD-L1 is administered at less than about 1500 mg/day, less than about 1200 mg/day, less than about 1000 mg/day, less than about 900 mg/day, less than about 800 mg/day, less than about 700 mg/day, less than about 600 mg/day, less than about 575 mg/day, less than about 550 mg/day, less than about 525 mg/day, less than about 500 mg/day, less than about 475 mg/day, less than about 450 mg/day, less than about 450 mg/day, less than about 425 mg/day, less than about 400 mg/day, less than about 375 mg/day, less than about 350 mg/day, less than about 325 mg/day, less than about 300 mg/day, less than about 275 mg/day, less than about 250 mg/day, or less than about 225 mg/day. In certain embodiments, the inhibitor is an inhibitor of PD-1. In certain embodiments, the inhibitor of PD-1 is an antibody. In certain embodiments, the antibody is pembrolizumab. In certain embodiments, the antibody is nivolumab. In certain embodiments, the inhibitor is an inhibitor of PD-L1. In certain embodiments, the inhibitor of PD-L1 is an antibody. In certain embodiments, the antibody is atezolizumab.

In certain embodiments of the methods disclosed herein, the inhibitor of PD-1 or PD-L1 is administered about every 1 to 24 weeks, about 1 to 20 weeks, about 1 to 19 weeks, about 1 to 18 weeks, about 1 to 17 weeks, about 1 to 16 weeks, about 1 to 15 weeks, about 1 to 14 weeks, about 1 to 13 weeks, about 1 to 12 weeks, about 1 to 10 weeks, about 1 to 9 weeks, about 1 to 8 weeks, about 1 to 7 weeks, about 1 to 6 weeks, about 1 to 5 weeks, about 1 to 4 weeks, about 1 to 3 weeks, or about 1 to 2 weeks. In certain embodiments, the inhibitor of PD-1 or PD-L1 is administered every week. In certain embodiments, the inhibitor of PD-1 or PD-L1 is administered every 2, 3, 4, 5, 6, 7, 8, 9, 10, 11, 12, 13, 14, 15, 16, 17, 18, 19, 20, 21, 22, 23, or 24 weeks. In certain embodiments, the inhibitor of PD-1 or PD-L1 is administered every 3 weeks. In certain embodiments, the inhibitor is an inhibitor of PD-1. In certain embodiments, the inhibitor of PD-1 is an antibody. In certain embodiments, the antibody is pembrolizumab. In certain embodiments, the antibody is nivolumab. In certain embodiments, the inhibitor is an inhibitor of PD-L1. In certain embodiments, the inhibitor of PD-L1 is an antibody. In certain embodiments, the antibody is atezolizumab.

In certain embodiments, the methods of the invention comprise the administration of at least two doses of the inhibitor of PD-1 or PD-L1 before the first administration of the T cell activation therapeutic. In conjunction with these embodiments, the inhibitor may additionally be administered to the subject at any other time before, during, or after the course of treatment with the T cell activation therapeutic, so long as at least two doses are administrated prior to a first administration of the T cell activation therapeutic.

In certain embodiments, the methods of the invention comprise the administration of at least two doses of the inhibitor of PD-1 or PD-L1 after the first administration of the T cell activation therapeutic. In conjunction with these embodiments, the inhibitor may additionally be administered to the subject at any other time during or after the course of treatment with the T cell activation therapeutic, so long as at least two doses are administrated after a first administration of the T cell activation therapeutic.

In an embodiment, the at least two doses include between 2-50 doses, more particularly between 2-36 doses, and more particularly between 2-18 doses. In an embodiment, the at least two doses are 2, 3, 4, 5, 6, 7, 8, 9, 10, 11, 12, 13 or 14 doses. The at least two doses may be separated by any suitable amount of time. In certain embodiments, the at least two doses comprise daily dose(s). In certain embodiments, the daily dose(s) are given everyday during the time in which the subject is treated for the tumor.

In certain embodiments, the methods of the invention involve administering at least two doses of an inhibitor of PD-1 or PD-L1, and then subsequently administering a T cell activation therapeutic of the invention (i.e., the administration of the inhibitor of PD-1 or PD-L1 starts before the first administration of the T cell activation therapeutic (e.g., at least two doses of inhibitor are given to the subject before the T cell activation therapeutic)). However, as described herein, the administering of the subject with the inhibitor of PD-1 or PD-L1 may continue after administration with the T cell activation therapeutic begins. In alternate embodiments, the administration of the inhibitor of PD-1 or PD-L1 stops before the first administration of the T cell activation therapeutic.

In certain methods of the invention, the first dose of an inhibitor of PD-1 or PD-L1 precedes any treatment of the subject with the T cell activation therapeutic. In an embodiment, the minimum amount of time separating the first administration of the inhibitor of PD-1 or PD-L1 and the first administration of the T cell activation therapeutic may be any amount of time sufficient to provide an immune-modulating effect. The skilled artisan will appreciate and take into consideration the amount of time sufficient to provide an immune-modulating based on the inhibitor of PD-1 or PD-L1 and the subject.

In some embodiments, the first dose of an inhibitor of PD-1 or PD-L1 is administered at least 12 hours before the first administration of the T cell activation therapeutic, and preferably at least two, four or six days before the first administration of the T cell activation therapeutic. In a further embodiment, the first dose of the inhibitor of PD-1 or PD-L1 may be provided about 1, 2, 3, 4, 5, 6, 7, 8, 9, 10, 11, 12, 13 or 14 days, or more, before the first administration of the T cell activation therapeutic. In a particular embodiment, the first administration of the inhibitor of PD-1 or PD-L1 occurs 1-4 days prior to the first administration of the T cell activation therapeutic. In certain embodiments, the first administration of the inhibitor of PD-1 or PD-L1 occurs about one week before the first administration of the T cell activation therapeutic.

In certain embodiments, the methods of the invention involve administering at least two doses of an inhibitor of PD-1 or PD-L1, after administration of a T cell activation therapeutic of the invention occurs (i.e., the administration of the T cell activation therapeutic starts before the first administration of the inhibitor of PD-1 or PD-L1).

In certain methods of the invention, the first dose of the T cell activation therapeutic precedes any treatment of the subject with the inhibitor of PD-1 or PD-L1. In an embodiment, the minimum amount of time separating the first administration of the cell activation therapeutic and the first administration of the inhibitor of PD-1 or PD-L1 may be any amount of time sufficient to provide an immune-modulating effect. The skilled artisan will appreciate and take into consideration the amount of time sufficient to provide an immune-modulating based on the inhibitor of PD-1 or PD-L1 and the subject.

In some embodiments, the first dose of an inhibitor of PD-1 or PD-L1 is administered at least 12 hours or 24 hours after the first administration of the T cell activation therapeutic. In a further embodiment, the first dose of the inhibitor of PD-1 or PD-L1 may be provided about 1, 2, 3, 4, 5, 6, 7, 8, 9, 10, 11, 12, 13 or 14 days, or more, after the first administration of the T cell activation therapeutic. In a particular embodiment, the first administration of the inhibitor of PD-1 or PD-L1 occurs 1-4 days after the first administration of the T cell activation therapeutic.

After the first dose with the inhibitor of PD-1 or PD-L1, subsequent doses may be administered at any desired interval of time between doses. In certain embodiments, the dosing with the inhibitor of PD-1 or PD-L1 may be stopped before, during, or after the course of treatment with the T cell activation therapeutic. In certain embodiments, the dosing with the inhibitor of PD-1 or PD-L1 may continue during the course of treatment with the T cell activation therapeutic.

In an embodiment, the first dose is of the inhibitor of PD-1 or PD-L1 followed by one or more maintenance doses (i.e., a dose of the inhibitor of PD-1 or PD-L1 that is given at such an interval and/or amount so as to maintain a sufficient amount of the inhibitor, and/or its active metabolites, in the body of the subject (e.g., avoid total systemic clearance thereof of the inhibitor and/or its active metabolites)). By providing a maintenance dose, it may be possible to prolong and/or maintain the immune-modulating effect of the inhibitor for an extended period of time before, during and/or after the course of administration with the T cell activation therapeutic.

In certain embodiments, for maintaining the immune-modulating effect, the inhibitor of PD-1 or PD-L1 may be administered 1, 2, 3, 4, or 5 times daily, or more. In certain embodiments, for maintaining the immune-modulating effect, the inhibitor of PD-1 or PD-L1 may be administered 1, 2, 3, 4, or 5 times daily, or more, so long as low dose administration is maintained (e.g., the multiple smaller doses add up to the desired daily low dose). In certain embodiments, for maintaining the immune-modulating effect, the inhibitor of PD-1 or PD-L1 may be administered once a week, once every two weeks, once every three weeks, once every four weeks, once every five weeks, once every six weeks, once every seven weeks, once every eight weeks, once every nine weeks, once every 10 weeks, once every 11 weeks, once every 12 weeks, once every four months, once every five months, once every six months, once every seven months, once every eight months, once every nine months, once every 10 months, once every 11 months, or once every 12 months. In certain embodiments, for maintaining the immune-modulating effect, the inhibitor of PD-1 or PD-L1 may be administered every about 1 to about 15 weeks, about 1 to about 14 weeks, about 1 to about 13 weeks, about 1 to about 12 weeks about 1 to about 11 weeks, about 1 to about 10 weeks, about 1 to about 9 weeks, about 1 to about 8 weeks, about 1 to about 7 weeks, about 1 to about 6 weeks, about 1 to about 5 weeks, about 1 to about 4 weeks, about 1 to about 3 weeks, about 1 to about 2 weeks. A single dose (i.e., administration) of the inhibitor of PD-1 or PD-L1 may be given at a single point in time, such as for example a pill that is swallowed. Alternatively, a single dose of the inhibitor of PD-1 or PD-L1 may be given over a short continuous period, such as for example by drip intravenous. The skilled person in the art would know or could determine, by routine skill, the appropriate interval for maintenance doses of the inhibitor of PD-1 or PD-L1.

In a particular embodiment, the inhibitor of PD-1 or PD-L1 is administered for a period of at least two consecutive days prior to or after the first administration of the T cell activation therapeutic. On these days, the inhibitor of PD-1 or PD-L1 may be administered to the subject at least 1, 2, 3, or 4 times daily, or any desired number of times to provide the daily low dose amount of the inhibitor.

In another embodiment, the inhibitor of PD-1 or PD-L1 is administered for a period of about one week prior to the first administration of the T cell activation therapeutic. In another embodiment, the inhibitor of PD-1 or PD-L1 is administered during the duration of treatment with the T cell activation therapeutic. Multiple doses may be provided during the treatment period. In exemplary embodiments, the inhibitor of PD-1 or PD-L1 may be administered every day, on every second day, or at any suitable interval for providing the described dosing.

In the methods of the invention, there may be a break in treatment with the inhibitor of PD-1 or PD-L1 before the first administration of the T cell activation therapeutic. In such embodiments, administration of the inhibitor of PD-1 or PD-L1 may be permanently or temporarily stopped before or after the first administration of the T cell activation therapeutic. The period of time between the last dose of the inhibitor of PD-1 or PD-L1 and the first dose of the T cell activation therapeutic may be any suitable period of time so long as the subject still obtains an immune-modulating benefit from the inhibitor.

In an alternate embodiment, treatment of the subject with the inhibitor of PD-1 or PD-L1 continues throughout the course of treatment with the T cell activation therapeutic, with or without intermittent breaks in the administration of the inhibitor. In further embodiments, treatment with the inhibitor of PD-1 or PD-L1 may continue after treatment with the T cell activation therapeutic ceases.

As described herein, treatment with the inhibitor of PD-1 or PD-L1 may be continued after the first administration with the T cell activation therapeutic. In an embodiment, administration of the inhibitor of PD-1 or PD-L1 is continued on a daily basis, with or without intermittent breaks, throughout the course of treatment with the T cell activation therapeutic. Therefore, in some embodiments, the inhibitor will be administered prior to and during the treatment with the T cell activation therapeutic. In such instances, once administration of the T cell activation therapeutic begins, it is possible for the inhibitor of PD-1 or PD-L1 to be administered at the same time as the T cell activation therapeutic, immediately sequentially, or at different times in the day. When the inhibitor of PD-1 or PD-L1 is administered at the same time as the T cell activation therapeutic, it may be included in the T cell activation therapeutic composition of the invention as a single composition or administered in a separate composition.

Alternatively, administration of the inhibitor of PD-1 or PD-L1 may be suspended during the days when the T cell activation therapeutic is administered. Therefore, regimens of the present invention may include taking a break in the administration of the T cell activation therapeutic during the course of administration of the T cell activation therapeutic.

In certain embodiments, administering the inhibitor of PD-1 or PD-L1 prior to the first administration of the T cell activation therapeutic applies also to the administration of the inhibitor after the first administration of the T cell activation therapeutic (e.g., before each subsequent administration of the T cell activation therapeutic).

In certain embodiments, the method of the invention comprises metronomic treatment of the subject with the inhibitor of PD-1 or PD-L1. In an embodiment of the methods of the present invention, metronomic treatment with the inhibitor of PD-1 or PD-L1 is intended to encompass a daily low dose administration of the inhibitor over a certain period of time, such as for example a period of 2, 3, 4, 5, 6 or 7, or more, consecutive days. During these days of metronomic dosing, the inhibitor of PD-1 or PD-L1 may be provided at frequent regular intervals or varying intervals. For example, in an embodiment, a dose of the inhibitor of PD-1 or PD-L1 may be administered every 1, 2, 3, 4, 6, 8, 12 or 24 hours. In another embodiment, a dose of the inhibitor of PD-1 or PD-L1 may be administered every 2, 3, or 4 days.

In some embodiments of the methods of the present invention, there may be breaks or gaps in the periods of metronomic treatment with the inhibitor of PD-1 or PD-L1. In this manner, metronomic treatment with the inhibitor of PD-1 or PD-L1 may occur in a cyclic fashion, alternating between on and off periods of administration. Particularly suitable are intervals where the inhibitor of PD-1 or PD-L1 is administered to the subject daily on alternating weekly intervals. For instance, a one-week period of administration of the inhibitor of PD-1 or PD-L1 is followed by a one-week suspension of treatment, and the cycle repeats.

In an embodiment therefore, the methods of the invention comprise administering the inhibitor of PD-1 or PD-L1 to the subject daily during the course of tumor treatment. In certain embodiments, the administration of the inhibitor of PD-1 or PD-L1 begins about 1, 2, 3, 4, 5, 6, 7, 8, 9, 10, 11, 12, 13, or 14 days after the first administration of the T cell activation therapeutic. In a particular aspect of this embodiment, the administration of the inhibitor of PD-1 or PD-L1 begins about 1 day after the first administration of the T cell activation therapeutic.

As the skilled person will appreciate, the frequency and duration of the administration of the inhibitor of PD-1 or PD-L1 and the survivin therapeutic may be adjusted as desired for any given subject. Factors that may be taken into account include, e.g.: the nature of the one or more survivin antigens in the survivin therapeutic; the type of cancer; the age, physical condition, body weight, sex and diet of the subject; and other clinical factors.

The inhibitor of PD-1 or PD-L1 may be administered by any suitable delivery means and any suitable route of administration. In an embodiment, the inhibitor of PD-1 or PD-L1 is administered orally, such as in the form of a pill, tablet, or capsule. In an alternate embodiment, the inhibitor is administered by injection (e.g., intravenous).

In certain embodiments, the active inhibitor is administered before, at the same time, and/or after the administration of the T cell activation therapeutic.

The active agent is typically administered in an amount sufficient to provide an immune-modulating effect.

In certain embodiments, the active agent is administered at a dose of about 5 mg to about 5 g, about 10 mg to about 4.5 g, about 15 mg to about 4 g, about 20 mg to about 3.5 g, about 25 mg to about 3 g, about 30 mg to about 2.5 g, about 35 mg to about 2 g, about 40 mg to about 1.5 g, about 45 mg to about 1 g, about 50 mg to about 900 mg, about 55 mg to about 850 mg, about 60 mg to about 800 mg, about 65 mg to about 750 mg, about 70 mg to about 700 mg, about 75 mg to about 650 mg, about 80 mg to about 600 mg, about 85 mg to about 550 mg, about 90 mg to about 500 mg, about 95 mg to about 450 mg, about 100 mg to about 400 mg, about 110 mg to about 350 mg, about 120 mg to about 300 mg, about 130 mg to about 290 mg, about 140 mg to about 280 mg, about 150 mg to about 270 mg, about 160 mg to about 260 mg, about 170 mg to about 250 mg, about 180 mg to about 240 mg, about 190 mg to about 230 mg, or about 200 mg to about 220 mg. In certain embodiments, the active agent is administered at a dose of at least about 5 mg, at least about 10 mg, at least about 15 mg, at least about 20 mg, at least about 25 mg, at least about 30 mg, at least about 40 mg, at least about 50 mg, at least about 60 mg, at least about 70 mg, at least about 75 mg, at least about 80 mg, at least about 90 mg, at least about 100 mg, at least about 125 mg, at least about 150 mg, at least about 175 mg, at least about 200 mg, at least about 225 mg, at least about 250 mg, at least about 275 mg, at least about 300 mg, at least about 325 mg, at least about 350 mg, at least about 375 mg, at least about 400 mg, at least about 425 mg, at least about 450 mg, at least about 475 mg, at least about 500 mg, at least about 525 mg, at least about 550 mg, at least about 575 mg, at least about 600 mg, at least about 625 mg, at least about 650 mg, at least about 675 mg, at least about 700 mg, at least about 725 mg, at least about 750 mg, at least about 775 mg, at least about 800 mg, at least about 825 mg, at least about 850 mg, at least about 875 mg, at least about 900 mg, at least about 925 mg, at least about 950 mg, at least about 975 mg, at least about 1 g, at least about 2 g, at least about 3 g, at least about 4 g, or at least about 5 g.

In certain embodiments, the “amount sufficient to provide an immune-modulating effect” may be a “low dose” amount. Thus, in certain embodiments, the methods of the invention involve the use of a low dose of an active agent that in combination with the T cell activation therapeutic.

As it relates to certain embodiments of the invention “low dose” may refer to a dose of active agent that is less than about 300 mg/m², such as for example about 100-300 mg/m². In terms of daily administration, a “low dose” of active agent is between about 25-300 mg/day or about 50-150 mg/day. In certain embodiments, a daily dosage amount is about 100 mg of active agent. In certain embodiments, a daily dosage amount is about 50 mg of active agent per dose.

As it relates to certain embodiments of the invention wherein the active agent is the alkylating agent cyclophosphamide, the expression “low dose” typically refers to a dose of cyclophosphamide that is less than about 300 mg/m², such as for example about 100-300 mg/m². In terms of daily administration, a “low dose” of cyclophosphamide is between about 25-300 mg/day or about 50-150 mg/day. In certain embodiments, a daily dosage amount is about 100 mg of cyclophosphamide. In certain embodiments, a daily dosage amount is about 50 mg of cyclophosphamide per dose. In some embodiments, cyclophosphamide enhances survivin-based T cell responses.

The “low dose” amounts of other active agents, as encompassed herein, would be known to those skilled in the art, or could be determined by routine skill.

In certain embodiments, the methods of the invention comprise the administration of at least two doses of the active agent before the first administration of the T cell activation therapeutic. In conjunction with these embodiments, the active agent may additionally be administered to the subject at any other time before, during, or after the course of treatment with the T cell activation therapeutic, so long as at least two doses are administrated prior to a first administration of the T cell activation therapeutic.

As used herein, the expression “at least two doses” is intended to encompass any number of doses that is greater than a single dose. In an embodiment, the at least two doses include between 2-50 doses, more particularly between 2-28 doses, and more particularly between 2-14 doses. In an embodiment, the at least two doses are 2, 3, 4, 5, 6, 7, 8, 9, 10, 11, 12, 13 or 14 doses. The at least two doses may be separated by any suitable amount of time. In a particular embodiment, the at least two doses comprise 2 doses daily for a period of one week, totaling 14 doses.

In certain embodiments, the methods of the invention involve administering at least two doses of an active agent, and then subsequently administering a T cell activation therapeutic of the invention. By “subsequently administering”, it is meant that the administration of the active agent starts before the first administration of the T cell activation therapeutic (e.g., at least one or at least two doses of agent are given to the subject before the T cell activation therapeutic). However, as described herein, the administering of the active agent to the subject may continue after administration with the T cell activation therapeutic begins. In alternate embodiments, the administration of the active agent stops before the first administration of the T cell activation therapeutic.

In certain embodiments, the methods of the invention are such that the first dose of an active agent precedes any treatment of the subject with the T cell activation therapeutic. In an embodiment, the minimum amount of time separating the first administration of the active agent and the first administration of the T cell activation therapeutic may be any amount of time sufficient to provide an immune-modulating effect. The skilled artisan will appreciate and take into consideration the amount of time sufficient to provide an immune-modulating based on the active agent and the subject.

In some embodiments, the first dose of an active agent is administered at least 12 hours before the first administration of the T cell activation therapeutic, and preferably at least two, four or six days before the first administration of the T cell activation therapeutic. In a further embodiment, the first dose of the active agent may be provided about 1, 2, 3, 4, 5, 6, 7, 8, 9, 10, 1, 12, 13 or 14 days, or more, before the first administration of the T cell activation therapeutic. In a particular embodiment, the first administration of the active agent occurs 1-4 days prior to the first administration of the T cell activation therapeutic. In certain embodiments, the first administration of the active agent occurs about one week before the first administration of the T cell activation therapeutic.

After the first dose of the active agent, subsequent doses may be administered at any desired interval of time between doses, so long as at least two doses of the agent are administered before the first administration of the T cell activation therapeutic. The dosing with the active agent may be stopped before, during or after the course of treatment with the T cell activation therapeutic.

In an embodiment, the first dose of the active agent may be followed by one or more maintenance doses. As used herein, the term “maintenance dose” is meant to encompass a dose of the active agent that is given at such an interval and/or amount so as to maintain a sufficient amount of the agent, and/or its active metabolites, in the body of the subject (e.g., avoid total systemic clearance thereof of the agent and/or its active metabolites). By providing a maintenance dose, it may be possible to prolong and/or maintain the immune-modulating effect of the active agent for an extended period of time before, during, and/or after the course of administration with the T cell activation therapeutic.

In certain embodiments, for maintaining the immune-modulating effect, the active agent may be administered 1, 2, 3, 4 or 5 times daily, or more. In certain embodiments, for maintaining the immune-modulating effect, the active agent may be administered 1, 2, 3, 4 or 5 times daily, or more so long as low dose administration is maintained (e.g., the multiple smaller doses add up to the desired daily low dose). A single dose (i.e., administration) of the active agent may be given at a single point in time, such as for example a pill that is swallowed. Alternatively, a single dose of the active agent may be given over a short continuous period, such as for example by drip intravenous.

For embodiments of the invention where the active agent is cyclophosphamide, it may be appropriate to provide a maintenance dose, for example, every 6-18 hours. The skilled person in the art would know or could determine, by routine skill, the appropriate interval for maintenance doses of cyclophosphamide, as well as for other active agents as encompassed herein.

In a particular embodiment, the active agent is administered for a period of at least two consecutive days prior to the first administration of the T cell activation therapeutic. On these days, the active agent may be administered to the subject at least 1, 2, 3 or 4 times daily, or any desired number of times. In certain embodiments, the active agent is administered to the subject at least 1, 2, 3 or 4 times daily, or any desired number of times to provide the daily low dose amount of the agent.

In another embodiment, the active agent is administered for a period of about one week prior to the first administration of the T cell activation therapeutic. Multiple doses may be provided during this one-week period. In exemplary embodiments, the active agent may be administered every day, on every second day, or at any suitable interval for providing the described maintenance dose. For example, in certain embodiments of the method of the invention comprises administering the active agent twice daily for a period of about one week prior to administering the T cell activation therapeutic.

In the methods of the invention, there may be a break in treatment with the active agent before the first administration of the T cell activation therapeutic. In such embodiments, administration of the active agent may be permanently or temporarily stopped before the first administration of the T cell activation therapeutic. The period of time between the last dose of the active agent and the first dose of the T cell activation therapeutic may be any suitable period of time so long as the subject still obtains an immune-modulating benefit from the agent. For example, and without limitation, the administration of the active agent may be stopped at the same time that the first dose of T cell activation therapeutic is administered or at any time up to about one week before the first dose of the T cell activation therapeutic. For example, and without limitation, administration of the active agent may be stopped at about 6, 12, 18, 24, 36, 48, 60 or 72 hours, or more, before the first dose of the T cell activation therapeutic. In certain embodiments, administration of the active agent is stopped about 2, 4 or 7 days before the first dose of the T cell activation therapeutic.

In an alternate embodiment, treatment of the subject with the active agent continues throughout the course of treatment with the T cell activation therapeutic, with or without intermittent breaks in the administration of the agent. In further embodiments, treatment with the active agent may continue after treatment with the T cell activation therapeutic ceases. Thus, in an embodiment, the active agent may be administered during the period before each administration with the T cell activation therapeutic. Alternatively, the active agent may only be administered during the period before the first administration with the T cell activation therapeutic.

As described herein, treatment with the active agent may be continued after the first administration with the T cell activation therapeutic. In an embodiment, administration of the active agent is continued on a daily basis, with or without intermittent breaks, throughout the course of treatment with the T cell activation therapeutic. Therefore, in some embodiments, the agent will be administered prior to and during the treatment with the T cell activation therapeutic. In such instances, once administration of the T cell activation therapeutic begins, it is possible for the active agent to be administered at the same time as the T cell activation therapeutic, immediately sequentially, or at different times in the day. When the active agent is administered at the same time as the T cell activation therapeutic, it may be included in the T cell activation therapeutic composition of the invention as a single composition or administered in a separate composition.

Alternatively, administration of the active agent may be suspended during the days when the T cell activation therapeutic is administered. Therefore, regimens of the present invention may include taking a break in the administration of the ag T cell activation therapeutic during the course of administration of the T cell activation therapeutic.

The embodiments described herein for administering the active agent prior to the first administration of the T cell activation therapeutic apply also to the administration of the agent after the first administration of the T cell activation therapeutic (e.g., before each subsequent administration of the T cell activation therapeutic).

In certain embodiments, the method of the invention comprises metronomic treatment of the subject with the active agent. For purposes of the present invention, “metronomic treatment”, “metronomic regimen”, “metronomic dosing” or “low-dose intermittent” or the like, is meant to refer to a frequent administration of a lower than normal dose amount of the agent that interferes with DNA replication. As used herein, the term “normal dose amount” may refer, for example and without limitation, to either: (i) the established maximum tolerated dose (MTD) or standard dose via a traditional dosing schedule, or (ii) in instances where a low dose single bolus amount has been established for a particular active agent, than to that low dose amount.

In metronomic dosing, the same, lower, or higher cumulative dose over a certain time period as would be administered via a traditional dosing schedule may ultimately be administered. In a particularly suitable embodiment, this is achieved by extending the time frame during which the dosing is conducted and/or increasing the frequency of administrations, while decreasing the amount administered as compared to the normal dose amount. For example, where a low dose amount of 300 mg/m² of an active agent is typically administered (e.g., by single bolus injection), a metronomic regimen may comprise administering the same amount over a period of several days by administering frequent low doses. By this approach, metronomic dosing may be used, for example, to provide the maintenance doses as described herein.

In an embodiment of the methods of the present invention, metronomic treatment with the active agent is intended to encompass a daily low dose administration of the agent over a certain period of time, such as for example a period of 2, 3, 4, 5, 6 or 7, or more, consecutive days. During these days of metronomic dosing, the active agent may be provided at frequent regular intervals or varying intervals. For example, in an embodiment, a dose of the active agent may be administered every 1, 2, 3, 4, 6, 8, 12 or 24 hours. In another embodiment, a dose of the active agent may be administered every 2, 3, or 4 days.

In some embodiments of the methods of the present invention, there may be breaks or gaps in the periods of metronomic treatment with the active agent. In this manner, metronomic treatment with the active agent may occur in a cyclic fashion, alternating between on and off periods of administration. Particularly suitable are intervals where the active agent is administered to the subject daily on alternating weekly intervals. For instance, a one-week period of administration of the active agent is followed by a one-week suspension of treatment, and the cycle repeats.

In an embodiment, the methods of the invention comprise administering the active agent to the subject daily for a period of one week every second week. In a particular aspect of this embodiment, the administration of the active agent begins about one week before the first administration of the T cell activation therapeutic.

As it relates to the T cell activation therapeutic of the invention, in some embodiments it may be suitable to administer the T cell activation therapeutic to the subject at an interval of once every week, once every two weeks, once every three weeks, once every four weeks, once every five weeks, once every six weeks, once every seven weeks, once every eight weeks, once every nine weeks, once every ten weeks, once every eleven weeks, once every twelve weeks, once every thirteen weeks, once every fourteen weeks, or once every fifteen weeks. In certain embodiments, the T cell activation therapeutic is administered once every three weeks. In certain embodiments, the T cell activation therapeutic is administered once every six weeks to once every twelve weeks. The frequency and duration of the administration of the T cell activation therapeutic may however be adjusted as desired for any given subject and may be more or less frequent than once every week, once every two weeks or once every three weeks. The interval between the administrations may also not be constant during the course of treatment with the T cell activation therapeutic. In the methods of the invention, the T cell activation therapeutic may be administered to the subject 1, 2, 3, 4, 5, 6, 7, 8, 9, 10 or more times. It will be understood that treatment with the T cell activation therapeutic may be continued for an indefinite period depending on how the treatment of the tumor in the subject is progressing. As it relates to the T cell activation therapeutic of the invention, in some embodiments it may be suitable to administer the T cell activation therapeutic to the subject as adjuvant or neoadjuvant treatment. As it relates to the T cell activation therapeutic of the invention, in some embodiments it may be suitable to administer the T cell activation therapeutic to the subject as adjuvant and as neoadjuvant treatment.

In some embodiments, the methods of the present disclosure can be use as an adjuvant treatment. As used herein “adjuvant treatment” refers to any additional cancer treatment given after the primary treatment. In some embodiments, adjuvant treatment is given to lower the risk that the cancer will come back. Adjuvant therapy may include, but not limited to chemotherapy, radiation therapy, hormone therapy, targeted therapy, biological therapy or combinations thereof.

In some embodiments, the methods of the present disclosure can be use as a neoadjuvant treatment. As used herein “neoadjuvant treatment” refers to any treatment given as a first step to shrink a tumor before the main treatment, which is usually surgery, is given. Neoadjuvant therapy may include, but not limited to, chemotherapy, radiation therapy, hormone therapy or combinations thereof.

In some embodiments, the methods of the present disclosure can be use as a consolidation therapy. As used herein “consolidation therapy” refers to any treatment that is given after cancer has disappeared following the initial therapy. In some embodiments, consolidation therapy is used to kill any cancer cells that may be left in the body. In some embodiments, consolidation therapy can include, but not limited to, radiation therapy, a stem cell transplant, treatment with drugs that kill cancer cells or combinations thereof. Also called intensification therapy and postremission therapy.

In some embodiments, the methods of the present disclosure can be use as a maintenance therapy. As used herein “maintenance therapy” refers to any that is given to help keep cancer from coming back after it has disappeared following the initial therapy. In some embodiments, maintenance therapy can include, but not limited to, treatment with drugs, vaccines, or antibodies that kill cancer cells, or combinations thereof and it may be given for a long time.

In certain embodiments it may be suitable to administer the T cell activation therapeutic to the subject in the neoadjuvant phase at an interval of once every week, once every two weeks, once every three weeks, once every four weeks, once every five weeks, once every six weeks, once every seven weeks, once every eight weeks, once every nine weeks, once every ten weeks, once every eleven weeks, once every twelve weeks, once every thirteen weeks, once every fourteen weeks, or once every fifteen weeks. In certain embodiments it may be suitable to administer the T cell activation therapeutic to the subject in the neoadjuvant phase at an interval of once every week, once every two weeks, once every three weeks, once every four weeks, once every five weeks, or once every six weeks. In certain embodiments it may be suitable to administer the T cell activation therapeutic to the subject in the neoadjuvant phase at an interval of once every three weeks.

In certain embodiments it may be suitable to administer the T cell activation therapeutic to the subject in the adjuvant phase at an interval of once every week, once every two weeks, once every three weeks, once every four weeks, once every five weeks, once every six weeks, once every seven weeks, once every eight weeks, once every nine weeks, once every ten weeks, once every eleven weeks, once every twelve weeks, once every thirteen weeks, once every fourteen weeks, or once every fifteen weeks. In certain embodiments it may be suitable to administer the T cell activation therapeutic to the subject in the adjuvant phase at an interval of once every four weeks, once every five weeks, once every six weeks, once every seven weeks, once every eight weeks, once every nine weeks, once every ten weeks, once every eleven weeks, once every twelve weeks, once every thirteen weeks, once every fourteen weeks, or once every fifteen weeks. In certain embodiments it may be suitable to administer the T cell activation therapeutic to the subject in the adjuvant phase at an interval of once every six weeks, once every seven weeks, once every eight weeks, once every nine weeks, once every ten weeks, once every eleven weeks, or once every twelve weeks.

In certain embodiments it may be suitable to administer the T cell activation therapeutic to the subject in the neoadjuvant phase at an interval of once every week, once every two weeks, once every three weeks, once every four weeks, once every five weeks, once every six weeks. In certain embodiments it may be suitable to administer the T cell activation therapeutic to the subject in the neoadjuvant phase at an interval of once every three weeks. In certain embodiments it may be suitable to administer the T cell activation therapeutic to the subject in the adjuvant phase at an interval of once every four weeks, once every five weeks, once every six weeks, once every seven weeks, once every eight weeks, once every nine weeks, once every ten weeks, once every eleven weeks, once every twelve weeks, once every thirteen weeks, once every fourteen weeks, or once every fifteen weeks. In certain embodiments it may be suitable to administer the T cell activation therapeutic to the subject in the adjuvant phase at an interval of once every six weeks, once every seven weeks, once every eight weeks, once every nine weeks, once every ten weeks, once every eleven weeks, or once every twelve weeks.

In certain embodiments it may be suitable to administer the T cell activation therapeutic to the subject in the neoadjuvant phase at an interval of once every week, once every two weeks, once every three weeks, once every four weeks, once every five weeks, once every six weeks and in the adjuvant phase at an interval of once every four weeks, once every five weeks, once every six weeks, once every seven weeks, once every eight weeks, once every nine weeks, once every ten weeks, once every eleven weeks, once every twelve weeks, once every thirteen weeks, once every fourteen weeks, or once every fifteen weeks.

In certain embodiments it may be suitable to administer the T cell activation therapeutic to the subject in the neoadjuvant phase at an interval of once every three weeks and in the adjuvant phase at an interval of once every six weeks, once every seven weeks, once every eight weeks, once every nine weeks, once every ten weeks, once every eleven weeks, or once every twelve weeks.

In certain embodiments, the T cell activation therapeutic is administered at a dose of about 5 μg to about 1000 μg, about 10 μg to about 950 μg, about 15 μg to about 900 μg, about 20 μg to about 850 μg, about 25 μg to about 800 μg, about 30 μg to about 750 μg, about 35 μg to about 700 μg, about 40 μg to about 650 μg, about 45 μg to about 600 μg, about 50 μg to about 550 μg, about 55 μg to about 500 μg, about 60 μg to about 450 μg, about 65 μg to about 400 μg, about 65 μg to about 350 μg, about 70 μg to about 300 μg, about 75 μg to about 275 μg, about 80 μg to about 250 μg, about 85 μg to about 225 μg, about 90 μg to about 200 μg, about 95 μg to about 175 μg, or about 100 μg to about 150 μg. In certain embodiments, the T cell activation therapeutic is administered at a dose of about 50 μg to about 500 μg, about 50 μg to about 100 μg, about 60 μg to about 90 μg, 70 μg to about 80 μg, about 100 μg to about 500 μg, about 120 μg to about 480 μg, about 140 μg to about 460 μg, about 160 μg to about 440 μg, about 180 μg to about 420 μg, about 200 μg to about 400 μg, about 220 μg to about 380 μg, about 240 μg to about 360 μg, about 260 μg to about 340 μg, about 280 μg to about 320 μg, or about 300 μg to about 310 μg.

In an embodiment of the methods of the invention, the active agent may be administered as a priming agent during the intermittent period before each administration of the T cell activation therapeutic.

In a particular embodiment, a method of the invention comprising the combination of an active agent and a survivin therapeutic will involve the survivin therapeutic being administered to the subject at an interval of once every three weeks (e.g., Day 0, 21, 42, 63, 84, etc.) with the first administration the active agent beginning about one week before (e.g., Day -7) the first survivin therapeutic administration and the continuing daily (e.g., metronomic) on alternating weekly intervals.

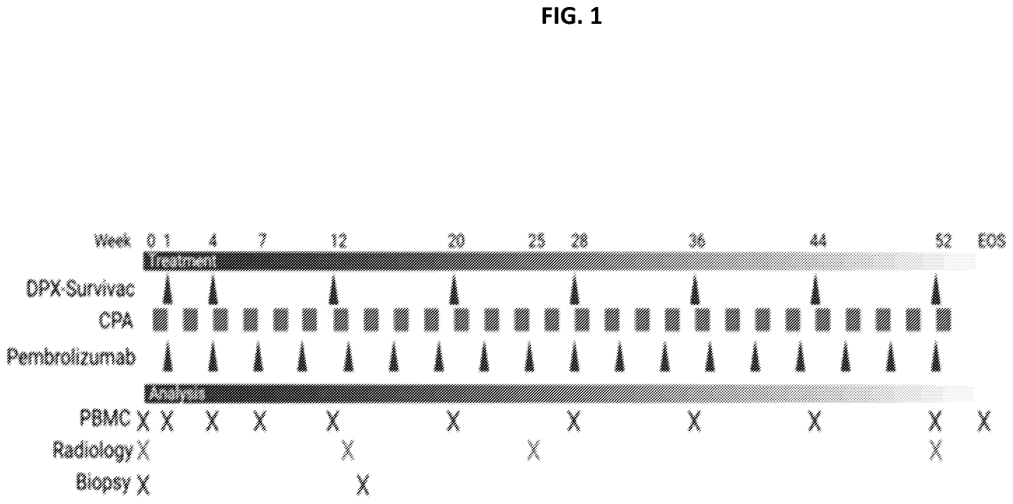

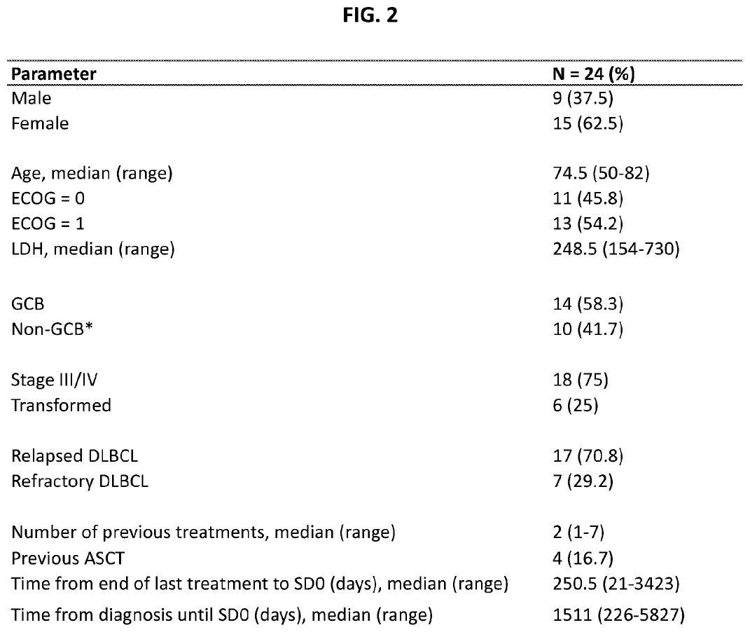

In a particular embodiment, a method of the invention comprising the combination of an active agent and a survivin therapeutic will involve the first two doses of survivin therapeutic being administered to the subject three weeks apart (e.g., Week 1 and 4) and any subsequent doses being administered at an interval of once every eight weeks (e.g., Week 12, 20, 28, 36, 44, etc.) with the first administration the active agent beginning about one week before (e.g., Week 0) the first survivin therapeutic administration and the continuing daily (e.g., metronomic) on alternating weekly intervals. The method may also involve administering an inhibitor of PD-1 or PD-L1 every three weeks with the first dose starting the same week as the first survivin therapeutic administration. A treatment regime such as this is shown in FIG. 1.

As the skilled person will appreciate, the frequency and duration of the administration of the active agent and the survivin therapeutic may be adjusted as desired for any given subject. Factors that may be taken into account include, e.g.: the nature of the one or more survivin antigens in the survivin therapeutic, the type of cancer, the age, physical condition, body weight, sex and diet of the subject; and other clinical factors.

The active agent may be administered by any suitable delivery means and any suitable route of administration. In an embodiment, the active agent is administered orally, such as in the form of a pill, tablet or capsule. In an alternate embodiment, the agent is administered by injection (e.g., intravenous). In a particular embodiment of the methods of the invention, the agent is cyclophosphamide and it is administered orally.

The T cell activation therapeutic of the invention as described herein may be formulated in a form that is suitable for oral, nasal, rectal or parenteral administration. Parenteral administration includes intravenous, intraperitoneal, intradermal, subcutaneous, intramuscular, transepithelial, intrapulmonary, intrathecal, and topical modes of administration. In embodiments where the T cell activation therapeutic is formulated as a composition as described above so as to achieve a depot effect at the site of injection. The T cell activation therapeutic and the active agent are not necessarily administered by the same route of administration or at the same time.

In a particular embodiment of the methods of the invention, the active agent is an alkylating agent, such as for example cyclophosphamide.

In certain embodiments, an additional therapeutic agent is administered.

In certain embodiments, administration of the additional therapeutic agent and the inhibitor of PD-L1 or PD-1, T cell activation therapeutic, and/or active agent to a single patient and are intended to include instances wherein the agent and the inhibitor of PD-L1 or PD-1, T cell activation therapeutic, and/or active agent are not necessarily administered by the same route of administration or at the same time. For example, the additional therapeutic agent and the inhibitor of PD-L1 or PD-1, T cell activation therapeutic, and/or active agent may be administered separately, sequentially, or using alternating administration.

In certain embodiments, the active agent is administered before, at the same time, or after the administration of the T cell activation therapeutic.

The additional therapeutic agent is typically administered in an amount sufficient to provide an immune-modulating effect.

In some embodiments, the additional therapeutic agent may include one or more of Rituximab, obinutuzumab, Cyclophosphamide, Mitoxantrone, Vincristine, Prednisone, etoposide, Methotrexate/Ifosfamide and Cytarabine, Dexamethasone, Cisplatin, Gemcitabine, Brentuximab vedotin, Bendamustine, liposomal Doxorubicin, Lenalidomide, ibrutinib, selinixor, Polatuzumab vedotin, Pixantrone, CAR-T cells (e.g. Yescarta, Kymriah), mozunetuzumab, bispecific antibodies, thalidomide, Autologous Stem Cell Transplant, or tafasitamab.

In certain embodiments, the additional therapeutic agent is administered at a dose of about 10 mg to about 1 g, about 5 mg to about 5 g, about 10 mg to about 4.5 g, about 15 mg to about 4 g, about 20 mg to about 3.5 g, about 25 mg to about 3 g, about 30 mg to about 2.5 g, about 35 mg to about 2 g, about 40 mg to about 1.5 g, about 45 mg to about 1 g, about 50 mg to about 900 mg, about 55 mg to about 850 mg, about 60 mg to about 800 mg, about 65 mg to about 750 mg, about 70 mg to about 700 mg, about 75 mg to about 650 mg, about 80 mg to about 600 mg, about 85 mg to about 550 mg, about 90 mg to about 500 mg, about 95 mg to about 450 mg, about 100 mg to about 400 mg, about 110 mg to about 350 mg, about 120 mg to about 300 mg, about 130 mg to about 290 mg, about 140 mg to about 280 mg, about 150 mg to about 270 mg, about 160 mg to about 260 mg, about 170 mg to about 250 mg, about 180 mg to about 240 mg, about 190 mg to about 230 mg, or about 200 mg to about 220 mg. In certain embodiments, the additional therapeutic agent is administered at a dose of about 50 mg to about 350 mg, about 100 mg to about 300 mg, or about 150 mg to about 250 mg. In certain embodiments, the additional therapeutic agent is administered at a dose of or at least a dose of about 5 mg, at least about 10 mg, at least about 15 mg, at least about 20 mg, at least about 25 mg, at least about 30 mg, at least about 40 mg, at least about 50 mg, at least about 60 mg, at least about 70 mg, at least about 75 mg, at least about 80 mg, at least about 90 mg, at least about 100 mg, at least about 125 mg, at least about 150 mg, at least about 175 mg, at least about 200 mg, at least about 225 mg, at least about 250 mg, at least about 275 mg, at least about 300 mg, at least about 325 mg, at least about 350 mg, at least about 375 mg, at least about 400 mg, at least about 425 mg, at least about 450 mg, at least about 475 mg, at least about 500 mg, at least about 525 mg, at least about 550 mg, at least about 575 mg, at least about 600 mg, at least about 625 mg, at least about 650 mg, at least about 675 mg, at least about 700 mg, at least about 725 mg, at least about 750 mg, at least about 775 mg, at least about 800 mg, at least about 825 mg, at least about 850 mg, at least about 875 mg, at least about 900 mg, at least about 925 mg, at least about 950 mg, at least about 975 mg, at least about 1 g, at least about 2 g, at least about 3 g, at least about 4 g, or at least about 5 g.

In certain embodiments, the additional therapeutic agent is a CAR-T cell therapy administered at a dose of about 0.01×10⁶ CAR-positive viable T cells/kg, about 0.02×10⁶ CAR-positive viable T cells/kg, about 0.03×10⁶ CAR-positive viable T cells/kg, about 0.04×10⁶ CAR-positive viable T cells/kg, about 0.05×10⁶ CAR-positive viable T cells/kg, about 0.06×10⁶ CAR-positive viable T cells/kg, about 0.07×10⁶ CAR-positive viable T cells/kg, about 0.08×10⁶ CAR-positive viable T cells/kg, about 0.09×10⁶ CAR-positive viable T cells/kg, about 0.1×10⁶ CAR-positive viable T cells/kg, about 0.2×10⁶ CAR-positive viable T cells/kg, about 0.3×10⁶ CAR-positive viable T cells/kg, about 0.4×10⁶ CAR-positive viable T cells/kg, about 0.5×10⁶ CAR-positive viable T cells/kg, about 0.6×10⁶ CAR-positive viable T cells/kg, about 0.7×10⁶ CAR-positive viable T cells/kg, about 0.8×10⁶ CAR-positive viable T cells/kg, about 0.9×10⁶ CAR-positive viable T cells/kg, about 1.0×10⁶ CAR-positive viable T cells/kg, about 1.1×10⁶ CAR-positive viable T cells/kg, about 1.2×10⁶ CAR-positive viable T cells/kg, about 1.25×10⁶ CAR-positive viable T cells/kg, about 1.3×10⁶ CAR-positive viable T cells/kg, about 1.4×10⁶ CAR-positive viable T cells/kg, about 1.5×10⁶ CAR-positive viable T cells/kg, about 1.6×10⁶ CAR-positive viable T cells/kg, about 1.7×10⁶ CAR-positive viable T cells/kg, about 1.75×10⁶ CAR-positive viable T cells/kg, about 1.8×10⁶ CAR-positive viable T cells/kg, about 1.9×10⁶ CAR-positive viable T cells/kg, about 2.0×10⁶ CAR-positive viable T cells/kg, about 2.1×10⁶ CAR-positive viable T cells/kg, about 2.2×10⁶ CAR-positive viable T cells/kg, about 2.25×10⁶ CAR-positive viable T cells/kg, about 2.3×10⁶ CAR-positive viable T cells/kg, about 2.4×10⁶ CAR-positive viable T cells/kg, about 2.5×10⁶ CAR-positive viable T cells/kg, about 2.6×10⁶ CAR-positive viable T cells/kg, about 2.7×10⁶ CAR-positive viable T cells/kg, about 2.75×10⁶ CAR-positive viable T cells/kg, about 2.8×10⁶ CAR-positive viable T cells/kg, about 2.9×10⁶ CAR-positive viable T cells/kg, about 3.0×10⁶ CAR-positive viable T cells/kg, about 3.1×10⁶ CAR-positive viable T cells/kg, about 3.2×10⁶ CAR-positive viable T cells/kg, about 3.25×10⁶ CAR-positive viable T cells/kg, about 3.3×10⁶ CAR-positive viable T cells/kg, about 3.4×10⁶ CAR-positive viable T cells/kg, about 3.5×10⁶ CAR-positive viable T cells/kg, about 3.6×10⁶ CAR-positive viable T cells/kg, about 3.7×10⁶ CAR-positive viable T cells/kg, about 3.75×10⁶ CAR-positive viable T cells/kg, about 3.8×10⁶ CAR-positive viable T cells/kg, about 3.9×10⁶ CAR-positive viable T cells/kg, about 4.0×10⁶ CAR-positive viable T cells/kg, about 4.1×10⁶ CAR-positive viable T cells/kg, about 4.2×10⁶ CAR-positive viable T cells/kg, about 4.25×10⁶ CAR-positive viable T cells/kg, about 4.3×10⁶ CAR-positive viable T cells/kg, about 4.4×10⁶ CAR-positive viable T cells/kg, about 4.5×10⁶ CAR-positive viable T cells/kg, about 4.6×10⁶ CAR-positive viable T cells/kg, about 4.7×10⁶ CAR-positive viable T cells/kg, about 4.75×10⁶ CAR-positive viable T cells/kg, about 4.8×10⁶ CAR-positive viable T cells/kg, about 4.9×10⁶ CAR-positive viable T cells/kg, about 5.0×10⁶ CAR-positive viable T cells/kg, about 5.1×10⁶ CAR-positive viable T cells/kg, about 5.2×10⁶ CAR-positive viable T cells/kg, about 5.25×10⁶ CAR-positive viable T cells/kg, about 5.3×10⁶ CAR-positive viable T cells/kg, about 5.4×10⁶ CAR-positive viable T cells/kg, about 5.5×10⁶ CAR-positive viable T cells/kg, about 5.6×10⁶ CAR-positive viable T cells/kg, about 5.7×10⁶ CAR-positive viable T cells/kg, about 5.75×10⁶ CAR-positive viable T cells/kg, about 5.8×10⁶ CAR-positive viable T cells/kg, about 5.9×10⁶ CAR-positive viable T cells/kg, about 6.0×10⁶ CAR-positive viable T cells/kg, about 6.1×10⁶ CAR-positive viable T cells/kg, about 6.2×10⁶ CAR-positive viable T cells/kg, about 6.25×10⁶ CAR-positive viable T cells/kg, about 6.3×10⁶ CAR-positive viable T cells/kg, about 6.4×10⁶ CAR-positive viable T cells/kg, about 6.5×10⁶ CAR-positive viable T cells/kg, about 6.6×10⁶ CAR-positive viable T cells/kg, about 6.7×10⁶ CAR-positive viable T cells/kg, about 6.75×10⁶ CAR-positive viable T cells/kg, about 6.8×10⁶ CAR-positive viable T cells/kg, about 6.9×10⁶ CAR-positive viable T cells/kg, about 7.0×10⁶ CAR-positive viable T cells/kg, about 7.1×10⁶ CAR-positive viable T cells/kg, about 7.2×10⁶ CAR-positive viable T cells/kg, about 7.25×10⁶ CAR-positive viable T cells/kg, about 7.3×10⁶ CAR-positive viable T cells/kg, about 7.4×10⁶ CAR-positive viable T cells/kg, about 7.5×10⁶ CAR-positive viable T cells/kg, about 7.6×10⁶ CAR-positive viable T cells/kg, about 7.7×10⁶ CAR-positive viable T cells/kg, about 7.75×10⁶ CAR-positive viable T cells/kg, about 7.8×10⁶ CAR-positive viable T cells/kg, about 7.9×10⁶ CAR-positive viable T cells/kg, about 8.0×10⁶ CAR-positive viable T cells/kg, about 8.1×10⁶ CAR-positive viable T cells/kg, about 8.2×10⁶ CAR-positive viable T cells/kg, about 8.25×10⁶ CAR-positive viable T cells/kg, about 8.3×10⁶ CAR-positive viable T cells/kg, about 8.4×10⁶ CAR-positive viable T cells/kg, about 8.5×10⁶ CAR-positive viable T cells/kg, about 8.6×10⁶ CAR-positive viable T cells/kg, about 8.7×10⁶ CAR-positive viable T cells/kg, about 8.75×10⁶ CAR-positive viable T cells/kg, about 8.8×10⁶ CAR-positive viable T cells/kg, about 8.9×10⁶ CAR-positive viable T cells/kg, about 9.0×10⁶ CAR-positive viable T cells/kg, about 9.1×10⁶ CAR-positive viable T cells/kg, about 9.2×10⁶ CAR-positive viable T cells/kg, about 9.25×10⁶ CAR-positive viable T cells/kg, about 9.3×10⁶ CAR-positive viable T cells/kg, about 9.4×10⁶ CAR-positive viable T cells/kg, about 9.5×10⁶ CAR-positive viable T cells/kg, about 9.6×10⁶ CAR-positive viable T cells/kg, about 9.7×10⁶ CAR-positive viable T cells/kg, about 9.75×10⁶ CAR-positive viable T cells/kg, about 3.8×10⁶ CAR-positive viable T cells/kg, about 3.9×10⁶ CAR-positive viable T cells/kg, or about 10×10⁶ CAR-positive viable T cells/kg.

### Treatment Indications

As described herein, the methods of the present invention relate to the treatment of a tumor or cancer, such as hematologic malignancies. Hematologic malignancies that may be capable of being treated and/or prevented by the methods of the invention may include, for example, any hematologic tumor or cancer that expresses PD-L1 or that over-expresses PD-L1 as compared to normal cells.

Non-limiting examples of hematologic malignancies treatable by the methods described herein include, for example, lymphoma, multiple myeloma, medullary carcinoma, B cell lymphoma, T cell lymphoma, NK cell lymphoma, large granular lymphocytic lymphoma or leukemia, gamma-delta T cell lymphoma or gamma-delta T cell leukemia, mantle cell lymphoma, myeloma, leukemia, chronic myeloid leukemia, acute myeloid leukemia, chronic lymphocytic leukemia, acute lymphocytic leukemia, hematopoietic neoplasias, thymoma, sarcoma, non-Hodgkin's lymphoma, Hodgkin's lymphoma, malignant lymphoma; Hodgkin's disease; paragranuloma; small lymphocytic malignant lymphoma; diffuse large cell malignant lymphoma; primary mediastinal large B-cell lymphoma; diffuse large B cell lymphoma; follicular malignant lymphoma; mycosis fungoides; other specified non-Hodgkin's lymphomas; malignant histiocytosis; mast cell sarcoma; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia, and all forms of post-transplant lymphomas including post-transplant lymphoproliferative disorder (PTLD).

In certain embodiments, the hematologic malignancy is a lymphoma. In certain embodiments, the hematologic malignancy is a non-Hodgkin lymphoma (NHL). In certain embodiments, the hematologic malignancy is a diffuse large B cell lymphoma (DLBCL). In certain embodiments, the hematologic malignancy is a relapsed/refractory DLBCL.

In some embodiments, the subject may have undergone surgery to remove a large bulk of the tumor, and the methods disclosed herein may be applied before and/or after excision of the bulk of the tumour. In other embodiments, the subject may have been given radiation therapy, chemotherapy or some other non-surgical treatment to control or kill cancerous or malignant cells, and the methods disclosed herein may be applied prior to or subsequent to these therapies. In certain embodiments, the cancer is at an advanced stage.

As discussed above, in treating and/or preventing cancer, the methods of the disclosure may be used to improve the efficacy of a PD-L1 or PD-1 blockade treatment, as described herein. The PD-L1 or PD-1 blockade treatment may involve administering to the subject an inhibitor of PD-1 or PD-L1 selected from, but not limited to, pembrolizumab, nivolumab, pidilizumab, AMP-224, RMP1-4, J43, atezolizumab, avelumab, BMS-936559, durvalumab, tislelizumab, cemiplimab, or a combination thereof.

In some embodiments, the methods described herein may involve administering one or more active agent to “improve the efficacy of the T cell activation therapeutic”, as this expression is described herein. This may involve improving the efficacy of the T cell activation therapeutic in inducing either or both of a cell-mediated immune response or a humoral immune response. This may also involve reducing tumor-induced immune suppression.

As cell mediated immunity involves the participation of various cell types and is mediated by different mechanisms, several methods could be used to demonstrate the induction or improved efficacy of immunity following application of the methods of the invention. These could be broadly classified into detection of: i) specific antigen presenting cells; ii) specific effector cells and their functions and iii) release of soluble mediators such as cytokines.

i) Antigen presenting cells: Dendritic cells and B cells (and to a lesser extent macrophages) are equipped with special immuno-stimulatory receptors that allow for enhanced activation of T cells, and are termed professional antigen presenting cells (APC). These immuno-stimulatory molecules (also called as co-stimulatory molecules) are up-regulated on these cells following infection or vaccination, during the process of antigen presentation to effector cells such as CD4+ and CD8+ cytotoxic T cells. Such co-stimulatory molecules (such as CD80, CD86, MHC class I or MHC class II) can be detected by using flow cytometry with fluorochrome-conjugated antibodies directed against these molecules along with antibodies that specifically identify APC (such as CD11 c for dendritic cells).

ii) Cytotoxic T cells: (also known as Tc, killer T cell, or cytotoxic T-lymphocyte (CTL)) are a sub-group of T cells which induce the death of cells that are infected with viruses (and other pathogens), or expressing tumor antigens. These CTLs directly attack other cells carrying certain foreign or abnormal molecules on their surface. The ability of such cellular cytotoxicity can be detected using in vitro cytolytic assays (chromium release assay). Thus, induction of adaptive cellular immunity can be demonstrated by the presence of such cytotoxic T cells, wherein, when antigen loaded target cells are lysed by specific CTLs that are generated in vivo following vaccination or infection.

Naive cytotoxic T cells are activated when their T cell receptor (TCR) strongly interacts with a peptide-bound MHC class I molecule. This affinity depends on the type and orientation of the antigen/MHC complex, and is what keeps the CTL and infected cell bound together. Once activated the CTL undergoes a process called clonal expansion in which it gains functionality, and divides rapidly, to produce an army of “armed”-effector cells.

Activated CTL will then travel throughout the body in search of cells bearing that unique MHC Class I+peptide. This could be used to identify such CTLs in vitro by using peptide-MHC Class I tetramers in flow cytometric assays.

When exposed to these infected or dysfunctional somatic cells, effector CTL release perform and granulysin: cytotoxins which form pores in the target cell's plasma membrane, allowing ions and water to flow into the infected cell, and causing it to burst or lyse. CTL release granzyme, a serine protease that enters cells via pores to induce apoptosis (cell death). Release of these molecules from CTL can be used as a measure of successful induction of cellular immune response following vaccination. This can be done by enzyme linked immunosorbant assay (ELISA) or enzyme linked immunospot assay (ELISPOT) where CTLs can be quantitatively measured. Since CTLs are also capable of producing important cytokines such as IFN-γ, quantitative measurement of IFN-γ-producing CD8 cells can be achieved by ELISPOT and by flowcytometric measurement of intracellular IFN-γ in these cells.

CD4+“helper” T cells: CD4+ lymphocytes, or helper T cells, are immune response mediators, and play an important role in establishing and maximizing the capabilities of the adaptive immune response. Helper T cells are programmed upon activated by APCs and can direct the type of immune response to eliminate different types of pathogens through secretion of discrete cytokines, for example Th1 helper T cells secrete IFN-gamma, IL-2 and IL-12 and promote the activity of cytotoxic T cells and Th2 helper T cells secrete IL-4, IL-5 and IL-10 and promote the activity of B cells.

Helper T cells express T cell receptors (TCR) that recognize antigen bound to Class II MHC molecules. The activation of a naive helper T cell causes it to release cytokines, which influences the activity of many cell types, including the APC that activated it. The type of activate T helper cell populations can be defined by the pattern of the effector proteins (cytokines) produced. For example, Th1 cells assist the cellular immune response by activation of macrophages and cytotoxic T cells, whereas Th2 cells promote the humoral immune response by stimulation of B cells for conversion into plasma cells and by formation of antibodies. For example, a response regulated by Th1 cells may induce IgG2a and IgG2b in mouse (IgG1 and IgG3 in humans) and favor a cell mediated immune response to an antigen. If the IgG response to an antigen is regulated by Th2 type cells, it may predominantly enhance the production of IgG1 in mouse (IgG2 in humans). The measure of cytokines associated with Th1 or Th2 responses will give a measure of successful vaccination. This can be achieved by specific ELISA designed for Th1-cytokines such as IFN-γ, IL-2, IL-12, TNF-α and others, or Th2-cytokines such as IL-4, IL-5, IL-10 among others.

iii) Measurement of cytokines: released from regional lymph nodes gives a good indication of successful immunization. As a result of antigen presentation and maturation of APC and immune effector cells such as CD4+ and CD8+ T cells, several cytokines are released by lymph node cells. By culturing these LNC in vitro in the presence of antigen, antigen-specific immune response can be detected by measuring release if certain important cytokines such as IFN-γ, IL-2, IL-12, TNF-α and GM-CSF. This could be done by ELISA using culture supernatants and recombinant cytokines as standards.

Successful immunization may be determined in a number of ways known to the skilled person including, but not limited to, hemagglutination inhibition (HAIJ and serum neutralization inhibition assays to detect functional antibodies; challenge studies, in which vaccinated subjects are challenged with the associated pathogen to determine the efficacy of the vaccination; and the use of fluorescence activated cell sorting (FACS) to determine the population of cells that express a specific cell surface marker, e.g., in the identification of activated or memory lymphocytes. A skilled person may also determine if the methods of the invention improved the efficacy of a cell mediated immune response using other known methods. See, for example, Current Protocols in Immunology Coligan et al., ed. (Wiley Interscience, 2007).

In some embodiments, the methods of the invention may also be used to treat cancer by inducing a humoral immune response or by improving the efficacy of the T cell activation therapeutic in inducing a humoral immune response. Such embodiments may have particular application in instances where the T cell activation therapeutic of the invention includes an additional antigen as described herein, other than a survivin antigen. These methods may involve the treatment of cancer by inducing both a cell-mediated immune response and a humoral immune response.

A humoral immune response, as opposed to cell-mediated immunity, is mediated by secreted antibodies which are produced in the cells of the B lymphocyte lineage (B cells). Such secreted antibodies bind to antigens, such as for example those on the surfaces of foreign substances and/or pathogens (e.g., viruses, bacteria, etc.) and flag them for destruction.

Antibodies are the antigen-specific glycoprotein products of a subset of white blood cells called B lymphocytes (B cells). Engagement of antigen with antibody expressed on the surface of B cells can induce an antibody response comprising stimulation of B cells to become activated, to undergo mitosis and to terminally differentiate into plasma cells, which are specialized for synthesis and secretion of antigen-specific antibody.

B cells are the sole producers of antibodies during an immune response and are thus a key element to effective humoral immunity. In addition to producing large amounts of antibodies, B cells also act as antigen-presenting cells and can present antigen to T cells, such as T-helper CD4 or cytotoxic CD8, thus propagating the immune response. B cells, as well as T cells, are part of the adaptive immune response which may assist in T cell activation therapeutic efficacy. During an active immune response, induced either by vaccination or natural infection, antigen-specific B cells are activated and clonally expand. During expansion, B cells evolve to have higher affinity for the epitope. Proliferation of B cells can be induced indirectly by activated T-helper cells, and also directly through stimulation of receptors, such as the toll-like receptors (TLRs).

Antigen presenting cells, such as dendritic cells, B cells and macrophages, are drawn to injection sites and can interact with antigens and adjuvants contained in the T cell activation therapeutic. The adjuvant stimulates the cells to become activated and the antigen provides the blueprint for the target. Different types of adjuvants provide different stimulation signals to cells. For example, polyI:C polynucleotide (a TLR3 agonist) can activate dendritic cells, but not B cells. Adjuvants such as Pam3Cys, Pam2Cys and FSL-1 are especially adept at activating and initiating proliferation of B cells, which is expected to facilitate the production of an antibody response (Moyle et al., Curr Med Chem, 2008; So., J Immunol, 2012).

As used herein, the term “antibody response” refers to an increase in the amount of antigen-specific antibodies in the body of a subject in response to introduction of the antigen into the body of the subject.

One method of evaluating an antibody response is to measure the titers of antibodies reactive with a particular antigen. This may be performed using a variety of methods known in the art such as enzyme-linked immunosorbent assay (ELISA) of antibody-containing substances obtained from animals. For example, the titers of serum antibodies which bind to a particular antigen may be determined in a subject both before and after exposure to the antigen. A statistically significant increase in the titer of antigen-specific antibodies following exposure to the antigen would indicate the subject had mounted an antibody response to the antigen.

Other assays that may be used to detect the presence of an antigen-specific antibody include, without limitation, immunological assays (e.g., radioimmunoassay (RIA)), immunoprecipitation assays, and protein blot (e.g., Western blot) assays; and neutralization assays (e.g., neutralization of viral infectivity in an in vitro or in vivo assay).

The methods of the invention, by improving the efficacy of the T cell activation therapeutic in inducing a humoral immune response, may be capable of treating and/or preventing cancer.

A humoral immune response is the main mechanism for effective infectious disease T cell activation therapeutics. However, a humoral immune response can also be useful for combating cancer. Complementing a cancer T cell activation therapeutic, that is designed to produce a cytotoxic CD8+ T cell response that can recognize and destroy cancer cells, a B cell mediated response may target cancer cells through other mechanisms which may in some instances cooperate with a cytotoxic CD8+ T cell for maximum benefit. Examples of mechanisms of B cell mediated (e.g., humoral immune response mediated) anti-tumor responses include, without limitation: 1) Antibodies produced by B cells that bind to surface antigens found on tumor cells or other cells that influence tumorigenesis. Such antibodies can, for example, induce killing of target cells through antibody-dependent cell-mediated cytotoxicity (ADCC) or complement fixation, potentially resulting in the release of additional antigens that can be recognized by the immune system; 2) Antibodies that bind to receptors on tumor cells to block their stimulation and in effect neutralize their effects; 3) Antibodies that bind to factors released by or associated with tumor or tumor-associated cells to modulate a signaling or cellular pathway that supports cancer; and 4) Antibodies that bind to intracellular targets and mediate anti-tumor activity through a currently unknown mechanism.

The subject to be treated by the methods described herein may be any vertebrate, preferably a mammal, more preferably a human.

### Kits and Reagents

For practicing the methods of the present disclosure, the compositions as described herein may optionally be provided to a user as a kit. For example, a kit of the invention contains one or more reagents for detection of the PD-L1 expression in a biological sample. Such reagents may be necessary to carry out a detection assay such as multiplex immunofluorescence (mIF) assay, an immunohistochemistry (IHC) assay, a fluorescence in situ hybridization (FISH) assay, RNAscope, or flow cytometry. For example, a kit of the invention contains one or more components of the compositions of the invention. The kit can further comprise one or more additional reagents, packaging material, containers for holding the components of the kit, and an instruction set or user manual detailing preferred methods of using the kit components.

In a particular embodiment, the T cell activating therapeutic of the invention (e.g., DPX-Survivac) is supplied as a kit containing two containers. Container 1, for example, may comprise the lyophilized adjuvant system (e.g., lipid vesicle particles), survivin antigens and adjuvant. Container 2, for example, may contain the oil component (Montanide® ISA51 VG) alone. An appropriate volume (0.1 or 0.5 ml) of the reconstituted T cell activating therapeutic may be injected subcutaneously.

In a particular embodiment, the kit may contain an inhibitor of PD-L1 or PD-1. The inhibitor of PD-L1 or PD-1 may be, for example, pembrolizumab, nivolumab, pidilizumab, AMP-224, RMP1-4, J43, atezolizumab, avelumab, BMS-936559, durvalumab, tislelizumab, cemiplimab, or a combination thereof. The inhibitor of PD-L1 or PD-1 may be included in the kit with a third container, or the inhibitor may be included in container 1 or container 2, as described above. In a particular embodiment, the inhibitor of PD-L1 or PD-1 that is included in the kit is pembrolizumab.

In certain embodiments, the kit may additionally contain an active agent. The active agent may be included in the kit with a fourth container, or the agent may be included in container 1, container 2, or container 3, as described above. In a particular embodiment, the active agent that is included in the kit is an alkylating agent, such as for example, cyclophosphamide.

In other embodiments, the kit may additionally contain an additional therapeutic agent. The additional therapeutic agent may be included in the kit with a fifth container, or the agent may be included in container 1, container 2, container 3, or container 4, as described above.

### Exemplary Embodiments

1. A method of treating a hematologic malignancy in a subject in need thereof, said method comprising
   a) detecting the expression of Programmed Death-Ligand 1 (PD-L1) in a biological sample of the subject; and
   b) administering to the subject a therapeutically effective amount of an inhibitor of PD-L1 or Programmed Death 1 (PD-1), and a therapeutically effective amount of a T cell activation therapeutic, wherein PD-L1 expression is detected in the biological sample.
2. The method of embodiment 1, wherein the biological sample is a tumor sample.
3. The method of embodiment 1 or 2, wherein in step b) PD-L1 expression is detected in at least 1% of the cells in the biological sample.
4. The method of any one of embodiments 1-3, wherein in step b) PD-L1 expression is detected in at least 5% of the cells in the biological sample.
5. The method any one of embodiments 1-4, wherein in step b) PD-L1 expression is detected in at least 10% of the cells in the biological sample.
6. The method of any one of embodiments 3-5, wherein the cells are tumor cells, lymphocytes and/or macrophages.
7. The method of any one of embodiments 3-4, wherein the cells are tumor cells.
8. The method of any one of embodiments 3-7, wherein the cells are CD20+ cells.
9. The method of any one of embodiments 1-5, wherein the detection of PD-L1 expression is performed using a multiplex immunofluorescence (mIF) assay, an immunohistochemistry (IHC) assay, a fluorescence in situ hybridization (FISH) assay, RNAscope, or flow cytometry.
10. The method of any one of embodiments 1-6, wherein the method further comprises obtaining a biological sample from the subject prior to the detecting step.
11. The method of any one of embodiments 1-10, wherein the inhibitor of PD-1 or PD-L1 is an antibody.
12. The method of any one of embodiments 1-7, wherein the inhibitor of PD-1 or PD-L1 is pembrolizumab, nivolumab, pidilizumab, AMP-224, RMP1-4, J43, atezolizumab, avelumab, BMS-936559, durvalumab, tislelizumab, cemiplimab, or a combination thereof.
13. The method of any one of embodiments 1-8, wherein the inhibitor of PD-1 is pembrolizumab.
14. The method of any one of embodiments 1-13, wherein a first dose of the inhibitor of PD-1 or PD-L1 is administered to the subject followed by one or more maintenance doses of the inhibitor of PD-1 or PD-L1.
15. The method of any one of embodiments 1-9, wherein the inhibitor of PD-1 or PD-L1 is administered about every 1 to 9 weeks.
16. The method of embodiment 10, wherein the inhibitor of PD-1 or PD-L1 is administered about every 1 to 6 weeks.
17. The method of embodiment 16, wherein the inhibitor of PD-1 or PD-L1 is administered every 2 weeks.
18. The method of embodiment 16, wherein the inhibitor of PD-1 or PD-L1 is administered every 3 weeks.
19. The method of embodiment 16, wherein the inhibitor of PD-1 or PD-L1 is administered every 4 weeks.
20. The method of embodiment 16, wherein the inhibitor of PD-1 or PD-L1 is administered every 6 weeks.
21. The method of any one of embodiments 1-20, wherein the inhibitor of PD-1 or PD-L1 is administered before, after, or concurrently with the T cell activation therapeutic.
22. The method of any one of embodiments 1-11, wherein the first dose of the inhibitor of PD-1 or PD-L1 is administered to the subject on the same day as the first dose of the T cell activation therapeutic.
23. The method of any one of embodiments 1-11, wherein the first dose of the inhibitor of PD-1 or PD-L1 is administered to the subject after the first dose of the T cell activation therapeutic.
24. The method of embodiment 13, wherein the first dose of the inhibitor of PD-1 or PD-L1 is administered to the subject the day after the first dose of the T cell activation therapeutic.
25. The method of any one of embodiments 1-11, wherein the first dose of the inhibitor of PD-1 or PD-L1 is administered to the subject before the first dose of the T cell activation therapeutic.
26. The method of embodiment 25, wherein the first dose of the inhibitor of PD-1 or PD-L1 is administered to the subject the day before the first dose of the T cell activation therapeutic.
27. The method of any one of embodiments 1-26, wherein administration of the inhibitor of PD-1 or PD-L1 continues during the course of administering the T cell activation therapeutic.
28. The method of any one of embodiments 1-14, wherein the inhibitor of PD-1 or PD-L1 is administered at about 50 mg per dose to about 1500 mg per dose.
29. The method of any one of embodiments 1-15, wherein the inhibitor of PD-1 or PD-L1 is administered at about 100 mg per dose.
30. The method of any one of embodiments 1-15, wherein the inhibitor of PD-1 or PD-L1 is administered at about 200 mg per dose.
31. The method of any one of embodiments 1-15, wherein the inhibitor of PD-1 or PD-L1 is administered at about 400 mg per dose.
32. The method of any one of embodiments 1-15, wherein the inhibitor of PD-1 or PD-L1 is administered at about 480 mg per dose.
33. The method of any one of embodiments 1-15, wherein the inhibitor of PD-1 or PD-L1 is administered at about 1200 mg per dose.
34. The method of any one of embodiments 1-15, wherein the inhibitor of PD-1 or PD-L1 is administered in an amount less than 300 mg per dose.
35. The method of any one of embodiments 1-34, wherein the inhibitor of PD-1 or PD-L1 is administered at about 200 mg/day.
36. The method of any one of embodiments 1-35, wherein the inhibitor of PD-1 or PD-L1 is administered by injection to the subject.
37. The method of embodiment 36, wherein the injection is an intravenous, subcutaneous, intertumoral, or intramuscular injection.
38. The method of any one of embodiments 1-37, wherein the T cell activation therapeutic comprises at least one survivin antigen.
39. The method of embodiment 16, wherein the survivin antigen is a peptide antigen or a nucleic acid encoding the peptide antigen.
40. The method of embodiment 16 or 17, wherein the survivin antigen is a peptide antigen comprising an amino acid sequence from the survivin protein (SEQ ID NO: 1) that is capable of eliciting a cytotoxic T-lymphocyte (CTL) response in the subject, or a nucleic acid molecule encoding said peptide antigen.
41. The method of any one of embodiments 16-18, wherein the survivin antigen is a peptide antigen comprising at least one of amino acid sequence FEELTLGEF (SEQ ID NO: 2); FTELTLGEF (SEQ ID NO: 3); LTLGEFLKL (SEQ ID NO: 4); LMLGEFLKL (SEQ ID NO: 5); RISTFKNWPF (SEQ ID NO: 6); RISTFKNWPK (SEQ ID NO: 7); STFKNWPFL (SEQ ID NO: 8); or LPPAWQPFL (SEQ ID NO: 9), or a nucleic acid molecule encoding said peptide antigen.
42. The method of any one of embodiments 16-19, wherein the at least one survivin antigen comprises a mixture of five peptide antigens comprising the amino acid sequence FTELTLGEF (SEQ ID NO: 3); LMLGEFLKL (SEQ ID NO: 5); RISTFKNWPK (SEQ ID NO: 7); STFKNWPFL (SEQ ID NO: 8) or LPPAWQPFL (SEQ ID NO: 9).
43. The method of any one of embodiments 16-20, wherein the at least one survivin antigen is administered at a concentration of about 0.1 mg/ml to about 5 mg/ml for each peptide antigen.
44. The method of embodiment 21, wherein the least one survivin antigen is administered at a concentration of about 1 mg/ml for each peptide antigen.
45. The method of any one of embodiment 21 or embodiment 44, wherein the T cell activation therapeutic is administered at a dose of about 0.01 ml to about 1 ml.
46. The method of embodiment 45, wherein the T cell activation therapeutic is administered at a dose of about 0.25 ml or about 0.5 ml.
47. The method of any one of embodiments 1-46, wherein the T cell activation therapeutic antigen is administered a priming dose of about 0.01 ml to about 1 ml.
48. The method of embodiment 47, wherein the T cell activation therapeutic is administered at a priming dose of about 0.25 ml or about 0.5 ml.
49. The method of any one of embodiments 1-48, wherein the T cell activation therapeutic is administered a booster dose of about 0.01 ml to about 1 ml.
50. The method of embodiment 49, wherein the T cell activation therapeutic is administered at a booster dose of about 0.1 ml.
51. The method of any one of embodiments 1-50, wherein the T cell activation therapeutic is a composition comprising the at least one survivin antigen, lipid vesicle particles, and a carrier comprising a continuous phase of a hydrophobic substance.
52. The method of embodiment 22, wherein the composition further comprises a T-helper epitope.
53. The method of embodiment 23, wherein the T-helper epitope is a peptide comprising the amino acid sequence AQYIKANSKFIGITEL (SEQ ID NO: 10).
54. The method of any one of embodiments 22-24, wherein the composition further comprises an adjuvant.
55. The method of embodiment 25, wherein the adjuvant is a polynucleotide, wherein the polynucleotide is DNA or RNA based.
56. The method of embodiment 25 or 26, wherein the adjuvant is a polyI.C polynucleotide and wherein the polyI.C polynucleotide is DNA or RNA based.
57. The method of any one of embodiments 22-27, wherein the hydrophobic substance is a vegetable oil, nut oil, or mineral oil.
58. The method of any one of embodiments 22-28, wherein the carrier is mineral oil or is a mannide oleate in a mineral oil solution.
59. The method of embodiment 29, wherein the carrier is Montanide® ISA 51.
60. The method of any one of embodiments 1-30, wherein the T cell activation therapeutic is administered by injection to the subject.
61. The method of embodiment 31, wherein the injection is a subcutaneous injection.
62. The method of any one of embodiments 1-61, wherein step b) further comprises administering an effective amount of one or more active agent to the subject.
63. The method of embodiment 32, wherein the active agent is an agent that interferes with DNA replication.
64. The method of embodiment 33, wherein the active agent is capable of selectively targeting rapidly dividing cells of the immune system and causing programmed cell death.
65. The method of any one of embodiments 32-64, wherein the active agent is an alkylating agent.
66. The method of embodiment 65, wherein the alkylating agent is a nitrogen mustard alkylating agent.
67. The method of embodiment 34, wherein the nitrogen mustard alkylating agent is cyclophosphamide.
68. The method of any one of embodiments 32-64, wherein the active agent is at least one of gemcitabine, 5-FU, cisplatin, oxaliplatin, temozolomide, paclitaxel, capecitabine, methotrexate, epirubicin, idarubicin, mitoxantrone, bleomycin, decitabine, or docetaxel.
69. The method of embodiment 32, wherein the active agent is at least one of thalidomide, bortezomib, IL-2, IL-12, IL-15, IFN-gamma, IFN-alpha, TNF-alpha, metformin, or lenalidomide.
70. The method of embodiment 32, wherein the active agent is an inhibitor of at least one of VEGF, a VEGFR, or CD40.
71. The method of any one of embodiments 32-70, wherein the active agent improves the efficacy of the T cell activation therapeutic by directly enhancing the immune response against the antigen, such as by increasing the activity or number of antigen-specific CD8+ T cells.
72. The method of embodiment 71, wherein increasing the activity or number of antigen-specific CD8+ T cells involves an enrichment of antigen-specific CD8+ T cells due to a relative decrease in total CD8+ T cells.
73. The method of any one of embodiments 32-72, wherein the active agent improves the efficacy of the T cell activation therapeutic by reducing the number or activity of suppressive immune cells, for example CD4+FoxP3+ regulatory T cells (Tregs), myeloid-derived suppressor cells (MDSCs), and/or CD19+CD1d+CD5+ B cells (Bregs).
74. The method of any one of embodiments 32-73, wherein the active agent is administered orally to the subject.
75. The method of any one of embodiments 32-36, wherein the active agent is administered by injection to the subject.
76. The method of embodiment 75, wherein the injection is an intravenous, subcutaneous, intertumoral, or intramuscular injection.
77. The method of any one of embodiments 32-76, wherein the effective amount of the active agent is an amount sufficient to provide an immune-modulating effect.
78. The method of any one of embodiments 32-37, wherein the active agent is administered at about 25-300 mg/day, about 50-100 mg/day, or about 100 mg/day.
79. The method of any one of embodiments 32-38, wherein the active agent is administered at about 50 mg per dose.
80. The method of any one of embodiments 32-39, wherein the active agent is administered to the subject at least 1, 2, 3, or 4 times daily.
81. The method of embodiment 40, wherein the active agent is administered twice a day.
82. The method of any one of embodiments 32-81, wherein the active agent is administered before, after, or concurrently with the T cell activation therapeutic.
83. The method of any one of embodiments 32-41, wherein step b) comprises administering a first dose of the active agent to the subject at least two days prior to administering the T cell activation therapeutic.
84. The method of any one of embodiments 32-42, wherein step b) comprises administering a first dose of the active agent to the subject about one week prior to administering the T cell activation therapeutic.
85. The method of any one of embodiments 32-84, wherein step b) comprises administering to the subject a first dose of the active agent, followed by one or more maintenance doses of the active agent.
86. The method of any one of embodiments 32-43, wherein step b) comprises administering the active agent to the subject twice daily for a period of about one week.
87. The method of any one of embodiments 32-44, wherein step b) comprises administering the active agent to the subject in a low dose metronomic regimen.
88. The method of embodiment 45, wherein the metronomic regimen comprises administering the active agent to the subject daily for a period of about one week every second week.
89. The method of embodiment 45 or 46, wherein the metronomic regimen comprises administering the active agent for a two-week cycle, wherein the active agent is administered to the subject during the first week of the cycle, wherein the active agent is not administered to the subject during the second week of the cycle, and wherein the metronomic regimen comprises at least two cycles.
90. The method of any one of embodiments 1-47, wherein step b) comprises administering the T cell activation therapeutic to the subject about once every three weeks.
91. The method of any one of embodiments 1-47, wherein step b) comprises administering the T cell activation therapeutic to the subject about once every eight weeks.
92. The method of any one of embodiments 1-47, wherein step b) comprises administering first two doses of the T cell activation therapeutic to the subject about three weeks apart, and then administering the T cell activation therapeutic to the subject about once every eight weeks.
93. The method of any one of embodiments 32-47, wherein step b) comprises administering the active agent to the subject beginning about one week before administering a first dose of the T cell activation therapeutic, and administering the T cell activation therapeutic to the subject about once every three weeks.
94. The method of any one of embodiments 32-47, wherein step b) comprises administering the active agent to the subject beginning about one week before administering a first dose of the T cell activation therapeutic, administering a second dose of the T cell activation therapeutic to the subject about three weeks after the first dose, and then administering the T cell activation therapeutic to the subject about once every eight weeks.
95. The method of any one of embodiments 1-48, wherein step b) further comprises administering at least one additional therapeutic agent.
96. The method of embodiment 95, wherein the at least one additional therapeutic agent is one or more of Rituximab, obinutuzumab, Cyclophosphamide, Mitoxantrone, Vincristine, Prednisone, etoposide, Methotrexate/Ifosfamide and Cytarabine, Dexamethasone, Cisplatin, Gemcitabine, Brentuximab vedotin, Bendamustine, liposomal Doxorubicin, Lenalidomide, ibrutinib, selinixor, Polatuzumab vedotin, Pixantrone, CAR-T cells (e.g. Yescarta, Kymriah), mozunetuzumab, bispecific antibodies, thalidomide, Autologous Stem Cell Transplant, or tafasitamab.
97. The method of any one of embodiments 95-96, wherein the at least one additional therapeutic agent is administered concurrently or sequentially with the inhibitor of PD-1 or PD-L1, T cell activation therapeutic and/or active agent.
98. The method of any one of embodiments 1-97, wherein the hematologic malignancy is non-Hodgkin lymphoma (NHL).
99. The method of any one of embodiments 1-49, wherein the hematologic malignancy is diffuse large B cell lymphoma (DLBCL).
100. The method of embodiment 50, wherein the DLBCL is a relapsed/refractory DLBCL.
101. The method of any one of embodiments 1-51, wherein the subject is a human.
102. A method of identifying a subject who is likely to benefit from a combination treatment comprising an inhibitor of PD-L1 or Programmed Death 1 (PD-1) and a T cell activation therapeutic, wherein the subject has a hematologic malignancy, comprising
   a) detecting the expression of Programmed Death-Ligand 1 (PD-L1) in a biological sample of the subject; and
   b) identifying the subject as likely to benefit from the combination treatment comprising an inhibitor of PD-L1 or PD-1 and a T cell activation therapeutic, wherein PD-L1 expression is detected in the biological sample.
103. The method of embodiment 102, wherein the biological sample is a tumor sample.
104. The method of embodiment 102 or 103, wherein in step b) PD-L1 expression is detected in at least 1% of the cells in the biological sample.
105. The method of any one of embodiments 102-104, wherein in step b) PD-L1 expression is detected in at least 5% of the cells in the biological sample.
106. The method any one of embodiments 102-105, wherein in step b) PD-L1 expression is detected in at least 10% of the cells in the biological sample.
107. The method of any one of embodiments 104-106, wherein the cells are tumor cells, lymphocytes and/or macrophages.
108. The method of any one of embodiments 104-107, wherein the cells are tumor cells.
109. The method of any one of embodiments 104-108, wherein the cells are CD20+ cells.
110. The method of any one of embodiments 102-109, wherein the detection of PD-L1 expression is performed using a multiplex immunofluorescence (mIF) assay, an immunohistochemistry (IHC) assay, a fluorescence in situ hybridization (FISH) assay, RNAscope, or flow cytometry.
111. The method of any one of embodiments 102-110, wherein the method further comprises obtaining a biological sample from the subject prior to the detecting step.
112. The method of any one of embodiments 102-111, wherein the inhibitor of PD-1 or PD-L1 is an antibody.
113. The method of any one of embodiments 102-112, wherein the inhibitor of PD-1 or PD-L1 is pembrolizumab, nivolumab, pidilizumab, AMP-224, RMP1-4, J43, atezolizumab, avelumab, BMS-936559, durvalumab, tislelizumab, cemiplimab, or a combination thereof.
114. The method of any one of embodiments 102-113, wherein the inhibitor of PD-1 is pembrolizumab.
115. The method of any one of embodiments 102-114, wherein a first dose of the inhibitor of PD-1 or PD-L1 is administered to the subject followed by one or more maintenance doses of the inhibitor of PD-1 or PD-L1.
116. The method of any one of embodiments 102-115, wherein the inhibitor of PD-1 or PD-L1 is administered about every 1 to 9 weeks.
117. The method of embodiment 116, wherein the inhibitor of PD-1 or PD-L1 is administered about every 1 to 6 weeks.
118. The method of embodiment 117, wherein the inhibitor of PD-1 or PD-L1 is administered every 2 weeks.
119. The method of embodiment 117, wherein the inhibitor of PD-1 or PD-L1 is administered every 3 weeks.
120. The method of embodiment 117, wherein the inhibitor of PD-1 or PD-L1 is administered every 4 weeks.
121. The method of embodiment 117, wherein the inhibitor of PD-1 or PD-L1 is administered every 6 weeks.
122. The method of any one of embodiments 102-121, wherein the inhibitor of PD-1 or PD-L1 is administered before, after, or concurrently with the T cell activation therapeutic.
123. The method of any one of embodiments 102-122, wherein the first dose of the inhibitor of PD-1 or PD-L1 is administered to the subject on the same day as the first dose of the T cell activation therapeutic.
124. The method of any one of embodiments 102-122, wherein the first dose of the inhibitor of PD-1 or PD-L1 is administered to the subject after the first dose of the T cell activation therapeutic.
125. The method of embodiment 124, wherein the first dose of the inhibitor of PD-1 or PD-L1 is administered to the subject the day after the first dose of the T cell activation therapeutic.
126. The method of any one of embodiments 102-122, wherein the first dose of the inhibitor of PD-1 or PD-L1 is administered to the subject before the first dose of the T cell activation therapeutic.
127. The method of embodiment 126, wherein the first dose of the inhibitor of PD-1 or PD-L1 is administered to the subject the day before the first dose of the T cell activation therapeutic.
128. The method of any one of embodiments 102-127, wherein administration of the inhibitor of PD-1 or PD-L1 continues during the course of administering the T cell activation therapeutic.
129. The method of any one of embodiments 102-128, wherein the inhibitor of PD-1 or PD-L1 is administered at about 50 mg per dose to about 1500 mg per dose.
130. The method of any one of embodiments 102-129, wherein the inhibitor of PD-1 or PD-L1 is administered at about 100 mg per dose.
131. The method of any one of embodiments 102-129, wherein the inhibitor of PD-1 or PD-L1 is administered at about 200 mg per dose.
132. The method of any one of embodiments 102-129, wherein the inhibitor of PD-1 or PD-L1 is administered at about 400 mg per dose.
133. The method of any one of embodiments 102-129, wherein the inhibitor of PD-1 or PD-L1 is administered at about 480 mg per dose.
134. The method of any one of embodiments 102-129, wherein the inhibitor of PD-1 or PD-L1 is administered at about 1200 mg per dose.
135. The method of any one of embodiments 102-129, wherein the inhibitor of PD-1 or PD-L1 is administered in an amount less than 300 mg per dose.
136. The method of any one of embodiments 102-128, wherein the inhibitor of PD-1 or PD-L1 is administered at about 200 mg/day.
137. The method of any one of embodiments 102-136, wherein the inhibitor of PD-1 or PD-L1 is administered by injection to the subject.
138. The method of embodiment 137, wherein the injection is an intravenous, subcutaneous, intertumoral, or intramuscular injection.
139. The method of any one of embodiments 102-138, wherein the T cell activation therapeutic comprises at least one survivin antigen.
140. The method of embodiment 139, wherein the survivin antigen is a peptide antigen or a nucleic acid encoding the peptide antigen.
141. The method of embodiment 139 or 140, wherein the survivin antigen is a peptide antigen comprising an amino acid sequence from the survivin protein (SEQ ID NO: 1) that is capable of eliciting a cytotoxic T-lymphocyte (CTL) response in the subject, or a nucleic acid molecule encoding said peptide antigen.
142. The method of any one of embodiments 139-141, wherein the survivin antigen is a peptide antigen comprising at least one of amino acid sequence FEELTLGEF (SEQ ID NO: 2); FTELTLGEF (SEQ ID NO: 3); LTLGEFLKL (SEQ ID NO: 4); LMLGEFLKL (SEQ ID NO: 5); RISTFKNWPF (SEQ ID NO: 6); RISTFKNWPK (SEQ ID NO: 7); STFKNWPFL (SEQ ID NO: 8); or LPPAWQPFL (SEQ ID NO: 9), or a nucleic acid molecule encoding said peptide antigen.
143. The method of any one of embodiments 139-142, wherein the at least one survivin antigen comprises a mixture of five peptide antigens comprising the amino acid sequence FTELTLGEF (SEQ ID NO: 3); LMLGEFLKL (SEQ ID NO: 5); RISTFKNWPK (SEQ ID NO: 7); STFKNWPFL (SEQ ID NO: 8) or LPPAWQPFL (SEQ ID NO: 9).
144. The method of any one of embodiments 139-143, wherein the at least one survivin antigen is administered at a concentration of about 0.1 mg/ml to about 5 mg/ml for each peptide antigen.
145. The method of embodiment 144, wherein the least one survivin antigen is administered at a concentration of about 1 mg/ml for each peptide antigen.
146. The method of any one of embodiment 144 or embodiment 145, wherein the T cell activation therapeutic is administered at a dose of about 0.01 ml to about 1 ml.
147. The method of embodiment 146, wherein the T cell activation therapeutic is administered at a dose of about 0.25 ml or about 0.5 ml.
148. The method of any one of embodiments 102-147, wherein the T cell activation therapeutic antigen is administered a priming dose of about 0.01 ml to about 1 ml.
149. The method of embodiment 148, wherein the T cell activation therapeutic is administered at a priming dose of about 0.25 ml or about 0.5 ml.
150. The method of any one of embodiments 102-149, wherein the T cell activation therapeutic is administered a booster dose of about 0.01 ml to about 1 ml.
151. The method of embodiment 150, wherein the T cell activation therapeutic is administered at a booster dose of about 0.1 ml.
152. The method of any one of embodiments 102-151, wherein the T cell activation therapeutic is a composition comprising the at least one survivin antigen, lipid vesicle particles, and a carrier comprising a continuous phase of a hydrophobic substance.
153. The method of embodiment 152, wherein the composition further comprises a T-helper epitope.
154. The method of embodiment 153, wherein the T-helper epitope is a peptide comprising the amino acid sequence AQYIKANSKFIGITEL (SEQ ID NO: 10).
155. The method of any one of embodiments 152-154, wherein the composition further comprises an adjuvant.
156. The method of embodiment 155, wherein the adjuvant is a polynucleotide, wherein the polynucleotide is DNA or RNA based.
157. The method of embodiment 155 or 156, wherein the adjuvant is a polyI.C polynucleotide and wherein the polyI.C polynucleotide is DNA or RNA based.
158. The method of any one of embodiments 152-157, wherein the hydrophobic substance is a vegetable oil, nut oil, or mineral oil.
159. The method of any one of embodiments 152-158, wherein the carrier is mineral oil or is a mannide oleate in a mineral oil solution.
160. The method of embodiment 159, wherein the carrier is Montanide® ISA 51.
161. The method of any one of embodiments 102-160, wherein the T cell activation therapeutic is administered by injection to the subject.
162. The method of embodiment 161, wherein the injection is a subcutaneous injection.
163. The method of any one of embodiments 102-162, wherein the combination treatment further comprises one or more active agent.
164. The method of embodiment 163, wherein the active agent is an agent that interferes with DNA replication.
165. The method of embodiment 164, wherein the active agent is capable of selectively targeting rapidly dividing cells of the immune system and causing programmed cell death.
166. The method of any one of embodiments 163-165, wherein the active agent is an alkylating agent.
167. The method of embodiment 166, wherein the alkylating agent is a nitrogen mustard alkylating agent.
168. The method of embodiment 167, wherein the nitrogen mustard alkylating agent is cyclophosphamide.
169. The method of any one of embodiments 163-165, wherein the active agent is at least one of gemcitabine, 5-FU, cisplatin, oxaliplatin, temozolomide, paclitaxel, capecitabine, methotrexate, epirubicin, idarubicin, mitoxantrone, bleomycin, decitabine, or docetaxel.
170. The method of embodiment 163, wherein the active agent is at least one of thalidomide, bortezomib, IL-2, IL-12, IL-15, IFN-gamma, IFN-alpha, TNF-alpha, metformin, or lenalidomide.
171. The method of embodiment 163, wherein the active agent is an inhibitor of at least one of VEGF, a VEGFR, or CD40.
172. The method of any one of embodiments 163-171, wherein the active agent improves the efficacy of the T cell activation therapeutic by directly enhancing the immune response against the antigen, such as by increasing the activity or number of antigen-specific CD8+ T cells.
173. The method of embodiment 172, wherein increasing the activity or number of antigen-specific CD8+ T cells involves an enrichment of antigen-specific CD8+ T cells due to a relative decrease in total CD8+ T cells.
174. The method of any one of embodiments 163-173, wherein the active agent improves the efficacy of the T cell activation therapeutic by reducing the number or activity of suppressive immune cells, for example CD4+FoxP3+ regulatory T cells (Tregs), myeloid-derived suppressor cells (MDSCs), and/or CD19+CD1d+CD5+ B cells (Bregs).
175. The method of any one of embodiments 163-174, wherein the active agent is administered orally to the subject.
176. The method of any one of embodiments 163-174, wherein the active agent is administered by injection to the subject.
177. The method of embodiment 176, wherein the injection is an intravenous, subcutaneous, intertumoral, or intramuscular injection.
178. The method of any one of embodiments 163-177, wherein the active agent is administered at an amount sufficient to provide an immune-modulating effect.
179. The method of any one of embodiments 163-178, wherein the active agent is administered at about 25-300 mg/day, about 50-100 mg/day, or about 100 mg/day.
180. The method of any one of embodiments 163-179, wherein the active agent is administered at about 50 mg per dose.
181. The method of any one of embodiments 163-180, wherein the active agent is administered to the subject at least 1, 2, 3, or 4 times daily.
182. The method of embodiment 181, wherein the active agent is administered twice a day.
183. The method of any one of embodiments 163-182, wherein the active agent is administered before, after, or concurrently with the T cell activation therapeutic.
184. The method of any one of embodiments 163-183, wherein the combination treatment comprises administering a first dose of the active agent to the subject at least two days prior to administering the T cell activation therapeutic.
185. The method of any one of embodiments 163-184, wherein the combination treatment comprises administering a first dose of the active agent to the subject about one week prior to administering the T cell activation therapeutic.
186. The method of any one of embodiments 163-185, wherein the combination treatment comprises administering to the subject a first dose of the active agent, followed by one or more maintenance doses of the active agent.
187. The method of any one of embodiments 163-186, wherein the combination treatment comprises administering the active agent to the subject twice daily for a period of about one week.
188. The method of any one of embodiments 163-187, wherein the combination treatment comprises administering the active agent to the subject in a low dose metronomic regimen.
189. The method of embodiment 188, wherein the metronomic regimen comprises administering the active agent to the subject daily for a period of about one week every second week.
190. The method of embodiment 188 or 189, wherein the metronomic regimen comprises administering the active agent for a two-week cycle, wherein the active agent is administered to the subject during the first week of the cycle, wherein the active agent is not administered to the subject during the second week of the cycle, and wherein the metronomic regimen comprises at least two cycles.
191. The method of any one of embodiments 102-190, wherein the combination treatment comprises administering the T cell activation therapeutic to the subject about once every three weeks.
192. The method of any one of embodiments 102-190, wherein the combination treatment comprises administering the T cell activation therapeutic to the subject about once every eight weeks.
193. The method of any one of embodiments 102-190, wherein the combination treatment comprises administering first two doses of the T cell activation therapeutic to the subject about three weeks apart, and then administering the T cell activation therapeutic to the subject about once every eight weeks.
194. The method of any one of embodiments 163-190, wherein the combination treatment comprises administering the active agent to the subject beginning about one week before administering a first dose of the T cell activation therapeutic, and administering the T cell activation therapeutic to the subject about once every three weeks.
195. The method of any one of embodiments 163-190, wherein the combination treatment comprises administering the active agent to the subject beginning about one week before administering a first dose of the T cell activation therapeutic, administering a second dose of the T cell activation therapeutic to the subject about three weeks after the first dose, and then administering the T cell activation therapeutic to the subject about once every eight weeks.
196. The method of any one of embodiments 102-195, wherein the combination treatment further comprises at least one additional therapeutic agent.
197. The method of embodiment 196, wherein the at least one additional therapeutic agent is one or more of Rituximab, obinutuzumab, Cyclophosphamide, Mitoxantrone, Vincristine, Prednisone, etoposide, Methotrexate/Ifosfamide and Cytarabine, Dexamethasone, Cisplatin, Gemcitabine, Brentuximab vedotin, Bendamustine, liposomal Doxorubicin, Lenalidomide, ibrutinib, selinixor, Polatuzumab vedotin, Pixantrone, CAR-T cells (e.g. Yescarta, Kymriah), mozunetuzumab, bispecific antibodies, thalidomide, Autologous Stem Cell Transplant, or tafasitamab.
198. The method of any one of embodiments 196-197, wherein the at least one additional therapeutic agent is administered concurrently or sequentially with the inhibitor of PD-1 or PD-L1, T cell activation therapeutic and/or active agent.
199. The method of any one of embodiments 102-198, wherein the hematologic malignancy is non-Hodgkin lymphoma (NHL).
200. The method of any one of embodiments 102-199, wherein the hematologic malignancy is diffuse large B cell lymphoma (DLBCL).
201. The method of embodiment 200, wherein the DLBCL is a relapsed/refractory DLBCL.
202. The method of any one of embodiments 102-201, wherein the subject is a human.

### EXAMPLES

The following examples are provided to further describe some of the embodiments disclosed herein. The examples are intended to illustrate, not to limit, the disclosed embodiments.

### Example 1. Baseline PD-L1 Expression is Associated with Clinical Response in Patients with Recurrent/Refractory (r/r) DLBCL Treated with DPX-Survivac, Low-Dose Cyclophosphamide (CPA) and Pembrolizumab

DPX-Survivac is a unique T cell activation therapy that targets survivin-expressing tumor cells and has shown anti-tumor activity in clinical trials. This trial is evaluating a novel immunotherapy combination with DPX-Survivac, intermittent low dose cyclophosphamide (CPA) and pembrolizumab.

“SPiReL” is a Phase 2 non-randomized, open label, efficacy and safety study of a novel immunotherapy combination with DPX-Survivac (a unique T cell activation therapy that targets survivin-expressing tumor cells^{3,4}), intermittent low dose CPA and pembrolizumab, treatment regimen as described in FIG. 1. Pembrolizumab⁵ a potent humanized mAb with high specificity of binding to PD-1. Cyclophosphamide (CPA) is a potential inhibitor of regulatory T cells pathway⁶.

Subjects with r/r DLBCL and survivin expression are eligible for participation. This study was approved by the Ontario Cancer Research Ethics Board (OCREB), approval number 0981. The ClinicalTrials.gov Registration #is NTC03349450.

### Trial Endpoints

The primary endpoint of this Phase 2 study is to document clinical activity of this treatment approach. Secondary and exploratory endpoints were aimed at documenting safety and to understand the biologic and immune effects of this treatment and to identify biomarkers that would identify patients more likely to respond clinically to this novel immune therapy.

### Trial Design

Participants with r/r DLBCL, with Eastern Cooperative Oncology Group (ECOG) 0-1 and confirmed survivin expression are eligible. Participants must also be ineligible for curative therapy.

Study treatment includes administering two doses of 0.5 mL of DPX-Survivac, subcutaneously, 3 weeks apart followed by up to six 0.1 mL doses every 8 weeks. Intermittent low dose CPA is taken orally at 50 mg twice daily for 7 days followed by 7 days off. Pembrolizumab 200 mg IV is administered every 3 weeks. The treatment regimen is described in FIG. 1.

Study participants continue trial participation for up to one year or until disease progression, whichever occurs first.

Participants are considered evaluable when they have received 3 doses of DPX-Survivac, 4 infusions of pembrolizumab and have had an on-treatment CT scan to assess response.

### Results:

The SPiReL trial is being conducted at 6 Canadian centers. Recruitment began with a goal of 25 evaluable participants. Results presented include data from 40 screened participants, 22 of whom have been enrolled, 11 of which are evaluable.

ORR is assessed by modified Cheson criteria. For translational analyses, baseline and on-treatment PBMCs, along with tumor biopsy samples are collected from each subject. Survivin-specific systemic T cell responses are assessed using IFNγ-ELISpot assay and tumor immune-infiltrate profile by multiplex-immunohistochemistry.

In this Phase 2 study, PD-L1 expression was assessed using multiplex immunochemistry (mIHC) and multiplex-immunofluorescence analyses (mIF) as described in Table 2.

Alternatively, a qualitative immunohistochemical (IHC) assay using monoclonal mouse anti-PD-L1 antibody, Clone 22C3 (currently approved assay with Keytruda) may also be used (see Table 2). The assay detects PD-L1 protein in formalin-fixed, paraffin-embedded (FFPE) tumor tissues using EnVision FLEX visualization system on Autostainer Link 48.

TABLE 2
PD-L1 detection assays

| Test | Method | Antibody | Scoring criteria |
|---|---|---|---|
| PD-L1 | Standard | Dako | Tumor Proportion Score |
| by IHC- | IHC | Clone#22C3 | (TPS)-NSCLC |
| Keytruda | | | % viable tumor cells |
| | | | showing partial or |
| | | | complete membrane |
| | | | staining at any intensity. |
| | | | The specimen should |
| | | | be considered to have |
| | | | PD-L1 expression if |
| | | | TPS ≥1% and high P- |
| | | | DL1 expression |
| | | | if TPS ≥50%. |
| | | | Combined Positive |
| | | | Score (CPS)-Other |
| | | | Indications- |
| | | | number of PD-L1 |
| | | | staining cells |
| | | | (tumor cells, |
| | | | lymphocytes, |
| | | | macrophages) |
| | | | divided by the |
| | | | total number of viable |
| | | | tumor cells, multiplied |
| | | | by 100. |
| | | | The specimen should |
| | | | be considered to have |
| | | | PD-L1 expression |
| | | | if CPS ≥1. |
| PD-L1 | multiplex- | CST | Multiplex assay used to |
| by | Immuno- | Clone#E1L3N | evaluate expression of |
| mIF | fluorescence | | multiple tissue |
| | (mIF)-Akoya | | biomarkers |
| | OPAL | | on a single tissue |
| | Human Panel | | section using different |
| | Assay | | fluorescently labelled |
| | | | antibodies. |
| | | | For PD-L1 expression, |
| | | | available data include: |
| | | | Membrane PD-L1 |
| | | | expression in all cells |
| | | | Membrane PD-L1 |
| | | | expression in CD20+ |
| | | | (tumor cells) |
| | | | All data is available |
| | | | for tumor region, non- |
| | | | tumor region, and |
| | | | total tissue section area |
| | | | Scored as either: |
| | | | Mean fluorescence |
| | | | intensity (MFI) |
| | | | Expression thresholding |
| | | | (0 to 3+) |
| | | | H-Score |
| | | | % positive cells |
| | | | (any intensity) |
| | | | For analyses included |
| | | | herein, % Positive |
| | | | Cells is used which |
| | | | demonstrates Membrane |
| | | | PD-L1 expression (any |
| | | | intensity) in all cells |
| | | | and for the total tissue |
| | | | section area |

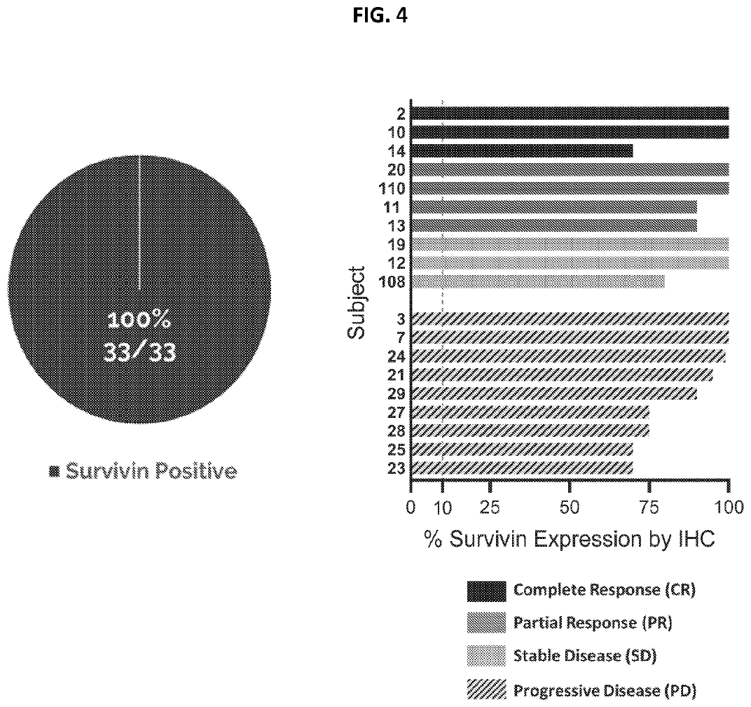

FIG. 4 demonstrates survivin expression in the study population. All 33 subjects in screening who had samples centrally assessed were positive for survivin expression (using a cut-off of expression of >10% by IHC). Enrolled subjects all demonstrate survivin expression >70%, and at this time, no trend is observed between response and survivin expression in this population.

For time on treatment analyses, the overall response rate (ORR) of the subjects included in the full analyses set (N=23) was 30.4% and disease control rate (DCR) was 52.2% (see FIG. 10A). For best overall response analyses, Per Protocol (PP) subjects (N=14) had an ORR of 50% and a DCR of 78.6%, and PD-L1 positive subjects (N=7) had an ORR of 85.7% and a DCR of 85.7% (see FIG. 10B).

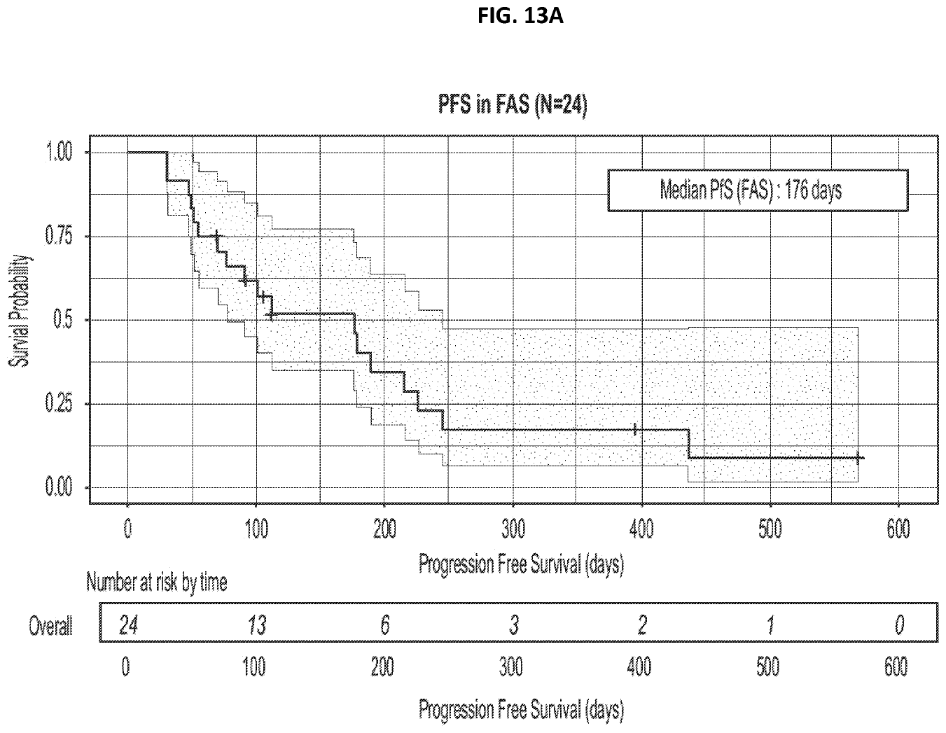

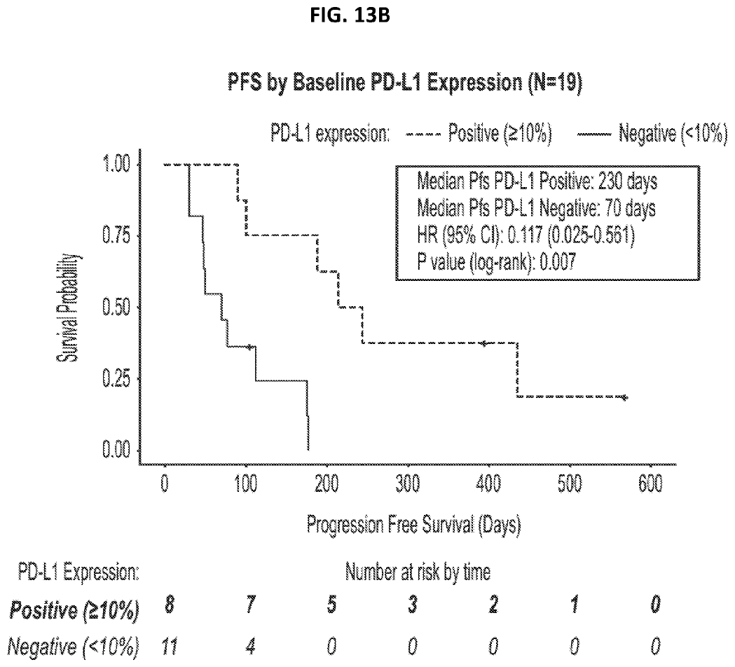

FIG. 13A-13B show graphical representations of progression free survival (PFS) data indicating that the median PFS was significantly higher in the PD-L1+ population, in support of baseline PD-L1 as a biomarker.

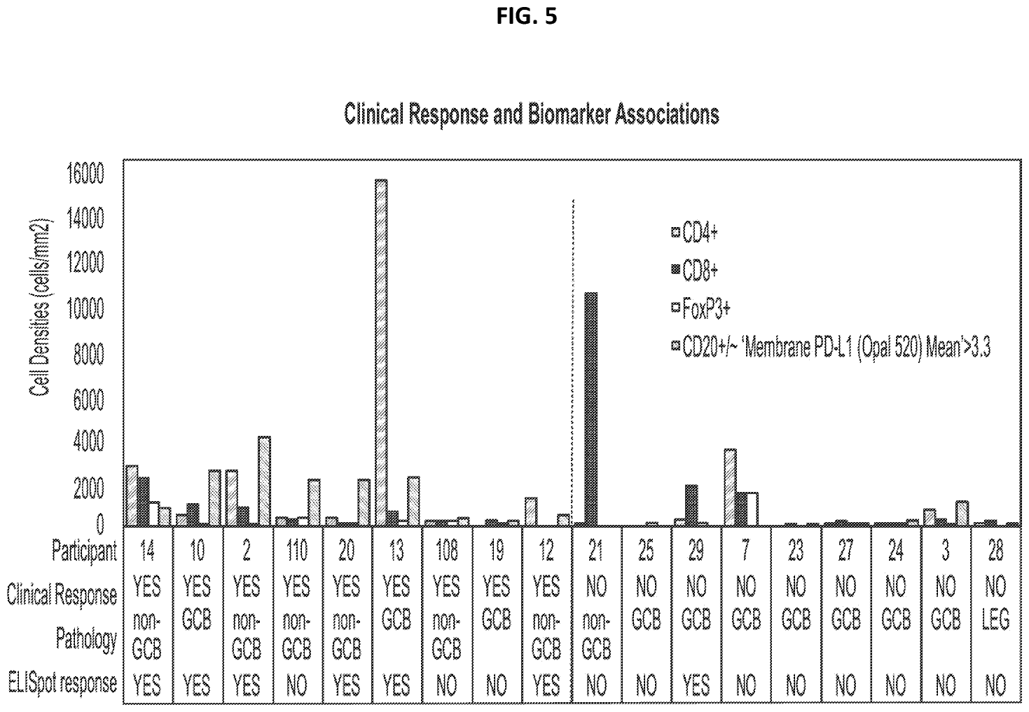

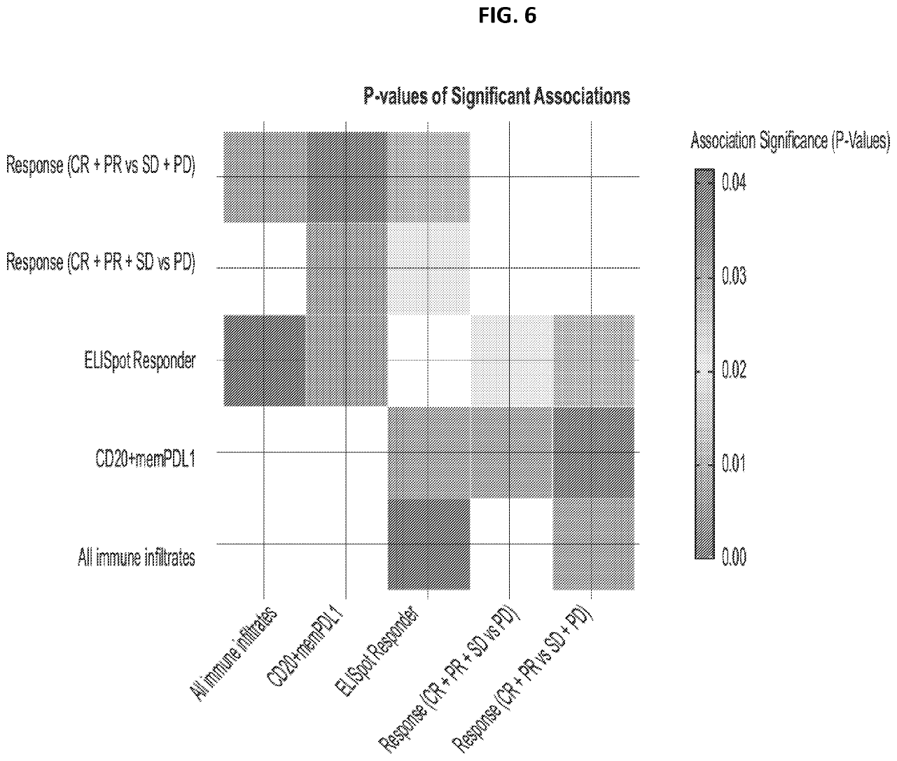

FIG. 5 demonstrates the baseline expression of different tumor immune infiltrates (CD8+, CD4+, PD-L1+, FoxP3+ cells) categorized based on their clinical response, subtypes and ELISpot response status.

There is a statistically significant correlation documented between baseline PD-L1 expression assessed by mIHC and clinical response, as demonstrated by FIG. 8. All analyzed subjects who achieved a CR or PR on study expressed PD-L1 (using a cut-off ≥10% by mIHC analyses), making PD-L1 a possible predictor of response using this combination immunotherapy.

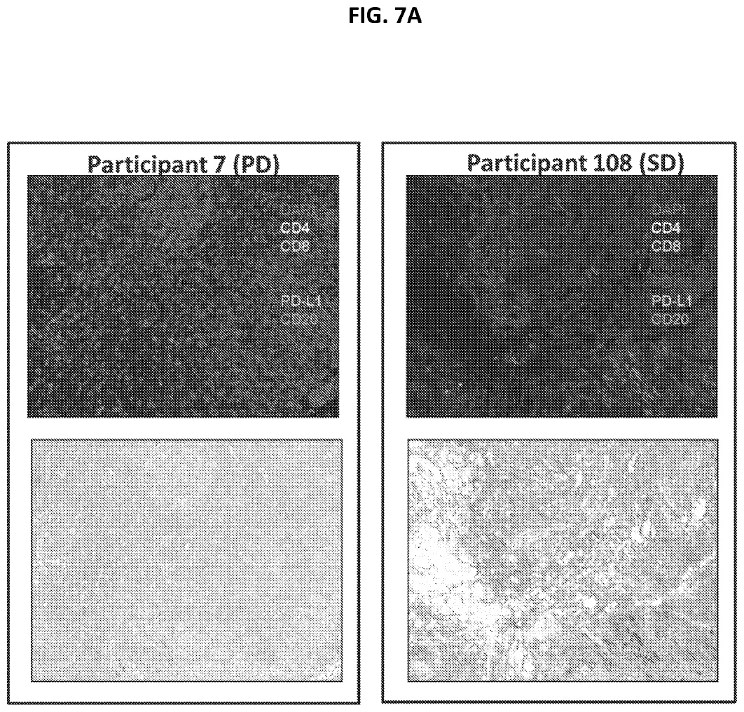

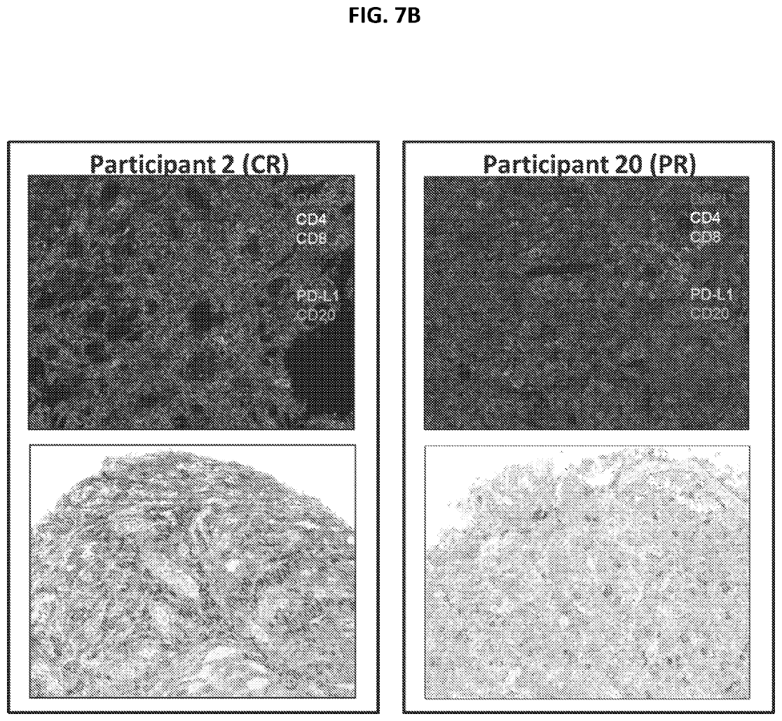

Examples of PD-L1 expression using spectral analysis and simulated IHC analysis in subjects with various clinical responses are shown in FIGS. 7A-7B.

FIG. 9A demonstrates a statistically significant increase in survivin-specific T cell responses when comparing the baseline response with the best on-treatment response. As highlighted in the panel B, DPX-Survivac induced survivin-specific T cell responses are observed in 100% of subjects with a complete response and 75% of subjects with a partial response, supporting the mechanism of action of DPX-Survivac.

DPX-Survivac-induced T cell responses were observed in 8/19 subjects (42.1%) including 6 subjects with clinical response (PR, CR), one SD and one PD. Multiplex-immunohistochemistry analyses demonstrated baseline tumor PD-L1 expression in 6/7 subjects with a clinical response (85.7%, p<0.05). Similarly, subjects with higher baseline CD4+ and CD8+ T cell infiltration demonstrated a trend towards clinical response (FIG. 3).

### CONCLUSION

DPX-Survivac, intermittent low-dose CPA and pembrolizumab is generally well tolerated and can induce clinical responses in subjects with r/r DLBCL (7/11, 63.6% of evaluable subjects), including subjects with both non-GCB and GCB subtypes. Pre-treatment biopsies of clinical responders were characterized by higher baseline tumor PD-L1 expression and CD4 and CD8 infiltration. Extending this exploratory data in a larger cohort may define a r/r DLBCL patient population with a higher likelihood to respond to this novel combination immunotherapy.

The results described above suggest that combination immunotherapy with DPX-Survivac, pembrolizumab and intermittent, low dose cyclophosphamide can induce clinical response and disease control in patients with r/r DLBCL. Clinical responses are associated with higher baseline expression of PD-L1 and DPX-Survivac induced survivin-specific T cell response. Baseline % PD-L1 expression in biological samples may identify patients most likely to respond to this combination immunotherapy. The efficacy of anti-PD-1 of anti-PD-L1 as monotherapy in the treatment of r/r DLBCL has been very limited. The new combination of DPX-Survivac/CPA and pembrolizumab provides clinical responses and disease control rate that were not observed with monotherapy with anti-PD-1 and anti-PD-L1. Additionally, the results show that PD-L1 expression could be use as a predictive biomarker of response in patients with r/r DLBCL. This predictive biomarker can be used to selectively treat patients who can mostly benefit from the treatment. It will provide a more tailored treatment option for the patients.

The association between PD-L1 expression on B lymphoma cells and induction of survivin-specific T cell responses identifies a novel and important interactive immune mechanism of action.

### REFERENCES

1. Crump M, Neelapu S S, Farooq U, et al. Outcomes in refractory diffuse large B-cell lymphoma: results from the international SCHOLAR-1 study. Blood. 2017; 130(16):1800-1808.
2. Neelapu S S, Locke F L, Go W Y. CAR T-Cell Therapy in Large B-Cell Lymphoma. N Engl J Med. 2018; 378(11):1065.
3. Berinstein N L, Karkada M, Oza A M, et al. Survivin-targeted immunotherapy drives robust polyfunctional T cell generation and differentiation in advanced ovarian cancer patients. Oncoimmunology. 2015; 4(8):e1026529.
4. Weir G M, Hrytsenko O, Quinton T, Berinstein N L, Stanford M M, Mansour M. Anti-PD-1 increases the clonality and activity of tumor infiltrating antigen specific T cells induced by a potent immune therapy consisting of vaccine and metronomic cyclophosphamide. J Immunother Cancer. 2016; 4:68.
5. Matsuki E, Younes A. Checkpoint Inhibitors and Other Immune Therapies for Hodgkin and Non-Hodgkin Lymphoma. Curr Treat Options Oncol. 2016; 17(6):31.
6. Sistigu A, Viaud S, Chaput N, Bracci L, Proietti E, Zitvogel L. Immunomodulatory effects of cyclophosphamide and implementations for vaccine design. Semin Immunopathol. 2011; 33(4):369-383.
7. Lee C W, Ren Y J, Marella M, Wang M, Hartke J, Couto S S. Multiplex immunofluorescence staining and image analysis assay for diffuse large B cell lymphoma. J Immunol Methods. 2020 March; 478:112714. doi: 10.1016/j.jim.2019.112714.
8. Huang S, Nong L, Liang L, et al. Comparison of PD-L1 detection assays and corresponding significance in evaluation of diffuse large B-cell lymphoma. Cancer Med. 2019; 8(8):3831-3845. doi:10.1002/cam4.2316
9. Davis A A, Patel V G. The role of PD-L1 expression as a predictive biomarker: an analysis of all US Food and Drug Administration (FDA) approvals of immune checkpoint inhibitors. J. Immunotherapy Cancer 7, 278 (2019). doi.org/10.1186/s40425-019-0768-9
10. Ansell S M, Minnema M C, Johnson P, et al. Nivolumab for Relapsed/Refractory Diffuse Large B-Cell Lymphoma in Patients Ineligible for or Having Failed Autologous Transplantation: A Single-Arm, Phase II Study. J Clin Oncol. 2019; 37(6):481-489. doi:10.1200/JCO.18.00766

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. A method of treating a hematologic malignancy in a subject in need thereof, said method comprising
a) detecting the expression of Programmed Death-Ligand 1 (PD-L1) in a biological sample of the subject; and
b) administering to the subject a therapeutically effective amount of an inhibitor of PD-L1 or Programmed Death 1 (PD-1), and a therapeutically effective amount of a T cell activation therapeutic, wherein PD-L1 expression is detected in the biological sample.

2. (canceled)

3. The method of claim 1, wherein in step b) PD-L1 expression is detected in at least 1%, at least 5%, or at least 10% of the cells in the biological sample.

4. The method of claim 3, wherein:
the cells are tumor cells, lymphocytes and/or macrophages; and/or
the cells are CD20+ cells.

5-6. (canceled)

7. The method of claim 1, wherein the inhibitor of PD-1 or PD-L1 is an antibody.

8. The method of claim 1, wherein the inhibitor of PD-1 or PD-L1 is pembrolizumab, nivolumab, pidilizumab, AMP-224, RMP1-4, J43, atezolizumab, avelumab, BMS-936559, durvalumab, tislelizumab, cemiplimab, or a combination thereof.

9. The method of claim 1, wherein:
a first dose of the inhibitor of PD-1 or PD-L1 is administered to the subject followed by one or more maintenance doses of the inhibitor of PD-1 or PD-L1; and/or
the inhibitor of PD-1 or PD-L1 is administered about every 1 to 9 weeks, or every 1 to 6 weeks, or every 2, 3, 4, or 6 weeks; and/or
the inhibitor of PD-1 or PD-L1 is administered before, after, or concurrently with the T cell activation therapeutic; and/or
the inhibitor of PD-1 or PD-L1 is administered at about 50 mg per dose to about 1500 mg per dose, or about 100 mg, 200 mg, 400 mg, 480 mg or 1200 mg per dose, less than 300 mg per dose, or about 200 mg/day.

10-15. (canceled)

16. The method of claim 1, wherein the T cell activation therapeutic comprises at least one survivin antigen.

17. (canceled)

18. The method of claim 16, wherein:
the survivin antigen is a peptide antigen comprising an amino acid sequence from the survivin protein (SEQ ID NO: 1) that is capable of eliciting a cytotoxic T-lymphocyte (CTL) response in the subject, or a nucleic acid molecule encoding said peptide antigen; or
the survivin antigen is a peptide antigen comprising at least one of amino acid sequence FEELTLGEF (SEQ ID NO: 2); FTELTLGEF (SEQ ID NO: 3); LTLGEFLKL (SEQ ID NO: 4); LMLGEFLKL (SEQ ID NO: 5); RISTFKNWPF (SEQ ID NO: 6); RISTFKNWPK (SEQ ID NO: 7); STFKNWPFL (SEQ ID NO: 8); or LPPAWQPFL (SEQ ID NO: 9), or a nucleic acid molecule encoding said peptide antigen; or
the at least one survivin antigen comprises a mixture of five peptide antigens comprising the amino acid sequence FTELTLGEF (SEQ ID NO: 3); LMLGEFLKL (SEQ ID NO: 5); RISTFKNWPK (SEQ ID NO: 7); STFKNWPFL (SEQ ID NO: 8) or LPPAWQPFL (SEQ ID NO: 9).

19-20. (canceled)

21. The method of claim 16, wherein the at least one survivin antigen is administered at a concentration of about 0.1 mg/ml to about 5 mg/ml, or about 1 mg/ml for each peptide antigen.

22. The method of claim 16, wherein the T cell activation therapeutic is a composition comprising the at least one survivin antigen, lipid vesicle particles, and a carrier comprising a continuous phase of a hydrophobic substance.

23. The method of claim 22, wherein the composition further comprises a T-helper epitope.

24. The method of claim 23, wherein the T-helper epitope is a peptide comprising the amino acid sequence AQYIKANSKFIGITEL (SEQ ID NO: 10).

25. The method of claim 22, wherein the composition further comprises an adjuvant.

26. (canceled)

27. The method of claim 25, wherein the adjuvant is a polyI.C polynucleotide and wherein the polyI.C polynucleotide is DNA or RNA based.

28. The method of claim 22, wherein the carrier is a vegetable oil, nut oil, mineral oil, or a mannide oleate in a mineral oil solution.

29-31. (canceled)

32. The method of claim 1, wherein step b) further comprises administering an effective amount of one or more active agent to the subject.

33. The method of claim 32, wherein the active agent is an agent that interferes with DNA replication.

34. (canceled)

35. The method of claim 33, wherein the active agent is cyclophosphamide.

36-37. (canceled)

38. The method of claim 32, wherein:
the active agent is administered at about 25-300 mg/day, about 50-100 mg/day, or about 100 mg/day; and/or
the active agent is administered at about 50 mg per dose; and/or
the active agent is administered to the subject at least 1, 2, 3, or 4 times daily; and/or
the active agent is administered before, after, or concurrently with the T cell activation therapeutic.

39-41. (canceled)

42. The method of claim 32, wherein:
step b) comprises administering a first dose of the active agent to the subject at least two days, at least four days, or about one week prior to administering the T cell activation therapeutic; and/or
step b) comprises administering to the subject a first dose of the active agent, followed by one or more maintenance doses of the active agent; and/or
step b) comprises administering the active agent to the subject twice daily for a period of about one week.

43-44. (canceled)

45. The method of claim 32, wherein step b) comprises administering the active agent to the subject in a low dose metronomic regimen, wherein:
the metronomic regimen comprises administering the active agent to the subject daily for a period of about one week every second week; or
the metronomic regimen comprises administering the active agent for a two-week cycle, wherein the active agent is administered to the subject during the first week of the cycle, wherein the active agent is not administered to the subject during the second week of the cycle, and wherein the metronomic regimen comprises at least two cycles.

46-47. (canceled)

48. The method of claim 32, wherein step b) comprises administering the active agent to the subject beginning about one week before administering a first dose of the T cell activation therapeutic, administering a second dose of the T cell activation therapeutic to the subject about three weeks after the first dose, and then administering the T cell activation therapeutic to the subject about once every eight weeks.

49. The method of claim 1, wherein the hematologic malignancy is non-Hodgkin lymphoma (NHL).

50. The method of claim 1, wherein the hematologic malignancy is diffuse large B cell lymphoma (DLBCL).

51. The method of claim 50, wherein the DLBCL is a relapsed/refractory DLBCL.

52. A method of identifying a subject who is likely to benefit from a combination treatment comprising an inhibitor of PD-L1 or Programmed Death 1 (PD-1) and a T cell activation therapeutic, wherein the subject has a hematologic malignancy, comprising
a) detecting the expression of Programmed Death-Ligand 1 (PD-L1) in a biological sample of the subject; and
b) identifying the subject as likely to benefit from the combination treatment comprising an inhibitor of PD-L1 or PD-1 and a T cell activation therapeutic, wherein PD-L1 expression is detected in the biological sample.

53-55. (canceled)
